(19) United States
(12) Patent Application Publication
(10) Pub. No.: US 2024/0025968 A1
(43) Pub. Date: Jan. 25, 2024

(54) LAG-3 TARGETED HETERODIMERIC FUSION PROTEINS CONTAINING IL-15/IL-15RA Fc-FUSION PROTEINS AND LAG-3 ANTIGEN BINDING DOMAINS

(71) Applicant: Xencor, Inc., Pasadena, CA (US)

(72) Inventors: Matthew J. Bernett, Monrovia, CA (US); John R. Desjarlais, Pasadena, CA (US); Suzanne Schubbert, Long Beach, CA (US)

(21) Appl. No.: 18/296,591

(22) Filed: Apr. 6, 2023

Related U.S. Application Data
(60) Provisional application No. 63/328,697, filed on Apr. 7, 2022.
(60) Provisional application No. 63/358,799, filed on Jul. 6, 2022.
(60) Provisional application No. 63/408,059, filed on Sep. 19, 2022.

(51) Int. Cl. C07K 14/715; C07K 14/54; C12N 15/63; C07K 16/28

(52) U.S. Cl. CPC C07K 14/7155; C07K 14/5443; C07K 2319/30; C07K 16/2896; C12N 15/63

(57) ABSTRACT

The present invention is directed to novel targeted heterodimeric fusion proteins comprising an IL-15/IL-15Rα Fc-fusion protein and a LAG-3 antibody fragment-Fc fusion protein.

### PRIORITY CLAIM

This application claims priority to and benefit of U.S. Provisional Application No. 63/328,697, filed on Apr. 7, 2022; 63/358,799, filed on Jul. 6, 2022; and 63/408,059, filed on Sep. 19, 2022, the contents of which are hereby incorporated by reference in their entireties.

### SEQUENCE LISTING

The instant application contains a Sequence Listing which has been submitted electronically in XML file format and is hereby incorporated by reference in its entirety. Said XML copy, created on Mar. 30, 2023, is named 067461-5300-WO_SL.xml and is 298,102 bytes in size.

### BACKGROUND OF THE INVENTION

Two very promising approaches in cancer immunotherapy include cytokine-based treatments and blockade of immune checkpoint proteins such as PD-1.

Cytokines such as IL-2 and IL-15 function in aiding the proliferation and differentiation of B cells, T cells, and NK cells. Both cytokines exert their cell signaling function through binding to a trimeric complex consisting of two shared receptors, the common gamma chain (γc; CD132) and IL-2 receptor beta-chain (IL-2Rβ; CD122), as well as an alpha chain receptor unique to each cytokine: IL-2 receptor alpha (IL-2Rα; CD25) or IL-15 receptor alpha (IL-15Rα; CD215). Both cytokines are considered as potentially valuable therapeutics in oncology, and IL-2 has been approved for use in patients with metastatic renal-cell carcinoma and malignant melanoma. Currently, there are no approved uses of recombinant IL-15, although several clinical trials are ongoing. However, as potential drugs, both cytokines suffer from a very fast clearance, with half-lives measured in minutes. IL-2 immunotherapy has been associated with systemic toxicity when administered in high doses to overcome fast clearance. Such systemic toxicity has also been reported with IL-15 immunotherapy in recent clinical trials (Guo et al., J Immunol, 2015, 195(5):2353-64).

Immune checkpoint proteins such as PD-1 are up-regulated following T cell activation to preclude autoimmunity by exhausting activated T cells upon binding to immune checkpoint ligands such as PD-L1. However, immune checkpoint proteins are also up-regulated in tumor-infiltrating lymphocytes (TILs), and immune checkpoint ligands are overexpressed on tumor cells, contributing to immune escape by tumor cells. De-repression of TILs by blockade of immune checkpoint interactions by drugs such as Opdivo® (nivolumab) and Keytruda® (pembrolizumab) have proven highly effective in treatment of cancer. Despite the promise of checkpoint blockade therapies such as nivolumab and pembrolizumab, many patients still fail to achieve sufficient response to checkpoint blockade alone.

Therefore, there remains an unmet need in oncology treatment for therapeutic strategies with cytokines that do not require high doses and are targeted to tumors to avoid systemic toxicity. Further, there is a need to identify additional therapeutic modalities to stack with checkpoint blockade that could increase patient response rate.

To address these needs and caveats, provided herein are novel LAG-3-targeted IL-15 heterodimeric fusion proteins with enhanced half-life and more selective targeting of TILs to improve safety profiles, and which synergistically combine with checkpoint blockade antibodies (FIG. 1).

### SUMMARY

The present invention provides targeted IL-15/IL-15Rα heterodimeric proteins comprising a human LAG-3 antigen binding domain.

In one aspect, provided are “scIL-15/Rα×Fab” format heterodimeric proteins. Such “scIL-15/Rα×Fab” format heterodimeric proteins include: a) a first monomer comprising, from N- to C-terminal: i) an IL-15Rα (sushi) domain; ii) a first domain linker; iii) an IL-15 variant; iv) a second domain linker; v) a first variant Fc domain comprising CH2-CH3; b) a second monomer comprising, from N- to C-terminal, VH-CH1-hinge-CH2-CH3, wherein CH2-CH3 is a second variant Fc domain; and c) a third monomer comprising a VL-CL. The VH and VL are a variable heavy domain and a variable light domain, respectively, that form a human LAG-3 antigen binding domain. In some embodiments, the second domain linker is an antibody hinge.

In certain embodiments of the “scIL-15/Rα×Fab” format heterodimeric protein, the first variant Fc domain the second variant Fc domain comprises one of the following skew variant sets: S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L and K370S: S364K/E357Q, according to EU numbering. In an exemplary embodiment, the skew variant set is S364K/E357Q: L368D/K370S.

In an exemplary embodiment of the “scIL-15/Rα×Fab” format heterodimeric protein, the “scIL-15/Rα×Fab” format heterodimeric protein includes: a) a first monomer comprising, from N- to C-terminal: i) an IL-15Rα (sushi) domain; ii) a first domain linker; iii) an IL-15 variant; iv) a hinge; v) a first variant Fc domain comprising CH2-CH3; b) a second monomer comprising, from N- to C-terminal, VH-CH1-hinge-CH2-CH3, wherein the CH2-CH3 is a second variant Fc domain; and c) a third monomer comprising a VL-CL. The VH and VL are a variable heavy domain and a variable light domain, respectively, that form a human LAG-3 antigen binding domain. In such embodiments, the first variant Fc domain comprises skew variants L368D/K370S and the second variant Fc domain comprises skew variants S364K/E357Q, the first and second variant Fc domains each comprise FcKO variants E233P/L234V/L235A/G236del/S267K, the first variant Fc domain comprises pI variants Q295E/N384D/Q418E/N421D, wherein numbering is according to EU numbering. In some embodiments, the hinge of the first monomer comprises amino acid substitution C220S, wherein numbering is according to EU numbering. In an exemplary embodiment, the first and second variant Fc domains each further comprise half-life extension variants M428L/N434S.

In some embodiments of the “scIL-15/Rα×Fab” format heterodimeric protein, the first domain linker is (GGGGA)₅ (SEQ ID NO: 25).

In some embodiments of the “scIL-15/Rα×Fab” format heterodimeric protein, the IL-15 variant of the heterodimeric protein provided herein comprises an amino acid substitution(s) selected from the group consisting of N1D, N4D, D8N, D30N, D61N, E64Q, N65D, N71Q, N79Q, Q108E, and N112Q. In an exemplary embodiment, the IL-15 variant comprises amino acid substitutions N4D/N65D, D30N/N65D, D30N/E64Q/N65D, D30N/E64Q/N65D/N71Q/N79Q/N, 112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N, 112Q, D30N/D61N/N65D/N71Q/N79Q/N112Q, or D8N/D30N/E64Q/N65D/N71Q/N79Q/N112Q.

In an exemplary embodiment, the IL-15 variant comprises amino acid substitutions D30N/E64Q/N65D/N71Q/N79Q/N, 112Q or N1D/N4D/D30N/E64Q/N65D/N71Q/N79Q/N, 112Q.

In an exemplary embodiment, the “scIL-15/Rα×Fab” format heterodimeric protein is XENP40735, XENP40741, XENP40736, or XENP40812.

In certain embodiment, the VH and VL of the “scIL-15/Rα×Fab” format heterodimeric proteins provided herein are the variable heavy domain and variable domain of any of the LAG-3 antigen binding domains in FIGS. 12 and 13A-13C. In an exemplary embodiment, the LAG-3 antigen binding domain is 2A11_H1.144_L2.142. In an exemplary embodiment, the LAG-3 antigen binding domain is 7G8_H3.30_L1.34.

In one aspect, provided herein are “mAb-scIL-15/Rα” format heterodimeric proteins. The “mAb-scIL-15/Rα” format heterodimeric proteins include: a) a first monomer comprising, from N- to C-terminal, VH-CH1-hinge-CH2-CH3, wherein the CH2-CH3 is a first variant Fc domain; b) a second monomer comprising, from N- to C-terminal, VH-CH1-hinge-CH2-CH3-first domain linker-IL-15Rα (sushi) domain-second domain linker-IL-15 variant, wherein the CH2-CH3 is a second variant Fc domain; and c) a third monomer and fourth monomer that each comprises, from N- to C-terminal, VL-CL. Further, the VH of the first monomer and the VL of the third monomer form a first human LAG-3 binding domain, and the VH of the second monomer and the VL of the fourth monomer form a second human LAG-3 binding domain.

In some embodiments of the “mAb-scIL-15/Rα” format heterodimeric protein, the first variant Fc domain the second variant Fc domain comprises one of the following skew variant sets: S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L and K370S: S364K/E357Q, according to EU numbering. In an exemplary embodiment, the skew variant set is S364K/E357Q: L368D/K370S.

In some embodiments of the “mAb-scIL-15/Rα” format heterodimeric protein, the first variant Fc domain comprises skew variants L368D/K370S and the second variant Fc domain comprises skew variants S364K/E357Q, and the first and second variant Fc domains each comprise FcKO variants E233P/L234V/L235A/G236del/S267K, wherein numbering is according to EU numbering. In certain embodiments, a) the hinge-first variant Fc domain of the first monomer further comprises pI substitutions N208D/Q295E/N384D/Q418D/N421D and the hinge-second variant Fc domain of the second monomer further comprises pI variants Q196K/I199T/P271R/P228R/N276K; b) the hinge-first variant Fc domain of the first monomer further comprises pI substitutions N208D/Q295E/N384D/Q418D/N421D; or c) the hinge-second variant Fc domain of the second monomer further comprises pI variants Q196K/I199T/P271R/P228R/N276K, wherein numbering is according to EU numbering. In such embodiments, the first variant Fc domain comprises skew variants L368D/K370S and the second variant Fc domain comprises skew variants S364K/E357Q, the first and second variant Fc domains each comprise FcKO variants E233P/L234V/L235A/G236del/S267K, the first variant Fc domain comprises pI variants Q295E/N384D/Q418E/N421D, wherein numbering is according to EU numbering.

In some embodiments of the “mAb-scIL-15/Rα” format heterodimeric protein, the first variant Fc domain comprises skew variants S364K/E357Q and the second variant Fc domain comprises skew variants L368D/K370S, and the first and second variant Fc domains each comprise FcKO variants E233P/L234V/L235A/G236del/S267K, wherein numbering is according to EU numbering. In such embodiments, a) the hinge-first variant Fc domain of the first monomer further comprises pI substitutions Q196K/I199T/P271R/P228R/N276K and the hinge-second variant Fc domain of the second monomer further comprises pI variants N208D/Q295E/N384D/Q418D/N421D; b) the hinge-first variant Fc domain of the first monomer further comprises pI substitutions Q196K/I199T/P271R/P228R/N276K; or c) the hinge-second variant Fc domain of the second monomer further comprises pI variants N208D/Q295E/N384D/Q418D/N421D, wherein numbering is according to EU numbering.

In some embodiments of the “mAb-scIL-15/Rα” format heterodimeric protein, the first and second variant Fc domains each further comprise half-life extension variants M428L/N434S.

In some embodiments of the “mAb-scIL-15/Rα” format heterodimeric protein, the IL-15 variant of the heterodimeric protein provided herein comprises an amino acid substitution(s) selected from the group consisting of N1D, N4D, D8N, D30N, D61N, E64Q, N65D, N71Q, N79Q, Q108E, and N112Q. In an exemplary embodiment, the IL-15 variant comprises amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q.

In an exemplary embodiment, the IL-15 variant comprises amino acid substitutions N4D/D30N/E64Q/N65D.

In an exemplary embodiment, the “mAb-scIL-15/Rα” format heterodimeric protein is XENP40814 or XENP40738.

In one aspect, provided herein are “hetero-mAb-IL-15/Rα” format heterodimeric proteins. The “hetero-mAb-IL-15/Rα” format heterodimeric proteins include: a) a first monomer comprising, from N- to C-terminal, VH-CH1-hinge-CH2-CH3-first domain linker-IL-15Rα (sushi) domain, wherein the CH2-CH3 is a first variant Fc domain; b) a second monomer comprising, from N- to C-terminal, VH-CH1-hinge-CH2-CH3-second domain linker-IL-15 variant, wherein the CH2-CH3 is a second variant Fc domain; and c) a third monomer and fourth monomer that each comprises, from N- to C-terminal, VL-CL. Further, the VH of the first monomer and the VL of the third monomer form a first human LAG-3 binding domain, and the VH of the second monomer and the VL of the fourth monomer form a second human LAG-3 binding domain.

In some embodiments of the “hetero-mAb-IL-15/Rα” format heterodimeric protein, the first variant Fc domain the second variant Fc domain comprises one of the following skew variant sets: S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L and K370S: S364K/E357Q, according to EU numbering. In an exemplary embodiment, the skew variant set is S364K/E357Q: L368D/K370S.

In some embodiments of the “hetero-mAb-IL-15/Rα” format heterodimeric protein, the first variant Fc domain comprises skew variants L368D/K370S and the second variant Fc domain comprises skew variants S364K/E357Q, and the first and second variant Fc domains each comprise FcKO variants E233P/L234V/L235A/G236del/S267K, wherein numbering is according to EU numbering. In certain embodiments, a) the hinge-first variant Fc domain of the first monomer further comprises pI substitutions N208D/Q295E/N384D/Q418D/N421D and the hinge-second variant Fc domain of the second monomer further comprises pI variants Q196K/I199T/P271R/P228R/N276K; b) the hinge-first variant Fc domain of the first monomer further comprises pI substitutions N208D/Q295E/N384D/Q418D/N421D; or c) the hinge-second variant Fc domain of the second monomer further comprises pI variants Q196K/I199T/P271R/P228R/N276K, wherein numbering is according to EU numbering. In such embodiments, the first variant Fc domain comprises skew variants L368D/K370S and the second variant Fc domain comprises skew variants S364K/E357Q, the first and second variant Fc domains each comprise FcKO variants E233P/L234V/L235A/G236del/S267K, the first variant Fc domain comprises pI variants Q295E/N384D/Q418E/N421D, wherein numbering is according to EU numbering.

In some embodiments of the “hetero-mAb-IL-15/Rα” format heterodimeric protein, the first variant Fc domain comprises skew variants S364K/E357Q and the second variant Fc domain comprises skew variants L368D/K370S, and the first and second variant Fc domains each comprise FcKO variants E233P/L234V/L235A/G236del/S267K, wherein numbering is according to EU numbering. In such embodiments, a) the hinge-first variant Fc domain of the first monomer further comprises pI substitutions Q196K/I199T/P271R/P228R/N276K and the hinge-second variant Fc domain of the second monomer further comprises pI variants N208D/Q295E/N384D/Q418D/N421D; b) the hinge-first variant Fc domain of the first monomer further comprises pI substitutions Q196K/I199T/P271R/P228R/N276K; or c) the hinge-second variant Fc domain of the second monomer further comprises pI variants N208D/Q295E/N384D/Q418D/N421D, wherein numbering is according to EU numbering.

In some embodiments of the “hetero-mAb-IL-15/Rα” format heterodimeric protein, the first and second variant Fc domains each further comprise half-life extension variants M428L/N434S.

In some embodiments of the “hetero-mAb-IL-15/Rα” format heterodimeric protein, the IL-15 variant of the heterodimeric protein provided herein comprises an amino acid substitution(s) selected from the group consisting of N1D, N4D, D8N, D30N, D61N, E64Q, N65D, N71Q, N79Q, Q108E, and N112Q. In an exemplary embodiment, the IL-15 variant comprises amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the IL-15 variant comprises amino acid substitutions N4D/D30N/E64Q/N65D.

In an exemplary embodiment, the “hetero-mAb-IL-15/Rα” format heterodimeric protein is XENP40816.

In certain embodiment, the VH and VL of any of the heterodimeric proteins provided herein are the variable heavy domain and variable domain of any of the LAG-3 antigen binding domains in FIGS. 12 and 13A-13C. In an exemplary embodiment, the LAG-3 antigen binding domain is 2A11_H1.144_L2.142. In an exemplary embodiment, the LAG-3 antigen binding domain is 7G8_H3.30_L1.34.

In some embodiments, the IL-15 variant of the heterodimeric protein provided herein comprises an amino acid substitution(s) selected from the group consisting of N1D, N4D, D8N, D30N, D61N, E64Q, N65D, Q108E, N4D/N65D, D30N/N65D, and D30N/E64Q/N65D. In an exemplary embodiment, the IL-15 variant comprises amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q

In one aspect, provided herein is a pharmaceutical composition that includes any of the heterodimeric proteins disclosed herein and a pharmaceutically acceptable carrier.

In another aspect, provided herein is a method of treating a patient in need thereof comprising administering to the patient any one of the heterodimeric proteins or pharmaceutical compositions disclosed herein. In some embodiments, the method further comprising administering an antibody, where the antibody is an anti-PD-1 antibody, an anti-PD-L1 antibody, an anti-CTLA-4 antibody, an anti-TIM-3 antibody or an-anti-TIGIT antibody.

In another aspect, provided herein are nucleic acid compositions that include one or more nucleic acids encoding any of the heterodimeric proteins disclosed herein, expression vectors that include the nucleic acids, host cells that include the nucleic acids or expression vectors. Also provided herein are methods of making subject heterodimeric proteins by culturing host cells under suitable conditions and recovering the heterodimeric proteins.

### BRIEF DESCRIPTION OF THE DRAWINGS

FIG. 1 selectivity of LAG-3-targeted IL-15/Rα-Fc fusion proteins for tumor-reactive tumor-infiltrating lymphocytes expressing PD-1, and its combination with PD-1 blockade antibody.

FIGS. 2A-2B depict the sequences for IL-15 and its receptors.

FIG. 3 depicts the sequences for LAG-3, including both human and cyno (predicted), to facilitate the development of antigen binding domains that bind to both for ease of clinical development.

FIGS. 4A-4E depict useful pairs of Fc heterodimerization variant sets (including skew and pI variants). There are variants for which there are no corresponding “monomer 2” variants; these are pI variants which can be used alone on either monomer.

FIG. 5 depicts a list of isosteric variant antibody constant regions and their respective substitutions. pI_(−) indicates lower pI variants, while pI_(+) indicates higher pI variants. These can be optionally and independently combined with other heterodimerization variants of the invention (and other variant types as well, as outlined herein).

FIG. 6 depicts useful ablation variants that ablate FcγR binding (sometimes referred to as “knock outs” or “KO” variants). Generally, ablation variants are found on both monomers, although in some cases they may be on only one monomer.

FIGS. 7A-7F show particularly useful embodiments of “non-cytokine”/“non-Fv” components of the LAG-3-targeting IL-15/Rα-Fc fusion proteins of the invention.

FIG. 8 depicts a number of exemplary variable length linkers for use in IL-15/Rα-Fc fusion proteins. In some embodiments, these linkers find use linking the C-terminus of IL-15 and/or IL-15Rα (sushi) to the N-terminus of the Fc region. In some embodiments, these linkers find use fusing IL-15 to the IL-15Rα (sushi). While the “hinge” based domain linkers in this Figure are based on IgG1 hinge, hinge regions from other IgG isotypes may also be used. In the case of IgG2, the hinge sequence may include C219S and/or C220S substitutions. Additionally, each of these domain linkers may be used in multiples (e.g. EAAAKEAAAK (SEQ ID NO: 61)) or in combination (e.g. EAAAKEPKSSDKTHTCPPCP (SEQ ID NO: 62)).

FIGS. 9A-9C depict a number of charged scFv linkers that find use in increasing or decreasing the pI of heterodimeric antibodies that utilize one or more scFv as a component. The (+H) positive linker finds particular use herein. A single prior art scFv linker with single charge is referenced as “Whitlow”, from Whitlow et al., Protein Engineering 6(8):989-995(1993). It should be noted that this linker was used for reducing aggregation and enhancing proteolytic stability in scFvs.

FIG. 10 shows the sequences of several useful LAG-3-targeting IL-15/Rα-Fc fusion format backbones based on human IgG1, without the cytokine sequences (e.g., the Il-15 and/or IL-15Rα (sushi)) or VH, and further excluding light chain backbones which are depicted in FIG. 11. Backbone 1 is based on human IgG1 (356E/358M allotype), and includes the S364K/E357Q: L368D/K370S skew variants, C220S and the Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes the S364K/E357Q: L368D/K370S skew variants, the N208D/Q295E/N384D/Q418E/N421D pI variants on the chain with L368D/K370S skew variants, C220S in the chain with S364K/E357Q variants, and the E233P/L234V/L235A/G236del/S267K ablation variants on both chains. Backbone 3 is based on human IgG1 (356E/358M allotype), and includes the S364K/E357Q: L368D/K370S skew variants, the N208D/Q295E/N384D/Q418E/N421D pI variants on the chains with L368D/K370S skew variants, the Q196K/I199T/P217R/P228R/N276K pI variants on the chains with S364K/E357Q variants, and the E233P/L234V/L235A/G236del/S267K ablation variants on both chains. Such backbone sequences can be included, for example, in the “scIL-15/Rα×Fab” format heterodimeric proteins described herein. In some embodiments, the “scIL-15/Rα×Fab” format heterodimeric protein includes: a) a first monomer that includes, from N- to C-terminus, an IL-15Rα (sushi) domain-(domain linker)-IL-15 variant-(hinge)-CH2-CH3, where hinge-CH2-CH3 has the amino acid sequence of “Chain 2” of any of the backbone sequences in FIG. 10 (SEQ ID NO: 68, 70, and 72); b) a second monomer that includes, from N- to C-terminus, a VH-CH1-hinge-CH2-CH3, where VH is a variable heavy domain and CH1-hinge-CH2-CH3 has the amino acid sequence of Chain 1 of any one of the backbone sequences in FIG. 10 (SEQ ID NO: 67, 69, and 71), and c) a light chain that includes from, N- to C-terminus, VL-VC, where VL is a variable light domain and VC has the sequence of “Constant Light Chain-Kappa” or “Constant Light Chain-Lambda” in FIG. 11 (SEQ ID NO: 73-74). In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In exemplary embodiments, the VH and VL are the variable heavy domain and variable light domain, respectively, of any of the LAG-3 ABDs provided in FIGS. 12 and 13A-C.

In certain embodiments, these sequences can be of the 356D/358L allotype. In other embodiments, these sequences can include either the N297A or N297S substitutions. In some other embodiments, these sequences can include the M428L/N434S Xtend mutations. In yet other embodiments, these sequences can instead be based on human IgG4, and include a S228P (EU numbering, this is S241P in Kabat) variant on both chains that ablates Fab arm exchange as is known in the art. In yet further embodiments, these sequences can instead be based on human IgG2. Further, these sequences may instead utilize the other skew variants, pI variants, and ablation variants depicted in the Figures.

As will be appreciated by those in the art and outlined below, these sequences can be used with any IL-15 and IL-15Rα (sushi) pairs outlined herein, including but not limited to scIL-15/Rα, ncIL-15/Rα, and dsIL-15Rα, as schematically depicted in FIGS. 17A-17L. Further as will be appreciated by those in the art and outlined below, any IL-15 and/or IL-15Rα (sushi) variants can be incorporated in these backbones. Furthermore as will be appreciated by those in the art and outlined below, these sequences can be used with any VH and VL pairs outlined herein, including either a scFv or a Fab.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 11 depicts the “non-Fv” backbone of light chains (i.e. constant light chain) which find use in LAG-3-targeting IL-15/Rα-Fc fusion proteins of the invention.

FIG. 12 depicts the variable region sequences for a select number of anti-LAG-3 antibody binding domains. The CDRs are underlined. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 13A-13C depict the variable regions of additional LAG-3 ABDs which may find use in the LAG-3-targeting IL-15/Rα-Fc fusion proteins of the invention. The CDRs are underlined. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 14 depicts the variable heavy domain, variable light domain, and CDRs of the anti-LAG3 binding domain 7G8[LAG3]_H3.30_L1.34.

FIG. 15 depicts the structure of IL-15 complexed with IL-15Rα, IL-2Rβ, and common gamma chain. Locations of substitutions designed to reduce potency are shown.

FIGS. 16A-16C depict sequences for illustrative IL-15 variants engineered for reduced potency and also, in some instances, better pharmacokinetics in vivo. Included within each of these variant IL-15 sequences are sequences that are 90, 95, 98 and 99% identical (as defined herein) to the recited sequences, and/or contain from 1, 2, 3, 4, 5, 6, 7, 8, 9 or 10 additional amino acid substitutions. In a non-limiting example, the recited sequences may contain additional amino acid modifications such as those contributing to formation of covalent disulfide bonds.

FIGS. 17A-17L depicts various formats of LAG-3 targeted IL-15. FIG. 17A, FIG. 17B, FIG. 17C, FIG. 17D, FIG. 17E, FIG. 17F, FIG. 17G, FIG. 17H, FIG. 17I, FIG. 17J, FIG. 17K and FIG. 17L depict several formats for the LAG-3-targeting IL-15/Rα-Fc fusion proteins of the present invention. The “scIL-15/Rα×scFv” format (FIG. 17A) comprises IL-15Rα (sushi) fused to IL-15 by a variable length linker (termed “scIL-15/Rα”) which is then fused to the N-terminus of a heterodimeric Fc-region, with an scFv fused to the other side of the heterodimeric Fc. The “scFv×ncIL-15/Rα” format (FIG. 17B) comprises an scFv fused to the N-terminus of a heterodimeric Fc-region, with IL-15Rα (sushi) fused to the other side of the heterodimeric Fc, while IL-15 is transfected separately so that a non-covalent IL-15/Rα complex is formed. The “scFv×dsIL-15/Rα” format (FIG. 17C) is the same as the “scFv×ncIL-15/Rα” format, but wherein IL-15Rα (sushi) and IL-15 are covalently linked as a result of engineered cysteines. The “scIL-15/Rα×Fab” format (FIG. 17D) comprises IL-15Rα (sushi) fused to IL-15 by a variable length linker (termed “scIL-15/Rα”) which is then fused to the N-terminus of a heterodimeric Fc-region, with a variable heavy chain (VH) fused to the other side of the heterodimeric Fc, while a corresponding light chain is transfected separately so as to form a Fab with the VH. The “ncIL-15/Rα×Fab” format (FIG. 17E) comprises a VH fused to the N-terminus of a heterodimeric Fc region, with IL-15Rα (sushi) fused to the other side of the heterodimeric Fc, while a corresponding light chain is transfected separately so as to form a Fab with the VH, and while IL-15 is transfected separately so that a non-covalent IL-15/Rα complex is formed. The “dsIL-15/Rα×Fab” format (FIG. 17F) is the same as the “ncIL-15/Rα×Fab” format, but wherein IL-15Rα (sushi) and IL-15 are covalently linked as a result of engineered cysteines. The “mAb-scIL-15/Rα” format (FIG. 17G) comprises VH fused to the N-terminus of a first and a second heterodimeric Fc, with IL-15 is fused to IL-15Rα (sushi) which is then further fused to the C-terminus of one of the heterodimeric Fc-region, while corresponding light chains are transfected separately so as to form a Fabs with the VHs. The “mAb-ncIL-15/Rα” format (FIG. 17H) comprises VH fused to the N-terminus of a first and a second heterodimeric Fc, with IL-15Rα (sushi) fused to the C-terminus of one of the heterodimeric Fc-region, while corresponding light chains are transfected separately so as to form a Fabs with the VHs, and while and while IL-15 is transfected separately so that a non-covalent IL-15/Rα complex is formed. The “mAb-dsIL-15/Rα” format (FIG. 17I) is the same as the “mAb-ncIL-15/Rα” format, but wherein IL-15Rα (sushi) and IL-15 are covalently linked as a result of engineered cysteines. The “central-IL-15/Rα” format (FIG. 17J) comprises a VH recombinantly fused to the N-terminus of IL-15 which is then further fused to one side of a heterodimeric Fc and a VH recombinantly fused to the N-terminus of IL-15Rα (sushi) which is then further fused to the other side of the heterodimeric Fc, while corresponding light chains are transfected separately so as to form a Fabs with the VHs. The “central-scIL-15/Rα” format (FIG. 17K) comprises a VH fused to the N-terminus of IL-15Rα (sushi) which is fused to IL-15 which is then further fused to one side of a heterodimeric Fc and a VH fused to the other side of the heterodimeric Fc, while corresponding light chains are transfected separately so as to form a Fabs with the VHs. The “hetero-mAb-IL-15/Rα” (FIG. 17L) format comprises VH fused to the N-terminus of a first and a second heterodimeric Fc, with IL-15 is fused to the C-terminus of one heterodimeric Fc-region, while IL-15Rα (sushi) is fused to the C-terminus of the other heterodimeric Fc-region, and corresponding light chains are transfected separately so as to form a Fabs with the VHs.

FIGS. 18A-18C depict the sequences for exemplary LAG-3×IL-15 constructs of the invention having the scIL15/Rα-Fab format, as depicted in FIG. 17D, including XENP40735, XENP40736, XENP40741, and XENP40812.

FIGS. 19A-19B depict the sequences of exemplary LAG3×IL15 constructs in the mAb-scIL-15/Rα format (as illustrated in FIG. 17G), including XENP40378 and XENP40814.

FIG. 20 depicts the sequences of an exemplary LAG3×IL15 construct in the bivalent mAb-Fv format, XENP40816.

FIGS. 21A-21I, depict the sequences of RSV×IL-15 test articles used as experimental controls.

FIGS. 22A-22E depict an experiment in which human PBMCs were activated with 500 ng/ml plate-bound anti-CD3 (OKT3) for 48 hours. Cells were labeled with carboxyfluorescein succinimidyl ester (CFSE). Next, either LAG-3×IL-15 constructs or RSV×IL-15 constructs were added to the cells at the range of concentrations indicated in the figure. After 4 days at 37° C., the cells were analyzed by flow cytometry in order to measure their proliferation by the concentration of CFSE. FIG. 22A, FIG. 22B, and FIG. 22C depict the percentage of proliferating cells and cell counts of CD8 effector memory T cells. FIGS. 22D-22E depict the percentage of proliferating cells and cell counts of NK cells (all CD56 and CD16+). The results demonstrated that LAG-3×IL-15 constructs have high selectivity for LAG3+ cell phenotypes such as CD8-effector memory T cells, and very low activity on cells with low LAG-3 expression, such as NK cells. For brevity, in the figure legends throughout, “1+1” refers to the scIL-15/Rα×Fab format.

FIGS. 23A-23C depict the results of a GVHD mouse model study in which NSG mice were engrafted with huPBMCs and then treated with LAG-3×IL-15 test articles or RSV×IL-15 test articles. FIG. 23A depicts the CD8+ T cell counts per 45 μl of blood. FIG. 23B depicts the body weights of mice on Day 13 of the study. FIG. 23C depicts the IFNγ concentration at Day 7 of the study.

FIG. 24A-24B depict drug concentrations (PK) and lymphocyte counts (PD) over time in a study in which a single dose of LAG-3×IL-15 was administered to non human primates (cynomolgus monkeys). The results demonstrate that both XENP40735 and XENP40736 have a superior PK as compared to wild-type IL-15-Fc. FIG. 24B depicts that LAG-3×IL-15 constructs are selective for CD8 effector memory T cells that are LAG-3+ compared to CD8 effector memory T cells in general.

FIGS. 25A-25E depict additional exemplary LAG-3×IL-15 sequences of the invention.

FIG. 26 depicts the ability of XENP40736 to induce strong anti-tumor activity in vivo. In this study, female CD34+ huNSG mice were each inoculated with 3×10⁶ pp65-MCF7-gfp cells on Day −15. On Day 0, one group of mice was treated with 3 mg/kg XENP16432, while a 2nd group was treated with 1 mg/kg XENP40736, and a 3rd group was left untreated (dosed with PBS only). Treatments were delivered in 0.1 ml volume intraperitonially, and were repeated once a week for 5 weeks. Tumor volume was measured by caliper 1-3× per week, and body weight measurements and blood draws were performed weekly.

FIG. 27 depicts the sequence of XENP16432, an anti-PD-1 based on nivolumab with IgG1-PVA_/S267K FcKO variant.

FIGS. 28A-28C depict XENP40735 and XENP40736 promoting anti-tumor activity, T-cell expansion, and IFNγ release in vivo. In this study, female CD34+ huNSG mice were each inoculated with 3×106 pp65-MCF7-gfp cells on Day −15. On Day 0, mice were treated with a range of doses of either XENP40735 or XENP40736, each either as a single agent or in combination with anti-PD1 XENP16432, as indicated in the legend of FIG. 28A. Treatments were delivered in 0.1 ml volume intraperitonially, and were repeated once a week for 5 weeks. Tumor volume was measured by caliper 1-3× per week, and body weight measurements and blood draws were performed weekly. Peripheral blood samples were stained with an antibody cocktail against surface and intracellular antigens and analyzed by flow cytometry to enumerate cell subsets. Cell count values of 0 were set to 1 for analyses and plotting. FIG. 28B depicts CD8 effector memory T cell counts at Day 14, and FIG. 28C depicts serum interferon gamma concentrations at Day 7.

FIG. 29 depicts the sequence of an anti-LAG3 bivalent antibody, XENP30473.

### DETAILED DESCRIPTION

### I. Definitions

In order that the application may be more completely understood, several definitions are set forth below. Such definitions are meant to encompass grammatical equivalents.

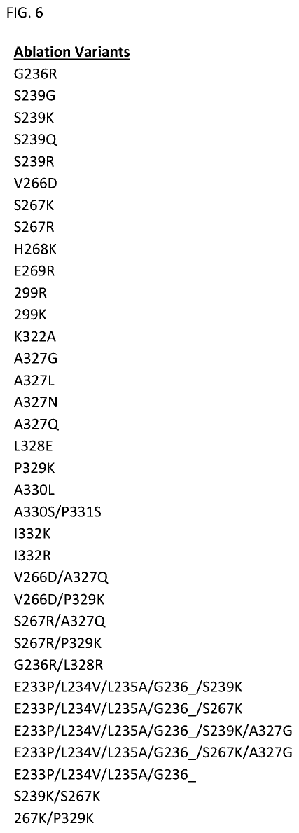

By “ablation” herein is meant a decrease or removal of activity. Thus for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. Of particular use in the ablation of FcγR binding are those shown in FIG. 6. However, unless otherwise noted, the Fc monomers of the invention retain binding to the FcRn receptor.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the invention ablate ADCC activity entirely.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

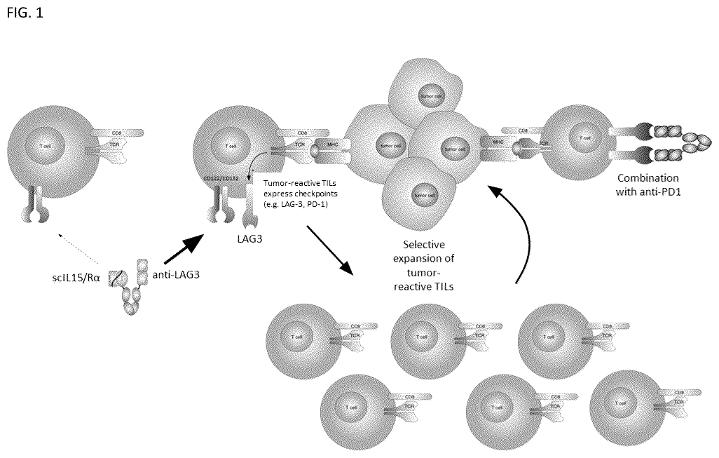

By “antigen binding domain” or “ABD” herein is meant a set of six Complementary Determining Regions (CDRs) that, when present as part of a polypeptide sequence, specifically binds a target antigen as discussed herein. Thus, a “LAG-3 antigen binding domain” binds a human LAG-3 antigen as outlined herein. As is known in the art, these CDRs are generally present as a first set of variable heavy CDRs (vhCDRs or V_{H}CDRs) and a second set of variable light CDRs (vlCDRs or V_{L}CDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (VH or vh or V_{H}; containing the vhCDR1, vhCDR2 and vhCDR3) and the variable light domain (VL or vl or V_{L}; containing the vlCDR1, vlCDR2 and vlCDR3), with the C-terminus of the VH domain being attached to the N-terminus of the CH1 domain of the heavy chain and the C-terminus of the vl domain being attached to the N-terminus of the constant light domain (and thus forming the light chain). In a scFv format, the VH and VL domains are covalently attached, generally through the use of a linker as outlined herein, into a single polypeptide sequence, which can be either (starting from the N-terminus) VH-linker-VL or VL-linker-VH, with the former being generally preferred (including optional domain linkers on each side, depending on the format used (e.g., from FIG. 1 of U.S. 62/353,511).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 # designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it.

“Variant,” as used herein can also refer to particular amino acid modifications (e.g., substitutions, deletions, insertions) in a variant protein (e.g., a variant Fc domain), for example, heterodimerization variants, ablation variants, FcKO variants, etc., as disclosed in Section II.C. below.

Accordingly, by “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference). The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full-length antibody, antibody fragment or Fab fusion protein.

By “Fv” or “Fv fragment” or “Fv region” as used herein is meant a polypeptide that comprises the VL and VH domains of a single antibody. As will be appreciated by those in the art, these generally are made up of two chains, or can be combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH).

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

By “FcRn” or “neonatal Fc Receptor” as used herein is meant a protein that binds the IgG antibody Fc region and is encoded at least in part by an FcRn gene. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers of the invention retain binding to the FcRn receptor (and, as noted below, can include amino acid variants to increase binding to the FcRn receptor).

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

By “Fc” or “Fc region” or “Fc domain” as used herein is meant the polypeptide comprising the constant region of an antibody excluding the first constant region immunoglobulin domain (e.g., CH1) and in some cases, part of the hinge. For IgG, the Fc domain comprises immunoglobulin domains CH2 and CH3 (C72 and C73) and the lower hinge region between CH1 (C71) and CH2 (C72). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in certain embodiments, wherein the Fc domain is a human IgG1 Fc domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3).

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

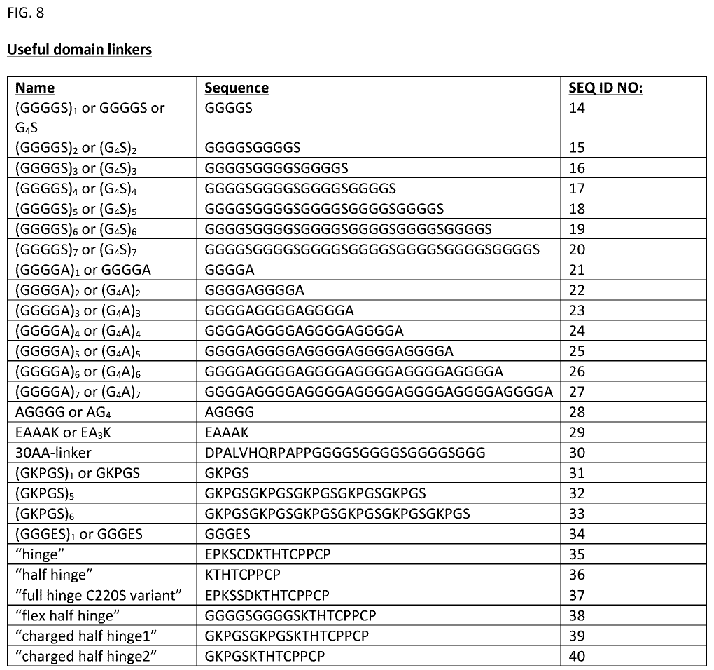

In the embodiments herein, when a scFv or IL-15 complex is attached to an Fc domain, it is the C-terminus of the scFv, IL-15 or IL-15Rα construct that is attached to the Fc domain via a domain linker; for example, a hinge domain as depicted in FIG. 8. In some embodiments, as is more fully described below, amino acid modifications are made to the Fc region, for example to alter binding to one or more FcγR receptors or to the FcRn receptor, and to enable heterodimer formation and purification, as outlined herein.

By “heavy constant region” herein is meant the CH1-hinge-CH2-CH3 portion of an antibody.

By “Fc fusion protein” or “immunoadhesin” herein is meant a protein comprising an Fc region, generally linked (optionally through a linker moiety, as described herein) to a different protein, such as to IL-15 and/or IL-15R, as described herein. In some instances, two Fc fusion proteins can form a homodimeric Fc fusion protein or a heterodimeric fusion protein with the latter being preferred. In some cases, one monomer of the heterodimeric fusion protein comprises an Fc domain alone (e.g., an empty Fc domain) and the other monomer is a Fc fusion, comprising a variant Fc domain and a protein domain, such as a receptor, ligand or other binding partner.

By “position” as used herein is meant a location in the sequence of a protein. Positions may be numbered sequentially, or according to an established format, for example the EU index for antibody numbering.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses the target antigen, in this case, LAG-3.

By “variable region” as used herein is meant the region of an immunoglobulin that comprises one or more Ig domains substantially encoded by any of the Vx, VX, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The subject LAG-3 targeted heterodimeric proteins are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins having different binding specificities. “Recombinant” means the proteins are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Specific binding” or “specifically binds to” or is “specific for” a particular antigen or an epitope (in this case, human LAG-3) means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay.

### II. Introduction

The invention provides heterodimeric fusion proteins that contain an IL-15 complex on one side and an anti-human LAG-3 antigen binding domain on the other. Thus, the heterodimeric fusion proteins of the invention can bind to the checkpoint LAG-3 antigen and can complex with the common gamma chain (γc; CD132) and/or the IL-2 receptor β-chain (IL-2Rβ; CD122). In general, the heterodimeric fusion proteins of the invention have three functional components: an IL-15/IL-15Rα (sushi) component, generally referred to herein as an “IL-15 complex”, an anti-LAG-3 ABD component which serves as a “targeting” moiety by bringing the fusion protein to a cell expressing LAG-3, and an Fc component, each of which can take different forms and each of which can be combined with the other components in any configuration.

In general, as is more fully described herein, the fusion proteins of the invention are heterodimeric proteins that are based on the association of antibody Fc domains. That is, by using two different variant Fc domains that have been engineered to favor the formation of heterodimers over homodimers, the heterodimeric proteins are formed. In this case, one of the variant Fc domains is fused to an IL-15/RA complex and the other has a LAG-3 ABD as more fully outlined herein. By including optional pI variants, the heterodimers can be more easily purified away from the homodimers. Additionally, the inclusion of ablation variants eliminates the effector functions of the Fc domains.

A. IL-15/IL-15Rα(Sushi) Domains

As shown in the figures, the IL-15 complex can take several forms. As stated above, the IL-15 protein on its own is less stable than when complexed with the IL-15Rα protein. As is known in the art, the IL-15Rα protein contains a “sushi domain”, which is the shortest region of the receptor that retains IL-15 binding activity. Thus, while heterodimeric fusion proteins comprising the entire IL-15Rα protein can be made, preferred embodiments herein include complexes that just use the sushi domain, the sequence of which is shown in the figures.

Accordingly, the IL-15 complex generally comprises the IL-15 protein and the sushi domain of IL IL-15Rα (unless otherwise noted that the full length sequence is used, “IL-15Rα”, “IL-15Rα (sushi)”, “IL-15RA” and “sushi” are used interchangeably throughout).

Importantly, the IL-15 component is generally engineered to reduce its potency. In many embodiments, the wild-type IL-15 is too potent and can cause undesirable toxicity. Accordingly, the IL-15 component of the IL-15 complex can have one or more amino acid substitutions that result in decreased activity. Various amino acid substitutions were made (see FIGS. 16A-16C). Of particular interest in some embodiments are a double variant, N4D/N65D or D30N/N65D, or a triple variant, D30N/E64Q/N65D. Of further interest in some embodiments are a quadruple variant, N4D/D30N/E64Q/N65D, a quintuple variant, N1D/N4D/D30N/E64Q/N65D, a sextuple variant, D30N/D61N/N65D/N71Q/N79Q/N112Q or D30N/E64Q/N65D/N71Q/N79Q/N112Q, a septuple variant, D30N/D61N/E64Q/N65D/N71Q/N79Q/N112Q or D8N/D30N/E64Q/N65D/N71Q/N79Q/N112Q112Q, or an octuple variant, N1D/N4D/D30N/E64Q/N65D/N71Q/N79Q/N112Q.

The targeted IL-15/IL-15Rα heterodimeric fusion proteins of the present invention include in some embodiments an IL-15/IL-15 receptor alpha (IL-15Rα)-Fc fusion monomer; reference is made to US2018/0118828, filed Oct. 16, 2017, U.S. Ser. No. 62/408,655, filed on Oct. 14, 2016, U.S. Ser. No. 62/416,087, filed on Nov. 1, 2016, U.S. Ser. No. 62/443,465, filed on Jan. 6, 2017, U.S. Ser. No. 62/477,926, filed on Mar. 28, 2017, and U.S. Ser. No. 62/659,571, filed on Apr. 18, 2018, hereby incorporated by reference in their entirety and in particular for the sequences outlined therein. In some cases, the IL-15 and IL-15 receptor alpha (IL-15Rα) protein domains are in different orientations. Exemplary embodiments of IL-15/IL-15Rα-Fc fusion monomers are provided in XENP21480 (chain 1; FIG. 64A), XENP22022 (chain 1, FIG. 64D), XENP22112, (chains 1 and 3; FIG. 64E), XENP22641 (chains 2 and 4; FIG. 64F), XENP22642, (chains 1 and 4; FIG. 64H) and XENP22644 (chains 1 and 4; FIG. 64I) as described, for example, in US 2018/0118828.

1. IL-15 Variants

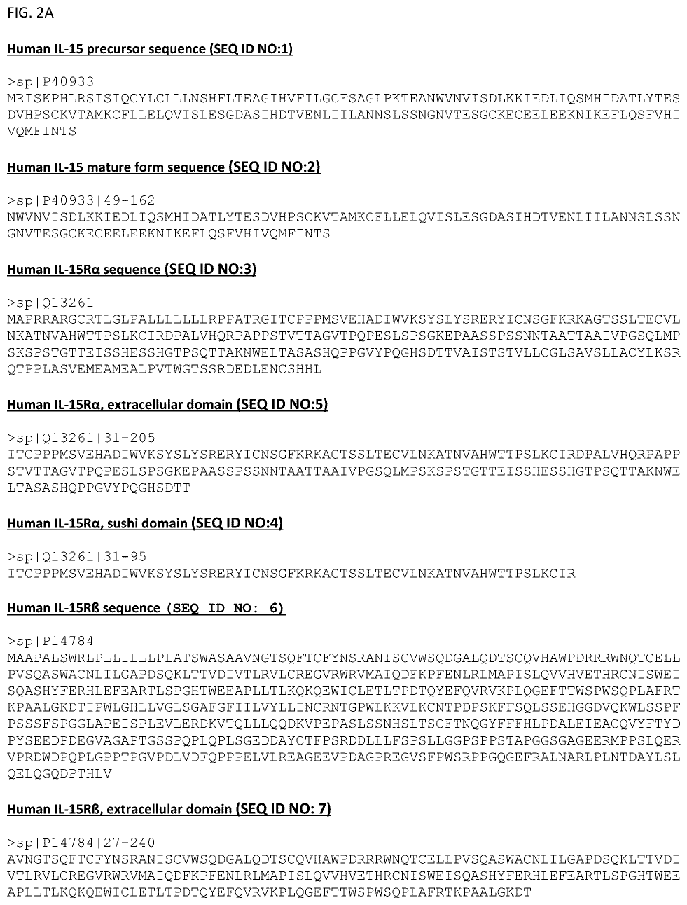

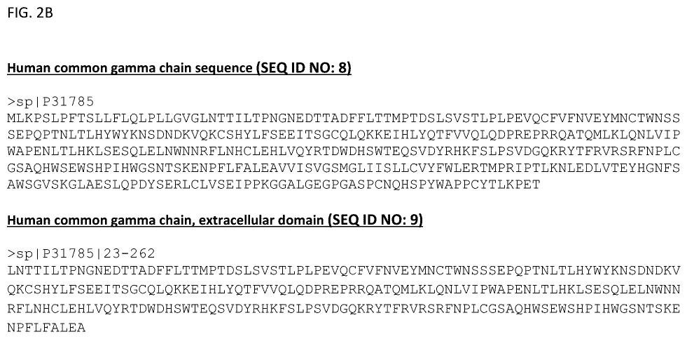

In some embodiments, the human IL-15 protein has the amino acid sequence set forth in NCBI Ref Seq. No. NP_000576.1 as shown in FIGS. 2A-2B. In some cases, the coding sequence of human IL-15 is set forth in NCBI Ref. Seq. No. NM_000585. An exemplary IL-15 protein of the Fc fusion heterodimeric protein outlined herein can have the amino acid sequence of SEQ ID NO:2 or amino acids 49-162 of SEQ ID NO:1. In some embodiments, the IL-15 protein has at least 90%, e.g., 90%, 91%, 92%, 93%, 94%, 95%, 96%, 97%, 98%, 99%, or more sequence identity to SEQ ID NO:2. In some embodiments, the IL-15 protein has the amino acid sequence set forth in SEQ ID NO:2 except with the amino acid substitution N72D. In other embodiments, the IL-15 protein has the amino acid sequence of SEQ ID NO:2 except with one or more amino acid substitutions selected from the group consisting of C42S, L45C, Q48C, V49C, L52C, E53C, E87C, and E89C. In some aspects, the IL-15 protein has one or more amino acid substitutions selected from the group consisting of N1D, N4D, D8N, D30N, D61N, E64Q, N65D, N71Q, N79Q, Q108E, and N112Q. In other embodiments, the amino acid substitutions are N4D/N65D. In certain embodiments, the amino acid substitutions are D30N/N65D. In some embodiments, the amino acid substitution is Q108E. In certain embodiments, the amino acid substitution is N65D. In other embodiments, the amino acid substitutions are D30N/E64Q/N65D. In certain embodiments, the amino acid substitution is N65D. In some instances, the amino acid substitutions are N1D/N65D. In some instances, the amino acid substitutions are D30N/N65D. In some embodiments, the amino acid substitutions are D30N/E64Q/N65D/N71Q/N79Q/N112Q. In some embodiments, the amino acid substitutions are N1D/N4D/D30N/E64Q/N65D/N71Q/N79Q/N112Q. In some embodiments, the amino acid substitutions are N1D/N4D/D30N/E64Q/N65D. In some embodiments, the amino acid substitutions are N4D/D30N/E64Q/N65D. In some embodiments, the amino acid substitutions are D30N/D61N/E64Q/N65D/N71Q/N79Q/N112Q. In some embodiments, the amino acid substitutions are D30N/D61N/N65D/N71Q/N79Q/N112Q. In some embodiments, the amino acid substitutions are D8N/D30N/E64Q/N65D/N71Q/N79Q/N112Q112Q. Optionally, the IL-15 protein also has an N72D substitution. The IL-15 protein of the Fc fusion protein can have 1, 2, 3, 4, 5, 6, 7, 8 or 9 amino acid substitutions. In some embodiments, the IL-15 protein of the Fc fusion protein comprises a D30N substitution. In some embodiments, the IL-15 protein of the Fc fusion protein comprises a N65D substitution. In some embodiments, the IL-15 protein of the Fc fusion contains one or more amino acid substitutions at the IL-15:CD132 interface. In certain embodiments, the Fc fusion protein described herein induces proliferation of NK cells and CD8+ T cells. Additionally, IL-15 variants that can be used with the subject targeted IL-15/IL-15Rα heterodimer proteins are included in FIG. 16.

In some embodiments, the human IL-15 receptor alpha (IL-15Rα) protein has the amino acid sequence set forth in NCBI Ref. Seq. No. NP_002180.1 or SEQ ID NO:3. In some cases, the coding sequence of human IL-15Rα is set forth in NCBI Ref Seq. No. NM_002189.3. An exemplary the IL-15Rα protein of the Fc fusion heterodimeric protein outlined herein can comprise or consist of the sushi domain of SEQ ID NO:3 (e.g., amino acids 31-95 of SEQ ID NO:3), or in other words, the amino acid sequence of SEQ ID NO:4. In some embodiments, the IL-15Rα protein has the amino acid sequence of SEQ ID NO:4 and an amino acid insertion selected from the group consisting of D96, P97, A98, D96/P97, D96/C97, D96/P97/A98, D96/P97/C98, and D96/C97/A98, wherein the amino acid position is relative to full-length human IL-15Rα protein or SEQ ID NO:3. For instance, amino acid(s) such as D (e.g., Asp), P (e.g., Pro), A (e.g., Ala), DP (e.g., Asp-Pro), DC (e.g., Asp-Cys), DPA (e.g., Asp-Pro-Ala), DPC (e.g., Asp-Pro-Cys), or DCA (e.g., Asp-Cys-Ala) can be added to the C-terminus of the IL-15Rα protein of SEQ ID NO: 4. In some embodiments, the IL-15Rα protein has the amino acid sequence of SEQ ID NO:4 and one or more amino acid substitutions selected from the group consisting of K34C, A37C, G38C, S40C, and L42C, wherein the amino acid position is relative to SEQ ID NO:4. The IL-15Rα protein can have 1, 2, 3, 4, 5, 6, 7, 8 or more amino acid mutations (e.g., substitutions, insertions and/or deletions).

2. IL-15/RA Complexes

As outlined herein, the IL-15 variants and the sushi domain can be complexed in at least three different ways to form an IL-15 complex.

In some embodiments, as shown in FIGS. 17B, for example, the IL-15 protein and the IL-15Rα (sushi) are not covalently attached, but rather are self-assembled through regular ligand-ligand interactions. As is more fully described herein, it can be either the IL-15 domain or the sushi domain that is covalently linked to the Fc domain (generally using an optional domain linker). Again, of particular use in this embodiment are a double variant, N4D/N65D or D30N/N65D, a triple variant, D30N/E64Q/N65D, a quadruple variant, N4D/D30N/E64Q/N65D, a quintuple variant, N1D/N4D/D30N/E64Q/N65D, a sextuple variant, D30N/D61N/N65D/N71Q/N79Q/N112Q or D30N/E64Q/N65D/N71Q/N79Q/N112Q, a septuple variant, D30N/D61N/E64Q/N65D/N71Q/N79Q/N112Q or D8N/D30N/E64Q/N65D/N71Q/N79Q/N112Q112Q, or an octuple variant, N1D/N4D/D30N/E64Q/N65D/N71Q/N79Q/N112Q, used with a wild type sushi domain.

In alternative embodiments, the variant IL-15 can be complexed to the sushi domain using a domain linker, such that they are covalently attached as generally shown in FIG. 17D; this figure depicts the sushi domain as the N-terminal domain, although this can be reversed. Again, of particular use in this embodiment are a double variant, N4D/N65D or D30N/N65D, a triple variant, D30N/E64Q/N65D, a quadruple variant, N4D/D30N/E64Q/N65D, a quintuple variant, N1D/N4D/D30N/E64Q/N65D, a sextuple variant, D30N/D61N/N65D/N71Q/N79Q/N112Q or D30N/E64Q/N65D/N71Q/N79Q/N112Q, a septuple variant, D30N/D61N/E64Q/N65D/N71Q/N79Q/N112Q or D8N/D30N/E64Q/N65D/N71Q/N79Q/N112Q112Q, or an octuple variant, N1D/N4D/D30N/E64Q/N65D/N71Q/N79Q/N112Q, used with a wild type sushi domain. Exemplary domain linkers that can be used to attach the IL-15 variant to the sushi domain are depicted in FIG. 8.

Alternatively, each of the IL-15 and sushi domains can be engineered to contain a cysteine amino acid, that forms a disulfide bond to form the complex as is generally shown in FIGS. 17C, again, with either the IL-15 domain or the sushi domain being covalently attached (using an optional domain linker) to the Fc domain. Again, of particular use in this embodiment are a double variant, N4D/N65D or D30N/N65D, (additionally including an amino acid substitution to cysteine), a triple variant, D30N/E64Q/N65D (additionally including an amino acid substitution to cysteine), a quadruple variant, N4D/D30N/E64Q/N65D (additionally including an amino acid substitution to cysteine), a quintuple variant, N1D/N4D/D30N/E64Q/N65D (additionally including an amino acid substitution to cysteine), a sextuple variant, D30N/D61N/N65D/N71Q/N79Q/N112Q or D30N/E64Q/N65D/N71Q/N79Q/N112Q, (additionally including an amino acid substitution to cysteine), a septuple variant, D30N/D61N/E64Q/N65D/N71Q/N79Q/N112Q or D8N/D30N/E64Q/N65D/N71Q/N79Q/N112Q112Q, (additionally including an amino acid substitution to cysteine), or an octuple variant, N1D/N4D/D30N/E64Q/N65D/N71Q/N79Q/N112Q (additionally including an amino acid substitution to cysteine), used with a sushi domain also comprising an amino acid substitution to provide a cysteine.

Additional particular embodiments are outlined below.

B. Anti-LAG-3 Components

In some embodiments, the heterodimeric fusion proteins provided herein include some antibody components.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to antibodies or antibody fragments (antibody monomers) that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

In addition, many of the monomer sequences herein have at least one the cysteines at position 220 replaced by a serine, to reduce disulfide formation. Specifically included within the sequences herein are one or both of these cysteines replaced (C220S).

Thus, “isotype” as used herein is meant any of the subclasses of immunoglobulins defined by the chemical and antigenic characteristics of their constant regions.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3).

A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| VlCDR1 | 24-34 | 27-38 | 24-34 | 24-34 | 24-34 | 30-36 | 27-38 |
| VlCDR2 | 50-56 | 56-65 | 50-56 | 50-56 | 50-56 | 46-55 | 56-62 |
| VlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)).

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat. As shown herein and described below, the pI variants can be in one or more of the CH regions, as well as the hinge region, discussed below.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230. As noted herein, pI variants can be made in the hinge region as well.

The light chain generally comprises two domains, the variable light domain (containing the light chain CDRs and together with the variable heavy domains forming the Fv region), and a constant light chain region (often referred to as CL or Cκ).

Another region of interest for additional substitutions, outlined herein, is the Fc region.

Thus, the present heterodimeric fusion proteins provided herein include one or more antibody domains. As described herein and known in the art, the heterodimeric antibodies provided herein comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

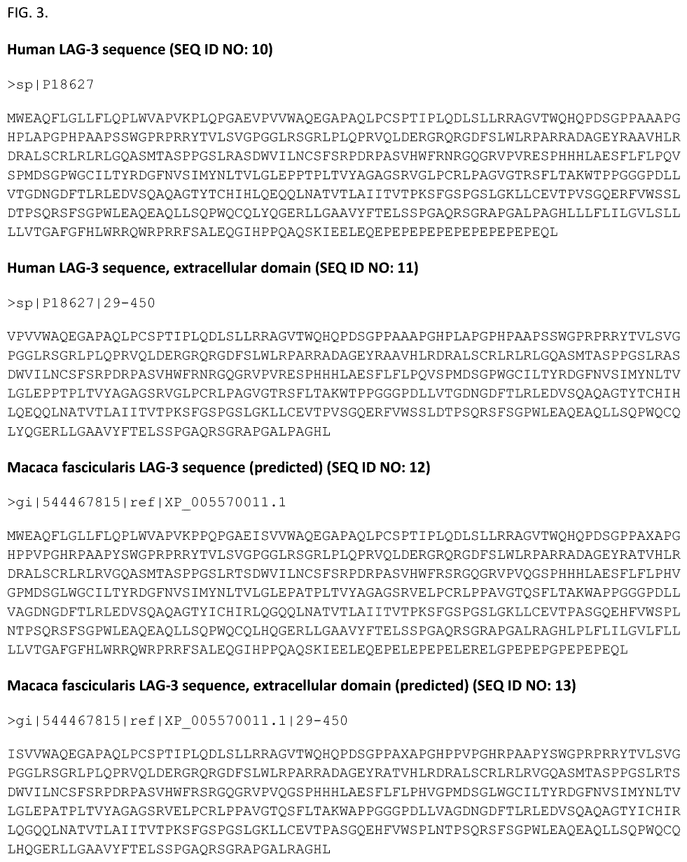

As generally outlined herein, the heterodimeric proteins of the invention include one or more LAG-3 antigen binding domains (e.g., Fvs) that binds human LAG-3. “Lymphocyte-activation gene 3,” “LAG-3,” “LAG3,” “CD223,” and “cluster of differentiation 3,” (e.g., Genebank Accession Numbers NM_002286 and NP_002277 (human)) as used herein is meant a member of an immunoglobulin (Ig) superfamily, that is a type I transmembrane protein with four extracellular Ig-like domains. LAG-3 is a negative regulator of T cells and appears to work in concert with other checkpoint molecules, including CTLA-4 and PD-1. In 1990, Triebel et al. discovered LAG3 as a transmembrane protein expressed by activated human natural killer (NK) and T-cell lines, although it can also be expressed on B cells. The human LAG3 gene has 20% identity with the human CD4 gene, resulting in surface-expressed LAG-3 protein being able to bind major histocompatibility complex (MHC) class II molecules with high affinity. LAG-3 either exists as a cell surface dimer or as a soluble form released by interferon-gamma-producing CD4-positive T cells via proteolytic cleavage of a membrane-proximal connecting peptide. Exemplary LAG-3 sequences are depicted in FIG. 3.

This Fv, or anti-LAG-3 component (the anti-LAG-3 antigen binding domain or LAG-3 ABD) of the subject heterodimer fusion proteins is generally a set of 6 CDRs and/or a variable heavy domain and a variable light domain that form an Fv domain that can bind human LAG-3. As described herein, there are a number of different formats that can be used, generally either by using a scFv or a Fab as outlined herein.

In certain embodiments, the ABDs of the invention comprise a heavy chain variable region with frameworks from a particular germline heavy chain immunoglobulin gene and/or a light chain variable region from a particular germline light chain immunoglobulin gene. For example, such ABDs may comprise or consist of a human ABD comprising heavy or light chain variable regions that are “the product of” or “derived from” a particular germline sequence. An ABD that is “the product of” or “derived from” a human germline immunoglobulin sequence can be identified as such by comparing the amino acid sequence of the ABD to the amino acid sequences of human germline immunoglobulins and selecting the human germline immunoglobulin sequence that is closest in sequence (i.e., greatest % identity) to the sequence of the ABD. An ABD that is “the product of” or “derived from” a particular human germline immunoglobulin sequence may contain amino acid differences as compared to the germline sequence, due to, for example, CDRs, naturally-occurring somatic mutations or intentional introduction of site-directed mutation. However, a humanized ABD typically is at least 90% identical in amino acids sequence to an amino acid sequence encoded by a human germline immunoglobulin gene and contains amino acid residues that identify the ABD as being derived from human sequences when compared to the germline immunoglobulin amino acid sequences of other species (e.g., murine germline sequences). In certain cases, a humanized ABD may be at least 95, 96, 97, 98 or 99%, or even at least 96%, 97%, 98%, or 99% identical in amino acid sequence to the amino acid sequence encoded by the germline immunoglobulin gene. Typically, a humanized ABD derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized ABD may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention). In one embodiment, the parent ABD has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

As shown herein, the anti-LAG-3 ABD can be in the form of either a Fab or an scFv.

In some embodiments, for example as depicted in FIGS. 17B and C, the anti-LAG-3 ABD is a scFv, wherein the VH and VL domains are joined using an scFv linker, which can be optionally a charged scFv linker. As will be appreciated by those in the art, the scFv can be assembled from N- to C-terminus, as N-VH-scFv linker-VL-C or as N-VL-scFv linker-VH-C, with the C terminus of the scFv domain generally being linked to the hinge-CH2-CH3 Fc domain, wherein the hinge in this case serving as a domain linker. Suitable Fvs (including CDR sets and variable heavy/variable light domains) can be used in scFv formats or Fab formats are shown in the Figures as well as disclosed in WO2017/218707, the contents are hereby incorporated in its entirety for all purposes, and in particular for the LAG-3 ABDs in FIG. 11, the data in FIG. 18, FIG. 55, FIG. 56, FIG. 63 and SEQ ID NO:s 36819-36962, SEQ ID NO:s 35417-35606, SEQ ID NO:s 25914-32793 and SEQ ID NO:s 32794-33002 sequences in the sequence listing.

As will further be appreciated by those in the art, all or part of the hinge (which can also be a wild type hinge from IgG1, IgG2 or IgG4 or a variant thereof, such as the IgG4 S241P or S228P hinge variant with the substitution proline at position 228 relative to the parent IgG4 hinge polypeptide (wherein the numbering S228P is according to the EU index and the S241P is the Kabat numbering)) can be used as the domain linker between the scFv and the CH2-CH3 domain, or a different domain linker such as depicted in the Figures can be used.

Alternatively, the LAG-3 ABD can be in the form of a Fab fragment. In this embodiment, the ABD is made up of a variable heavy domain, contributed by a heavy chain, and a variable light domain, contributed by a light chain. Suitable Fvs (including CDR sets and variable heavy/variable light domains) can be used in scFv formats or Fab formats are shown in the Figures as well as disclosed in in WO2017/218707, the contents are hereby incorporated in its entirety for all purposes, and in particular for the LAG-3 ABDs in FIG. 11, the data in FIG. 18, FIG. 55, FIG. 56, FIG. 63 and SEQ ID NO:s 36819-36962, SEQ ID NO:s 35417-35606, SEQ ID NO:s 25914-32793 and SEQ ID NO:s 32794-33002 sequences in the sequence listing.

As will be appreciated by those in the art, suitable LAG-3 binding domains can comprise a set of 6 CDRs as depicted in the sequence listing and figures (e.g., FIGS. 12 and 13), either as they are underlined/bolded or, in the case where a different numbering scheme is used as described herein and as shown in Table 2, as the CDRs that are identified using other alignments within the variable heavy (VH) domain and variable light domain (VL) sequences of those depicted in the figures (e.g., FIGS. 12 and 13A-C) and the sequence listing. Suitable LAG-3 ABDs that find use in the subject targeted IL-15/IL-15Rα heterodimeric fusion proteins can also include the entire VH and VL sequences as depicted in these sequences and figures, used as scFvs or as Fabs.

In one embodiment, the LAG-3 antigen binding domain includes the 6 CDRs (i.e., vhCDR1-3 and vlCDR1-3) of any of the LAG-3 binding domains described in FIGS. 12 and 13A-C or the sequence listing.

In addition to the parental CDR sets disclosed in the figures and sequence listing that form an ABD to LAG-3, provided herein are variant LAG-3 ABDS having CDRs that include at least one modification of the LAG-3 ABD CDRs disclosed herein (e.g., FIGS. 12 and 13A-C). In one embodiment, the heterodimeric fusion protein includes a LAG-3 ABD that includes a set of 6 CDRs with 1, 2, 3, 4, 5, 6, 7, 8, 9, 10 amino acid modifications as compared to the 6 CDRs of a LAG-3 ABD as depicted in FIGS. 12 and 3A-C or the sequence listing. In certain embodiments, the LAG-3 ABD is capable of binding LAG-3 antigen, as measured by at least one of a Biacore, surface plasmon resonance (SPR) and/or BLI (biolayer interferometry, e.g., Octet assay) assay, with the latter finding particular use in many embodiments.

In one embodiment, the LAG-3 ABD of the subject targeted IL-15/IL-15Rα heterodimeric fusion protein includes 6 CDRs that are at least 90, 95, 97, 98 or 99% identical to the 6 CDRs of a LAG-3 ABD as depicted in FIGS. 12 and 13A-C or the sequence listing. In certain embodiments, the LAG-3 ABD is capable of binding to the LAG-3, as measured by at least one of a Biacore, surface plasmon resonance (SPR) and/or BLI (biolayer interferometry, e.g., Octet assay) assay, with the latter finding particular use in many embodiments.

In one embodiment of the subject targeted IL-15/IL-15Rα heterodimeric fusion protein, the LAG-3 antigen binding domain includes the 6 CDRs (i.e., vhCDR1-3 and vlCDR1-3) of one of the following LAG-3 ABDs: 7G8[LAG-3]_H0_L0, 7G8[LAG-3]_H3_L1, 7G8[LAG-3]_H3.30_L1.34. 2A11[LAG-3]_H0_L0, 2A11[LAG-3]_H1_L2, 2A11[LAG-3]_H1.44_L2.142, BMS-986016[LAG-3], IMP731[LAG-3], 13E2[LAG-3], 34F4[LAG-3], IMP761[LAG-3], H5L7[LAG-3], hu22D2[LAG-3], H4sH15482P[LAG-3], L35D4[LAG-3], L35G6[LAG-3], L33H11[LAG-3], L32A9[LAG-3], L32D10[LAG-3], L32A4[LAG-3], L3A1[LAG-3], L3A10[LAG-3], L3C5[LAG-3], and L3E3[LAG-3] (see, e.g., FIGS. 12 and 13A-C). In an exemplary embodiment, the LAG-3 ABD is 7G8[LAG-3]_H3.30_L1.34 or 2A11[LAG-3]_H1.144_L2.142 LAG-3 ABD (see, e.g., FIG. 12).

In one embodiment, the LAG-3 antigen binding domain is a variant LAG-3 antigen binding domain that includes 6 CDRs (i.e., vhCDR1-3 and vlCDR1-3), where the 6 CDRs include 1, 2, 3, 4, 5, 6, 7, 8, 9, or 10 modifications as compared to the 6 CDRs of one of the following LAG-3 ABDs: 7G8[LAG-3]_H0_L0, 7G8[LAG-3]_H3_L1, 7G8[LAG-3]_H3.30_L1.34. 2A11[LAG-3]_H0_L0, 2A11[LAG-3]_H1_L2, 2A11[LAG-3]_H1.44_L2.142, BMS-986016[LAG-3], IMP731[LAG-3], 13E2[LAG-3], 34F4[LAG-3], IMP761[LAG-3], H5L7[LAG-3], hu22D2[LAG-3], H4sH15482P[LAG-3], L35D4[LAG-3], L35G6[LAG-3], L33H11[LAG-3], L32A9[LAG-3], L32D10[LAG-3], L32A4[LAG-3], L3A1[LAG-3], L3A10[LAG-3], L3C5[LAG-3], and L3E3[LAG-3] (see, e.g., FIGS. 12 and 13A-C). In an exemplary embodiment, the LAG-3 ABD is 7G8[LAG-3]_H3.30_L1.34 or 2A11[LAG-3]_H1.144_L2.142 LAG-3 ABD (see, e.g., FIG. 12).

In one embodiment, the LAG-3 antigen binding domain of the IL-15/IL-15Rα heterodimeric fusion protein is a variant LAG-3 antigen binding domain that includes 6 CDRs (i.e., vhCDR1-3 and vlCDR1-3), where the 6 CDRs are at least 90, 95, 97, 98 or 99% identical as compared to the 6 CDRs) of one of the following LAG-3 ABDs: 7G8[LAG-3]_H0_L0, 7G8[LAG-3]_H3_L1, 7G8[LAG-3]_H3.30_L1.34. 2A11[LAG-3]_H0_L0, 2A11[LAG-3]_H1_L2, 2A11[LAG-3]_H1.44_L2.142, BMS-986016[LAG-3], IMP731[LAG-3], 13E2[LAG-3], 34F4[LAG-3], IMP761[LAG-3], H5L7[LAG-3], hu22D2[LAG-3], H4sH15482P[LAG-3], L35D4[LAG-3], L35G6[LAG-3], L33H11[LAG-3], L32A9[LAG-3], L32D10[LAG-3], L32A4[LAG-3], L3A1[LAG-3], L3A10[LAG-3], L3C5[LAG-3], and L3E3[LAG-3] (see, e.g., FIGS. 12 and 13A-C). In an exemplary embodiment, the LAG-3 ABD is 7G8[LAG-3]_H3.30_L1.34 or 2A11[LAG-3]_H1.144_L2.142 LAG-3 ABD (see, e.g., FIG. 12).

In some embodiments, the LAG-3 ABD of the IL-15/IL-15Rα heterodimeric fusion protein includes the variable heavy domain (VH) and variable light domain (VL) of any of the LAG-ABDs disclosed herein, including, but not limited to those disclosed in FIGS. 12 and 13A-C. In addition to the parental LAG-3 variable heavy and variable light domains disclosed herein, provided herein are subject targeted IL-15/IL-15Rα heterodimeric fusion proteins having one or more LAG-3 ABDs that include a variable heavy domain and/or a variable light domain that are variants of a LAG-3 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a LAG-3 ABD depicted in FIGS. 12, 13A-C, or the sequence listing. In certain embodiments, the LAG-3 ABD is capable of binding to LAG-3, as measured at least one of a Biacore, surface plasmon resonance (SPR) and/or BLI (biolayer interferometry, e.g., Octet assay) assay, with the latter finding particular use in many embodiments.

In one embodiment, the variant VH and/or VL domain of the IL-15/IL-15Rα heterodimeric fusion protein is at least 90, 95, 97, 98 or 99% identical to the VH and/or VL of a LAG-3 ABD as depicted in FIGS. 12 and 13A-C or the sequence listing. In certain embodiments, the LAG-3 ABD is capable of binding to LAG-3, as measured by at least one of a Biacore, surface plasmon resonance (SPR) and/or BLI (biolayer interferometry, e.g., Octet assay) assay, with the latter finding particular use in many embodiments.

In some embodiments, the LAG-3 ABD includes the VH and VL of one of the following LAG-3 ABDs: 7G8[LAG-3]_H0_L0, 7G8[LAG-3]_H3_L1, 7G8[LAG-3]_H3.30_L1.34. 2A11[LAG-3]_H0_L0, 2A11[LAG-3]_H1_L2, 2A11[LAG-3]_H1.44_L2.142, BMS-986016[LAG-3], IMP731[LAG-3], 13E2[LAG-3], 34F4[LAG-3], IMP761[LAG-3], H5L7[LAG-3], hu22D2[LAG-3], H4sH15482P[LAG-3], L35D4[LAG-3], L35G6[LAG-3], L33H11[LAG-3], L32A9[LAG-3], L32D10[LAG-3], L32A4[LAG-3], L3A1[LAG-3], L3A10[LAG-3], L3C5[LAG-3], and L3E3[LAG-3] (see, e.g., FIGS. 12 and 13A-C). In an exemplary embodiment, the LAG-3 ABD is 7G8[LAG-3]_H3.30_L1.34 or 2A11[LAG-3]_H1.144_L2.142 LAG-3 ABD (see, e.g., FIG. 12).

In some embodiments, the LAG-3 ABD includes a VH and VL, where the VH and/or VL includes 1, 2, 3, 4, 5, 6, 7, 8, 9 or 10 amino acid modifications as compared to a VH and/or VL of a 7G8_H3.30_L1.34 or 2A11_H1.144_L2.142 LAG-3 ABD (see, e.g., FIG. 12).

In certain embodiments, the LAG-3 ABD includes a VH and VL, where the VH and VL are at least 90, 95, 97, 98 or 99% identical as compared to a VH and VL of one of the following LAG-3 ABDs: 7G8[LAG-3]_H0_L0, 7G8[LAG-3]_H3_L1, 7G8[LAG-3]_H3.30_L1.34. 2A11[LAG-3]_H0_L0, 2A11[LAG-3]_H1_L2, 2A11[LAG-3]_H1.44_L2.142, BMS-986016[LAG-3], IMP731[LAG-3], 13E2[LAG-3], 34F4[LAG-3], IMP761[LAG-3], H5L7[LAG-3], hu22D2[LAG-3], H4sH15482P[LAG-3], L35D4[LAG-3], L35G6[LAG-3], L33H11[LAG-3], L32A9[LAG-3], L32D10[LAG-3], L32A4[LAG-3], L3A1[LAG-3], L3A10[LAG-3], L3C5[LAG-3], and L3E3[LAG-3] (see, e.g., FIGS. 12 and 13A-C). In an exemplary embodiment, the LAG-3 ABD is 7G8[LAG-3]_H3.30_L1.34 or 2A11[LAG-3]_H1.144_L2.142 LAG-3 ABD (see, e.g., FIG. 12).

C. Fc Domains

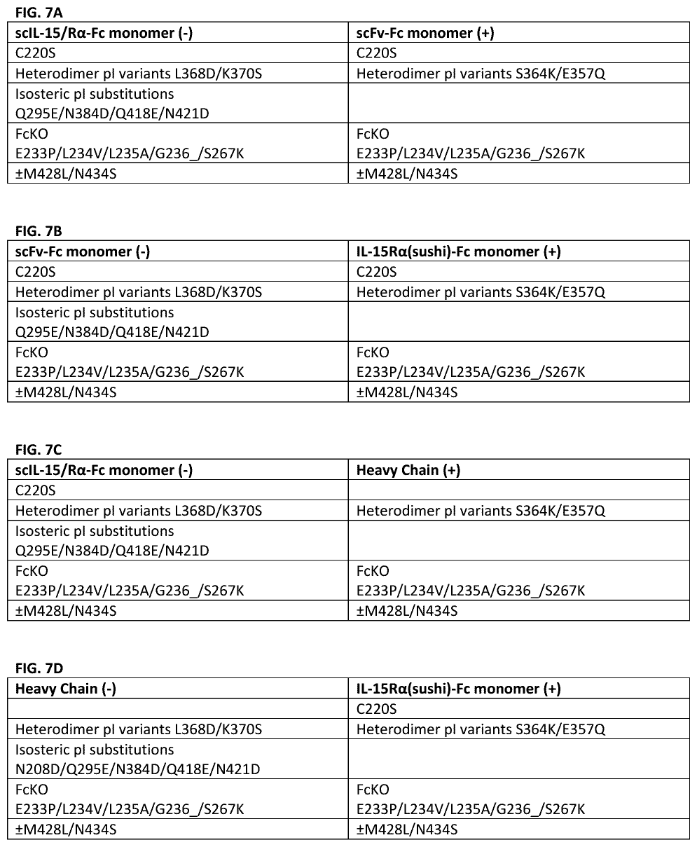

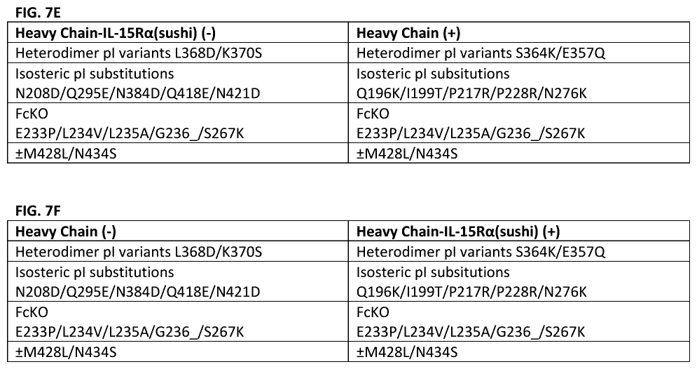

The Fc domain component of the invention is as described herein, which generally contains skew variants and/or optional pI variants and/or ablation variants are outlined herein. See for example the disclosure of WO2017/218707 under the heading “IV Heterodimeric Antibodies”, including sections IV.A, IV.B, IV.C, IV.D, IV.E, IV.F, IV.G, IV.H and IV.I, all of which are expressly incorporated by reference in their entirety. Of particular use in the heterodimeric proteins of the present invention are Fc domains containing “skew variants”, “pI variants”, “ablation variants” and FcRn variants as outlined therein. Particularly useful combinations of such variants are depicted, for example, FIGS. 7A-F.

The Fc domains can be derived from IgG Fc domains, e.g., IgG1, IgG2, IgG3 or IgG4 Fc domains. In an exemplary embodiment, the subject heterodimeric fusion protein provided herein includes an IgG1 Fc domain. The following describes Fc domains that are useful for IL-15/IL-15Rα Fc fusion monomers and anti-LAG-3 antibody fragments of the targeted IL-15/IL-15Rα heterodimeric fusion proteins.

Thus, the “Fc domain” includes the —CH2-CH3 domain, and optionally a hinge domain, and can be from human IgG1, IgG2, IgG3 or IgG4, with Fc domains derived from IgG1. In some of the embodiments herein, when a protein fragment, e.g., IL-15 or IL-15Rα is attached to an Fc domain, it is the C-terminus of the IL-15 or IL-15Rα construct that is attached to all or part of the hinge of the Fc domain. In other embodiments, when a protein fragment, e.g., IL-15 or IL-15Rα, is attached to an Fc domain, it is the C-terminus of the IL-15 or IL-15Rα construct that is attached to the CH1 domain of the Fc domain.

In some of the constructs and sequences outlined herein of an Fc domain protein, the C-terminus of the IL-15 or IL-15Rα protein fragment is attached to the N-terminus of a domain linker, the C-terminus of which is attached to the N-terminus of a constant Fc domain (N-IL-15 or IL-15Rα protein fragment-linker-Fc domain-C) although that can be switched (N-Fc domain-linker-IL-15 or IL-15Rα protein fragment-C). In other constructs and sequence outlined herein, C-terminus of a first protein fragment is attached to the N-terminus of a second protein fragment, optionally via a domain linker, the C-terminus of the second protein fragment is attached to the N-terminus of a constant Fc domain, optionally via a domain linker. In yet other constructs and sequences outlined herein, a constant Fc domain that is not attached to a first protein fragment or a second protein fragment is provided. A heterodimeric fusion protein can contain two or more of the exemplary monomeric Fc domain proteins described herein. Any domain linker can be used to attach a IL-15 or IL-15Rα protein fragment to an Fc domain of the heterodimeric fusion protein provided herein. In some embodiments, the linker is any one of the linkers in FIG. 8.

In some embodiments, the linker is a “domain linker”, used to link any two domains (e.g., IL-15 or IL-15Rα protein fragment to Fc domain or scFv to Fc domain) as outlined herein together, some of which are depicted in FIG. 8. While any suitable linker can be used, many embodiments utilize a glycine-serine polymer or a glycine-alanine polymer, including for example (GS)n, (GSGGS)n (SEQ ID NO: 63), (GGGGS)n (SEQ ID NO: 244), (GGGS)n (SEQ ID NO: 64), (GA)n, (GAGGA)n (SEQ ID NO: 65), (GGGGA)n (SEQ ID NO: 245), and (GGGA)n (SEQ ID NO: 66), where n is an integer of at least one (and generally from 1 to 2 to 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers.

In one embodiment, the heterodimeric fusion proteins contain at least two constant domains which can be engineered to produce heterodimers, such as pI engineering. Other Fc domains that can be used include fragments that contain one or more of the CH1, CH2, CH3, and hinge domains of the invention that have been pI engineered. In particular, the formats depicted in FIG. 17 are heterodimeric fusion proteins, meaning that the protein has two associated Fc sequences self-assembled into a heterodimeric Fc domain and at least one fusion protein (e.g., 1, 2 or more fusion proteins) as more fully described below. In some cases, a first fusion protein is linked to a first Fc and a second fusion protein is linked to a second Fc. In other cases, a first fusion protein is linked to a first Fc, and the first fusion protein is non-covalently attached to a second fusion protein that is not linked to an Fc. In some cases, the heterodimeric fusion protein contains a first fusion protein linked to a second fusion protein which is linked a first Fc sequence, and a second Fc sequence that is not linked to either the first or second fusion proteins.

Accordingly, in some embodiments the present invention provides heterodimeric fusion proteins that rely on the use of two different heavy chain variant Fc sequences, that will self-assemble to form a heterodimeric Fc domain fusion polypeptide.

The present invention is directed to novel constructs to provide heterodimeric fusion proteins that allow binding to one or more binding partners, ligands or receptors. The heterodimeric fusion constructs are based on the self-assembling nature of the two Fc domains of the heavy chains of antibodies, e.g., two “monomers” that assemble into a “dimer”. Heterodimeric Fc fusions are made by altering the amino acid sequence of each monomer as more fully discussed below. Thus, the present invention is generally directed to the creation of heterodimeric fusion proteins which can co-engage binding partner(s) or ligand(s) or receptor(s) in several ways, relying on amino acid variants in the constant regions that are different on each chain to promote heterodimeric formation and/or allow for ease of purification of heterodimers over the homodimers. Specific variants that are included in the Fc domains of specific embodiments of the subject heterodimeric fusion protein are described in greater detail below.

1. Heterodimerization Variants

The present invention provides heterodimeric proteins, including heterodimeric fusion proteins in a variety of formats. Such heterodimeric proteins include two different Fc domains (one on each of the first and second monomers) that include modifications that facilitate the heterodimerization of the first and second monomers and/or allow for ease of purification of heterodimers over homodimers, collectively referred to herein as “heterodimerization variants.” As discussed below, heterodimerization variants can include skew variants (e.g., the “knobs and holes” and “charge pairs” variants described below) as well as “pI variants” that facilitates the separation of homodimers away from heterodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

a. Skew Variants

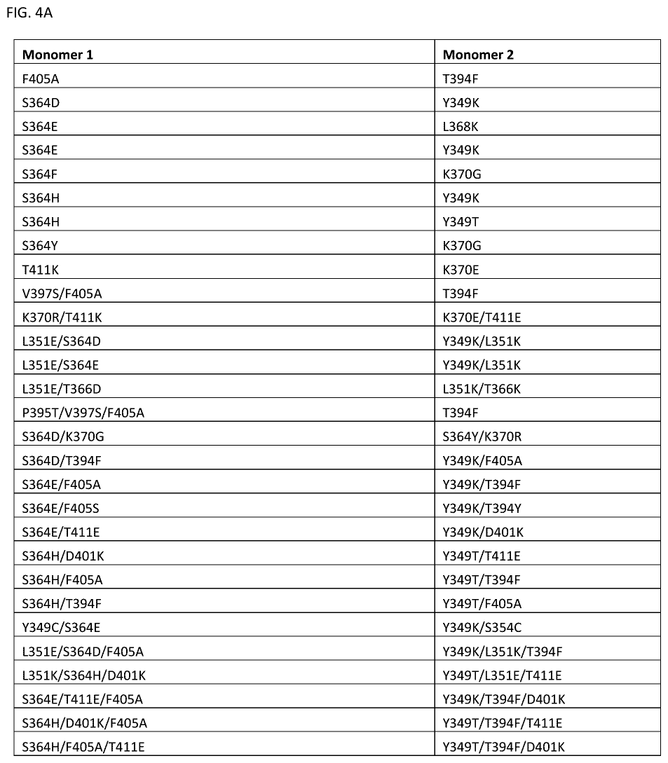

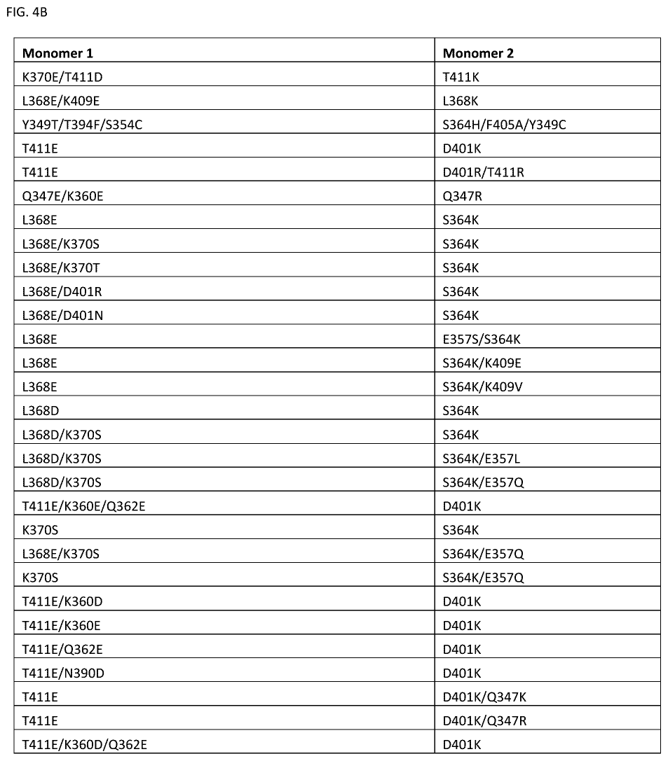

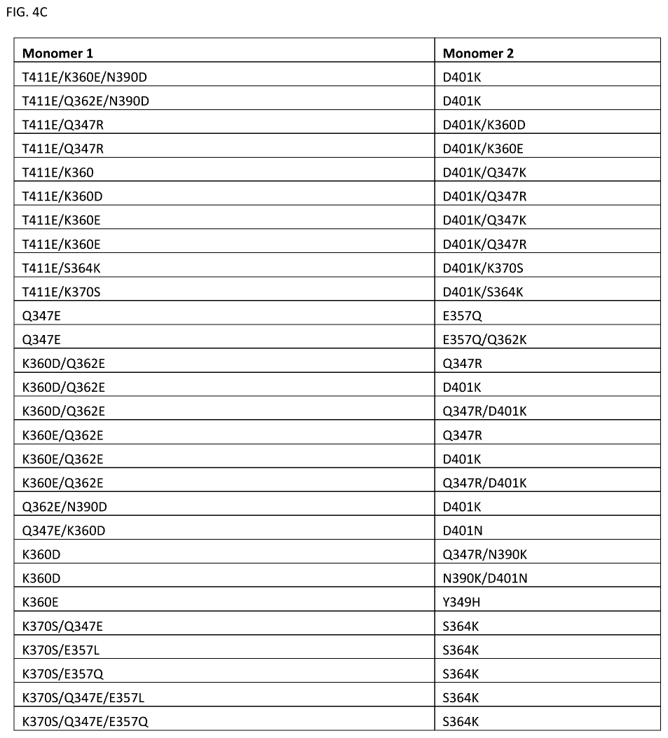

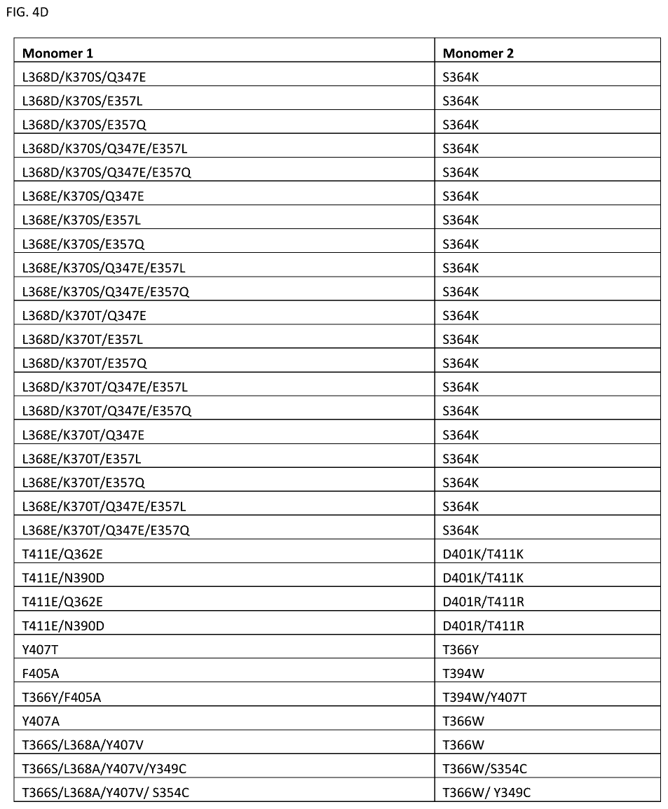

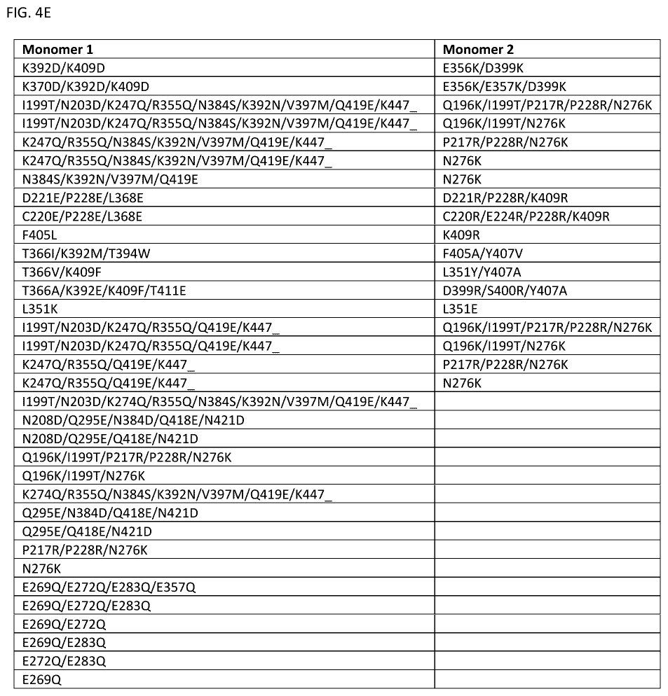

In some embodiments, the subject heterodimeric protein includes skew variants, which are one or more amino acid modifications in a first Fc domain (A) and/or a second Fc domain (B) that favor the formation of Fc heterodimers (Fc dimers that include the first and the second Fc domain; A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIG. 4.

One mechanism for skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

An additional mechanism for skew variants that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants.” These include, but are not limited to, D221E/P228E/L368E paired with D221R/P228R/K409R (e.g., these are “monomer” corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanisms described above. In some embodiments, the heterodimeric protein includes one or more sets of such heterodimerization skew variants. These variants come in “pairs” of “sets.” That is, one set of the pair is incorporated into the first monomer and the other set of the pair is incorporated into the second monomer. Exemplary “skew variants’ in this category include S364K/E357Q: L368D/K370S; L368D/K370S: S364K; L368E/K370S: S364K; T411T/E360E/Q362E D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution sets. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B).

In exemplary embodiments, the heterodimeric fusion protein includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V T366W (optionally including a bridging disulfide, T366S/L368A/Y407V/Y349C: T366W/S354C) “skew” variant amino acid substitution set. In an exemplary embodiment, the heterodimeric fusion protein includes a “S364K/E357Q L368D/K370S” amino acid substitution set.

In some embodiments, the skew variants provided herein are independently incorporated with other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric fusion protein. Further, individual modifications can also independently and optionally be included or excluded from the subject heterodimeric fusion proteins.

b. pI (Isoelectric Point) Variants for Heterodimers

In some embodiments, the heterodimeric fusion protein includes purification variants that advantageously allow for the separation of heterodimeric fusion proteins from homodimeric proteins (“pI variants”).

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

There are several basic mechanisms that can lead to ease of purifying heterodimeric proteins. One such mechanism relies on the use of pI variants which include one or more modifications that affect the isoelectric point of one or both of the monomers of the fusion protein, such that each monomer, and subsequently each dimeric species, has a different pI, thus allowing the isoelectric purification of A-A, A-B and B-B dimeric proteins. Alternatively, some formats also allow separation on the basis of size. As is further outlined above, it is also possible to “skew” the formation of heterodimers over homodimers using skew variants. Thus, a combination of heterodimerization skew variants and pI variants find particular use in the subject heterodimeric fusion proteins provided herein.

Additionally, as more fully outlined below, depending on the format of the heterodimeric fusion protein, pI variants can be either contained within the constant region and/or Fc domains of a monomer, and/or domain linkers can be used. In some embodiments, the heterodimeric fusion protein includes additional modifications for alternative functionalities can also create pI changes, such as Fc, FcRn and KO variants.

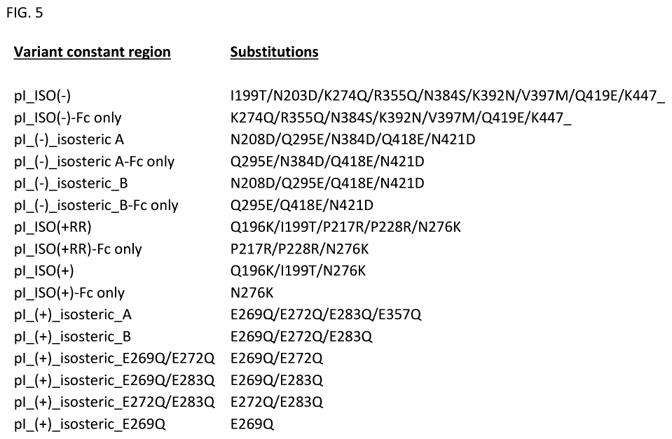

In the embodiments that utilizes pI as a separation mechanism to allow the purification of heterodimeric fusion proteins, amino acid modifications can be introduced into one or both of the monomers of the heterodimeric fusion protein. That is, the pI of one of the monomers (referred to herein for simplicity as “monomer A”) can be engineered away from monomer B, or both monomer A and B can be changed, with the pI of monomer A increasing and the pI of monomer B decreasing. As discussed, the pI changes of either or both monomers can be done by removing or adding a charged residue (e.g., a neutral amino acid is replaced by a positively or negatively charged amino acid residue, e.g., glutamine to glutamic acid), changing a charged residue from positive or negative to the opposite charge (e.g., aspartic acid to lysine) or changing a charged residue to a neutral residue (e.g., loss of a charge; lysine to serine). A number of these variants are shown in the figures, including, FIGS. 4 and 5.

Creating a sufficient change in pI in at least one of the monomers such that heterodimers can be separated from homodimers can be done by using a “wild type” heavy chain constant region and a variant region that has been engineered to either increase or decrease its pI (wt A: B+ or wt A: B−), or by increasing one region and decreasing the other region (A+: B− or A−: B+).

Thus, in general, a component of some embodiments of the present subject fusion proteins are amino acid variants in the Fc domains or constant domain regions that are directed to altering the isoelectric point (pI) of at least one, if not both, of the monomers of a dimeric protein by incorporating amino acid substitutions (“pI variants” or “pI substitutions”) into one or both of the monomers. The separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) of a heterodimeric fusion protein to achieve good separation will depend in part on the starting pI of the components. That is, to determine which monomer to engineer or in which “direction” (e.g., more positive or more negative), the sequences of the Fc domains and any IL-15, IL-15Rα or linker included in each monomer are calculated and a decision is made from there based on the pIs of the monomers. As is known in the art, different Fc domains, linkers IL-15, and IL-15Rα will have different starting pIs. In general, as outlined herein, the pIs are engineered to result in a total pI difference of each monomer of at least about 0.1 logs, with 0.2 to 0.5 being preferred as outlined herein.

In general, as will be appreciated by those in the art, there are two general categories of amino acid modifications that affect pI: those that increase the pI of the protein (basic changes) and those that decrease the pI of the protein (acidic changes). As described herein, all combinations of these variants can be used: one monomer may include a wild type Fc domain, or a variant Fc domain that does not display a significantly different pI from wild-type, and the other monomer includes a Fc domain that is either more basic or more acidic. Alternatively, each monomer may be changed, one to more basic and one to more acidic.

In the case where pI variants are used to achieve heterodimerization, a more modular approach to designing and purifying heterodimeric fusion proteins is provided. Thus, in some embodiments, heterodimerization variants (including skew and pI variants) must be engineered. In addition, in some embodiments, the possibility of immunogenicity resulting from the pI variants is significantly reduced by importing pI variants from different IgG isotypes such that pI is changed without introducing significant immunogenicity (see isotypic variants below). Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g., Asn to Asp; and Gln to Glu).

A side benefit that can occur with this pI engineering is also the extension of serum half-life and increased FcRn binding. That is, as described in US Publ. App. No. US 2012/0028304 (incorporated by reference in its entirety and specifically for the disclosure of pI variants that provide additional function), lowering the pI of antibody constant domains (including those found in Fc fusions) can lead to longer serum retention in vivo. These pI variants for increased serum half-life also facilitate pI changes for purification.

In addition, it should be noted that the pI variants of the heterodimerization variants give an additional benefit for the analytics and quality control process of Fc fusion proteins, as the ability to either eliminate, minimize and distinguish when homodimers are present is significant. Similarly, the ability to reliably test the reproducibility of the heterodimeric fusion protein production is important.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In some embodiments, modifications are made in the hinge of the Fc domain, including positions 208, 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., a heterodimeric fusion protein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447. Exemplary embodiments of pI variants are provided in FIG. 5.

In one embodiment, the heterodimeric fusion protein includes a monomer with a variant Fc domain having pI variant modifications 295E/384D/418E/421D (Q295E/N384D/Q418E/N421D when relative to human IgG1). In one embodiment, the heterodimeric fusion protein includes a monomer with a variant Fc domain having pI variant modifications 208D/295E/384D/418E/421D (N208D/Q295E/N384D/Q418E/N421D when relative to human IgG1). In some embodiments, the heterodimeric fusion protein includes a monomer with a variant Fc domain having pI variant modifications 295E/384D/418E/421D (Q295E/N384D/Q418E/N421D when relative to human IgG1). In one embodiment, the heterodimeric fusion protein includes a monomer with a variant Fc domain having pI variant modifications 196K/199T/217R/228R/276K (Q196K/I199T/P217R/P228R/N276K) when relative to human IgG1).

In one embodiment, the heterodimeric fusion protein includes a monomer with a variant Fc domain having pI variant modifications 217R/228R/276K (P217R/P228R/N276K when relative to human IgG1). Additional exemplary pI variant modification that can be incorporated into the Fc domain of a subject are depicted in FIG. 5.

c. Isotypic Variants

In addition, many embodiments of the invention rely on the “importation” of pI amino acids at particular positions from one IgG isotype into another, thus reducing or eliminating the possibility of unwanted immunogenicity being introduced into the variants. A number of these are shown in FIG. 21 of US Publ. App. No. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant Fc fusion protein. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

d. Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US2014/0370013. As discussed herein, which monomer to engineer is generally decided by the inherent pI of each monomer.

2. Additional Fc Variants for Additional Functionality

In addition to pI amino acid variants, there are a number of useful Fc amino acid modification that can be made for a variety of reasons, including, but not limited to, altering binding to one or more FcγR receptors, altered binding to FcRn receptors, etc.

Accordingly, the proteins of the invention can include amino acid modifications, including the heterodimerization variants outlined herein, which includes the pI variants and steric variants. Each set of variants can be independently and optionally included or excluded from any particular heterodimeric protein.

a. FcγR Variants

Accordingly, there are a number of useful Fc substitutions that can be made to alter binding to one or more of the FcγR receptors. Substitutions that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41), U.S. Ser. Nos. 11/174,287, 11/396,495, 11/538,406, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, amino acid substitutions that increase affinity for FcγRIIc can also be included in the Fc domain variants outlined herein. The substitutions described in, for example, U.S. Ser. Nos. 11/124,620 and 14/578,305 are useful.

In addition, there are additional Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L and 259I/308F/428L.

b. Ablation Variants

Similarly, another category of functional variants are “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific immunomodulatory antibodies desirable to ablate FcγRIIIa binding to eliminate or significantly reduce ADCC activity such that one of the Fc domains comprises one or more Fcγ receptor ablation variants. These ablation variants are depicted in FIG. 31 of U.S. Ser. No. 15/141,350, all of which are herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. It should be noted that the ablation variants referenced herein ablate FcγR binding but generally not FcRn binding.

Exemplary ablation variants are provided in FIG. 5.

c. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

In addition, a monomeric Fc domain can comprise a set of amino acid substitutions that includes C220S/S267K/L368D/K370S or C220S/S267K/S364K/E357Q.

In addition, the heterodimeric fusion proteins can comprise skew variants (e.g., a set of amino acid substitutions as shown in FIGS. 1A-1C of U.S. Ser. No. 15/141,350, all of which are herein incorporated by reference in its entirety), with particularly useful skew variants being selected from the group consisting of S364K/E357Q: L368D/K370S; L368D/K370S S364K; L368E/K370S S364K; T411E/K360E/Q362E: D401K; L368D/K370S: S364K/E357L, K370S S364K/E357Q, T366S/L368A/Y407V T366W and T366S/L368A/Y407V/Y349C: T366W/S354C, optionally ablation variants, optionally charged domain linkers and the heavy chain comprises pI variants.

In some embodiments, the Fc domain comprising an amino acid substitution selected from the group consisting of: 236R, 239D, 239E, 243L, M252Y, V259I, 267D, 267E, 298A, V308F, 328F, 328R, 330L, 332D, 332E, M428L, N434A, N434S, 236R/328R, 239D/332E, M428L, 236R/328F, V259I/V308F, 267E/328F, M428L/N434S, Y436I/M428L, Y436V/M428L, Y436I/N434S, Y436V/N434S, 239D/332E/330L, M252Y/S254T/T256E, V259I/V308F/M428L, E233P/L234V/L235A/G236del/S267K, G236R/L328R and PVA/S267K. In some cases, the Fc domain comprises the amino acid substitution 239D/332E. In other cases, the Fc domain comprises the amino acid substitution G236R/L328R or PVA/S267K.

In one embodiment, a particular combination of skew and pI variants that finds use in the present invention is T366S/L368A/Y407V: T366W (optionally including a bridging disulfide, T366S/L368A/Y407V/Y349C: T366W/S354C) with one monomer comprises Q295E/N384D/Q418E/N481D and the other a positively charged domain linker. As will be appreciated in the art, the “knobs in holes” variants do not change pI, and thus can be used on either monomer. Useful combination of variants that can be used in particular formats of the invention are included in FIGS. 7A-7F.

### III. IL-15/IL-15Rα Fc Fusion×LAG-3 ABD Heterodimeric Proteins

Provided herein are heterodimeric fusion proteins that can bind to the checkpoint inhibitor LAG-3 antigen and can complex with the common gamma chain (yc; CD132) and/or the Il-2 receptor β-chain (IL-2Rβ; CD122). The heterodimeric fusion proteins can contain an IL-15/IL-15Rα-Fc fusion protein and an antibody fusion protein. The IL-15/IL-15Rα-Fc fusion protein can include as IL-15 protein (generally including amino acid substitutions) covalently attached to an IL-15Rα, and an Fc domain. Optionally, the IL-15 protein and IL-15Rα protein are noncovalently attached.

### IV. Useful Formats of the Invention

As shown in FIGS. 17, there are a number of useful formats of the subject targeted IL-15/IL-15Rα heterodimeric fusion proteins. In general, the heterodimeric fusion proteins provided herein have three functional components: an IL-15/IL-15Rα (sushi) component, an anti-LAG-3 component (also referred to as a “LAG-3 binding domain” or “LAG-3 antigen binding domain”), and an Fc component that includes a first Fc domain and second Fc domain, each of which can take different forms as outlined herein and each of which can be combined with the other components in any configuration.

The first and the second Fc domains can have a set of amino acid skew substitutions selected from the following skew variants: a) S267K/L368D/K370S: S267K/S364K/E357Q; b) S364K/E357Q: L368D/K370S; c) L368D/K370S: S364K; d) L368E/K370S: S364K; e) T411E/K360E/Q362E: D401K; f) L368D/K370S: S364K/E357L and g) K370S: S364K/E357Q, according to EU numbering. In an exemplary embodiment, the skew variants are S364K/E357Q: L368D/K370S.

In some embodiments, the first and/or the second Fc domains have an additional set of pI amino acid substitutions selected from the following pI variants: Q295E/N384D/Q418E/N421D, N208/Q295E/N384D/Q418E/N421D or Q196K/I199T/P217R/P228R/N276K, according to EU numbering.

Optionally, the first and/or the second Fc domains have an additional set of ablation (“FcKO”) variants selected from the following FcKO variants: G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to EU numbering.

Optionally, the first and/or second Fc domains have 428L/434S variants for half-life extension.

In embodiments wherein a hinge or partial hinge is used to link an Fc domain to a scFv, IL-15 or IL-15Rα domain, the hinge may optional include a C220S substitution to prevent the hinge from forming undesirable disulfide bonds with any light chains.

Exemplary formats of the subject heterodimeric fusion proteins are provided below.

A. Scil-15/Rα×Scfv

One embodiment is shown in FIGS. 17A, and comprises two monomers. The first monomer comprises, from N- to C-terminus, the IL-15Rα (sushi) domain-(domain linker)-IL-15 variant-(domain linker)-CH2-CH3 (with the second domain linker frequently being a hinge domain), and the second monomer comprises VH-scFv linker-VL-hinge-CH2-CH3 or VL-scFv linker-VH-hinge-CH2-CH3, although in either orientation a domain linker can be substituted for the hinge. This is generally referred to as “scIL-15/Rα×scFv”, with the “sc” standing for “single chain” referring to the attachment of the IL-15 variant and IL-15Rα (sushi) domain using a covalent linker. Preferred combinations of variants for this embodiment are found in FIGS. 17A and B.

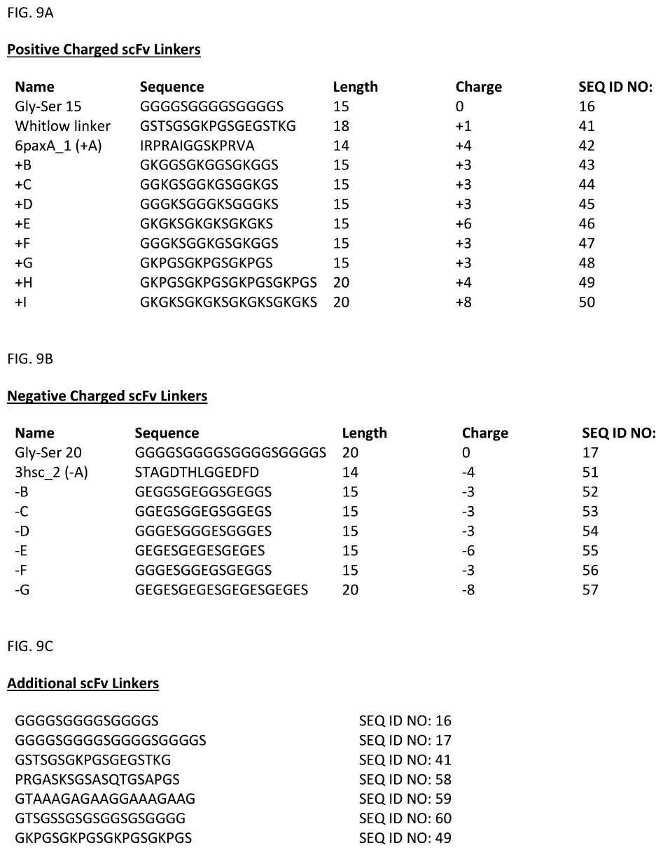

In one embodiment, the targeted IL-15/IL-15Rα heterodimeric protein is an “scIL-15/Rα×scFv” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; and b) a second monomer that includes, from N- to C-terminus, an anti-LAG-3 scFv-(domain linker)-CH2-CH3, where CH2-CH3 is a second Fc domain. Any useful domain linker can be used to attach the various components of the heterodimeric protein including, but not limited to those in FIGS. 8 and 9A-C. In an exemplary embodiment, the domain linkers that attach the IL-15 variant to the first Fc domain and the anti-LAG-3 scFv to the second Fc domain are each antibody hinge domains.

In some embodiments, the anti-LAG-3 scFv includes a variable heavy domain (VH) covalently attached to a variable light domain (VL) by an scFv linker (e.g., FIGS. 9A-C). In one embodiment, the anti-LAG-3 scFv is from N- to C-terminus VH-scFv linker-VL. In another embodiment, the anti-LAG-3 scFv is from N- to C-terminus VL-scFv linker-VH. The C-terminus of the anti-LAG-3 scFv is attached to the N terminus of the first Fc domain by a domain linker (e.g., an antibody hinge domain).

In the scIL-15/Rα×scFv format, one preferred embodiment utilizes an anti-LAG-3 ABD having any of the variable heavy and light domain pairs as shown in FIGS. 12 and 13A-C.

In the scIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first Fc domain; and b) a second monomer that includes, from N- to C-terminus, an anti-LAG-3 scFv-(hinge)-CH2-CH3, where CH2-CH3 is a second Fc domain, and where the anti-LAG-3 scFv includes the variable heavy domain and variable light domain of 7G8_H3.30_L1.34. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first Fc domain; and b) a second monomer that includes, from N- to C-terminus, an anti-LAG-3 scFv-(hinge)-CH2-CH3, where CH2-CH3 is a second Fc domain, and where the anti-LAG-3 scFv includes the variable heavy domain and variable light domain of 2A11_H1.144_L2.142.

In the scIL-15/Rα×scFv format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q112Q. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first Fc domain; and b) a second monomer that includes, from N- to C-terminus, anti-LAG-3 scFv-(hinge)-CH2-CH3, where CH2-CH3 is a second Fc domain, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q112Q. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the scIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first Fc domain; and b) a second monomer that includes, from N- to C-terminus, anti-LAG-3 scFv-(hinge)-CH2-CH3, where CH2-CH3 is a second Fc domain, where the anti-LAG-3 scFv includes the variable heavy domain and variable light domain of 7G8_H3.30_L1.34 or 2A11_H1.144 L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and the scFv includes the variable heavy and light domain pair of 7G8_H3.30_L1.34. In another embodiment of the scIL-15/Rα×scFv format heterodimeric protein, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant is the IL-15 D30N/E64Q/N65D variant and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the scIL-15/Rα×scFv format, one preferred embodiment utilizes the skew variant pair S364K/E357Q: L368D/K370S. In one embodiment, the targeted IL-15/IL-15Rα heterodimeric protein is an “scIL-15/Rα×scFv” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; and b) a second monomer that includes, from N- to C-terminus, anti-LAG-3 scFv-(domain linker)-CH2-CH3, where CH2-CH3 is a second variant Fc domain, and where the first and second variant Fc domains include the skew variant pair S364K/E357Q: L368D/K370S. In an exemplary embodiment, the first variant Fc domain includes skew variants L368D/K370S, and the second variant Fc domain includes skew variants L368D/K370S.

In one embodiment, the targeted IL-15/IL-15Rα heterodimeric protein is an “scIL-15/Rα×scFv” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; and b) a second monomer that includes, from N- to C-terminus, anti-LAG-3 scFv-(domain linker)-CH2-CH3, where CH2-CH3 is a second variant Fc domain, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants L368D/K370S. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the scIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first variant Fc domain; and b) a second monomer that includes, from N- to C-terminus, anti-LAG-3 scFv-(hinge)-CH2-CH3, where CH2-CH3 is a second variant Fc domain, where the anti-LAG-3 scFv includes the variable heavy domain and variable light domain of 7G8_H3.30_L1.34, and where the first and second variant Fc domains include the skew variant pair S364K/E357Q: L368D/K370S. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first Fc domain; and b) a second monomer that includes, from N- to C-terminus, anti-LAG-3 scFv-(hinge)-CH2-CH3, where CH2-CH3 is a second Fc domain, and where the anti-LAG-3 scFv includes the variable heavy domain and variable light domain of 2A11_H1.144_L2.142, and where the first and second variant Fc domains include the skew variant pair S364K/E357Q: L368D/K370S. In an exemplary embodiment, the first variant Fc domain includes skew variants L368D/K370S, and the second variant Fc domain includes skew variants L368D/K370S.

In the scIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the “scIL-15/Rα×scFv” format heterodimeric protein includes: a) a first monomer that includes, from N- to C-terminus, an IL-15Rα (sushi) domain-(domain linker)-IL-15 variant-(hinge)-CH2-CH3, where CH2-CH3 is a first variant Fc domain; and b) a second monomer that includes, from N- to C-terminus, anti-LAG-3 scFv-(hinge)-CH2-CH3, where CH2-CH3 is a second variant Fc domain, where the anti-LAG-3 scFv includes the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants L368D/K370S. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant is the IL-15 D30N/E64Q/N65D variant and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the scIL-15/Rα×scFv format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the “scIL-15/Rα×scFv” format heterodimeric protein includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; and b) a second monomer that includes, from N- to C-terminus, anti-LAG-3 scFv-(hinge)-CH2-CH3, where CH2-CH3 is a second variant Fc domain; where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first and second monomers also each include amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the LAG-3 scFv includes the VH and VL of any of the LAG-3 ABDs in FIGS. 12 and 13A-C. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the scIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17A format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the “scIL-15/Rα×scFv” format heterodimeric protein includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; and b) a second monomer that includes, from N- to C-terminus, anti-LAG-3 scFv-(hinge)-CH2-CH3, where CH2-CH3 is a second variant Fc domain; where the anti-LAG-3 scFv includes the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first and second monomers also each include amino acid substitution C220S. In certain embodiments, the first and second variant Fc domains each further include half-life extension variants M428L/N434S. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

B. scFv×ncIL-15/Rα

This embodiment is shown in FIGS. 17B, and comprises three monomers. The first monomer comprises, from N- to C-terminus, the IL-15Rα (sushi) domain-domain linker-CH2-CH3, and the second monomer comprises VH-scFv linker-VL-hinge-CH2-CH3 or VL-scFv linker-VH-hinge-CH2-CH3, although in either orientation a domain linker can be substituted for the hinge. The third monomer is the variant IL-15 domain. This is generally referred to as “ncIL-15/Rα×scFv” or “scFv×ncIL-15/Rα” with the “nc” standing for “non-covalent” referring to the self-assembling non-covalent attachment of the IL-15 variant and IL-15Rα (sushi) domain.

In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain; and c) an IL-15 variant, where the IL-15 variant and the IL-15Rα (sushi) domain form an IL-15 complex. Any useful domain linker can be used to attach the various components of the heterodimeric protein including, but not limited to those in FIGS. 8 and 9A-C. In an exemplary embodiment, the domain linkers that attach the anti-LAG-3 scFv to the first Fc domain and the IL-15Rα (sushi) domain to the second Fc domain are each antibody hinge domains.

In some embodiments, the anti-LAG-3 scFv includes a variable heavy domain (VH) covalently attached to a variable light domain (VL) by an scFv linker (e.g., FIGS. 9A-C). In one embodiment, the anti-LAG-3 scFv is, from N- to C-terminus, VH-scFv linker-VL. In another embodiment, the anti-LAG-3 scFv is from, N- to C-terminus, VL-scFv linker-VH. The C-terminus of the anti-LAG-3 scFv is attached to the N terminus of the first Fc domain by a domain linker (e.g., an antibody hinge domain).

In the ncIL-15/Rα×scFv format, one preferred embodiment utilizes an anti-LAG-3 ABD having any of the variable heavy and light domain pairs as shown in FIGS. 12 and 13A-C.

In the ncIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain; and c) an IL-15 variant, where the IL-15 variant and the IL-15Rα (sushi) domain form an IL-15 complex, and where the anti-LAG-3 scFv includes the variable heavy domain and variable light domain of 7G8_H3.30_L1.34. In another embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain; and c) an IL-15 variant, where the IL-15 variant and the IL-15Rα (sushi) domain form an IL-15 complex, and where the anti-LAG-3 scFv includes the variable heavy domain and variable light domain of 2A11_H1.144_L2.142.

In the ncIL-15/Rα×scFv format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q112Q. In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain; and c) an IL-15 variant, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q112Q. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the ncIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain; and c) an IL-15 variant, where the IL-15 variant and the IL-15Rα (sushi) domain form an IL-15 complex, where the anti-LAG-3 scFv includes the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the ncIL-15/Rα×scFv format, one preferred embodiment utilizes the skew variant pair S364K/E357Q: L368D/K370S. In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q.

In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the ncIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the anti-LAG-3 scFv includes the variable heavy domain and variable light domain of 7G8_H3.30_L1.34, and where the first and second variant Fc domains include the skew variant pair S364K/E357Q: L368D/K370S. In another embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the anti-LAG-3 scFv includes the variable heavy domain and variable light domain of 2A11_H1.144_L2.142, and where the first and second variant Fc domains include the skew variant pair S364K/E357Q: L368D/K370S.

In the ncIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In an exemplary embodiment, the first variant Fc domain includes skew variants L368D/K370S, and the second variant Fc domain includes skew variants L368D/K370S. In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the anti-LAG-3 scFv includes the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In a particular embodiment, the IL-15 variant includes amino acid substitutions N4D/N65D and the scFv includes the variable heavy and light domain pair of 7G8_H3.30 L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the ncIL-15/Rα×scFv format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first and second monomers also each include amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the LAG-3 scFv includes the VH and VL of any of the LAG-3 ABDs in FIGS. 12 and 13A-C. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the ncIL-15/Rα×scFv format, one preferred embodiment utilizes an anti-LAG-3 ABD having the variable heavy and light domain pair of 7G8_H3.30_L1. or the variable heavy and light domain pair 2A11_H1.144_L2.142 as shown in FIG. 12 with the FIG. 17B format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scFv×ncIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; and c) an IL-15 variant, where the IL-15 variant and the IL-15Rα (sushi) domain form an IL-15 complex, where the anti-LAG-3 scFv includes the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first and second monomers also each include amino acid substitution C220S. In certain embodiments, the first and second variant Fc domains each further include half-life extension variants M428L/N434S. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

C. scFv×dsIL-15/Rα

This embodiment is shown in FIGS. 17C, and comprises three monomers. The first monomer comprises, from N- to C-terminus, the IL-15Rα (sushi) domain-domain linker-CH2-CH3, wherein the IL-15Rα (sushi) domain has an engineered cysteine residue and the second monomer comprises VH-scFv linker-VL-hinge-CH2-CH3 or VL-scFv linker-VH-hinge-CH2-CH3, although in either orientation a domain linker can be substituted for the hinge. The third monomer is the variant IL-15 domain, also engineered to have a cysteine variant amino acid, thus allowing a disulfide bridge to form between the IL-15Rα (sushi) domain and the variant IL-15 domain. This is generally referred to as “scFv×dsIL-15/Rα” or “dsIL-15/Rα×scFv”, with the “ds” standing for “disulfide”.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; and c) an IL-15 variant that includes an amino acid substitution for a cysteine residue, and where the cysteine residue on the IL-15 variant and the cysteine residue on the IL-15Rα (sushi) domain form a disulfide bond. Any useful domain linker can be used to attach the various components of the heterodimeric protein including, but not limited to those in FIGS. 8 and 9A-C. In an exemplary embodiment, the domain linkers that attach the anti-LAG-3 scFv to the first Fc domain and the IL-15Rα (sushi) domain to the second Fc domain and are each antibody hinge domains.

Any useful domain linker can be used to attach the various components of the heterodimeric protein including, but not limited to those in FIGS. 8 and 9A-C. In an exemplary embodiment, the domain linkers that attach the anti-LAG-3 scFv to the first Fc domain and the IL-15Rα (sushi) domain to the second Fc domain and are each antibody hinge domains (e.g., an antibody hinge domain).

In some embodiments, the anti-LAG-3 scFv includes a variable heavy domain (VH) covalently attached to a variable light domain (VL) by an scFv linker (e.g., FIGS. 9A-C). In one embodiment, the anti-LAG-3 scFv is from N- to C-terminus VH-scFv linker-VL. In another embodiment, the anti-LAG-3 scFv is from N- to C-terminus VL-scFv linker-VH. The C-terminus of the anti-LAG-3 scFv is attached to the N terminus of the first Fc domain by a domain linker (e.g., an antibody hinge domain).

In the dsIL-15/Rα×scFv format, one preferred embodiment utilizes an anti-LAG-3 ABD having any of the variable heavy and light domain pairs as shown in FIGS. 12 and 13A-C.

In the dsIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, and where the anti-LAG-3 scFv includes the variable heavy domain and variable light domain of 7G8_H3.30_L1.34. In another embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, and where the anti-LAG-3 scFv includes the variable heavy domain and variable light domain of 2A11_H1.144_L2.142.

In the dsIL-15/Rα×scFv format, one preferred embodiment utilizes the IL-15 N4D/N65D variant or the IL-15 D30N/N65D variant or the IL-15 D30N/E64Q/N65D variant, as well as appropriate cysteine substitutions. In on embodiment, the targeted IL-15/IL-15Rα heterodimeric protein is an “scFv×dsIL-15/Rea” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the dsIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant, as well as appropriate cysteine substitutions. In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the anti-LAG-3 scFv includes the variable heavy domain and variable light domain of 7G8_H3.30_L1.34 or 2A11_H1.144 L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the dsIL-15/Rα×scFv format, one preferred embodiment utilizes the skew variant pair S364K/E357Q: L368D/K370S. In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the dsIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the anti-LAG-3 scFv includes the variable heavy domain and variable light domain of 7G8_H3.30_L1.34, and where the first and second variant Fc domains include the skew variant pair S364K/E357Q: L368D/K370S. In another embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the anti-LAG-3 scFv includes the variable heavy domain and variable light domain of 2A11_H1.144_L2.142, and where the first and second variant Fc domains include the skew variant pair S364K/E357Q: L368D/K370S.

In the dsIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant with the appropriate cysteine substitutions.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(domain linker)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the anti-LAG-3 scFv includes the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In a particular embodiment, the IL-15 variant includes amino acid substitutions N4D/N65D and the scFv includes the variable heavy and light domain pair of 7G8_H3.30 L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30 L1.34. In another embodiment, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

In the dsIL-15/Rα×scFv format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(hinge)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first monomer and second monomer also each include amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the LAG-3 scFv includes the VH and VL of any of the LAG-3 ABDs in FIGS. 12 and 13A-C. In an exemplary embodiment, the LAG-3 scFv includes the VH and VL of any of the LAG-3 ABDs in FIGS. 12 and 13A-C and the IL-15 variant includes amino acid substitutions N4D/N65D. In another exemplary embodiment, the LAG-3 scFv includes the VH and VL of any of the LAG-3 ABDs in FIGS. 12 and 13A-C and the IL-15 variant includes amino acid substitutions D30N/N65D. In yet another exemplary embodiment, the LAG-3 scFv includes the VH and VL of any of the LAG-3 ABDs in FIGS. 12 and 13A-C and the IL-15 variant includes amino acid substitutions D30N/E64Q/N65D.

In the dsIL-15/Rα×scFv format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17C format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scFv×dsIL-15/Rα” format heterodimeric protein that includes: a) a first monomer that includes, from N- to C-terminus, an anti-LAG-3 scFv-(hinge)-CH2-CH3, where CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; and c) an IL-15 variant that includes an amino acid substitution for a cysteine residue, where the cysteine residue on the IL-15 variant and the cysteine residue on the IL-15Rα (sushi) domain form a disulfide bond, where the anti-LAG-3 scFv includes the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first monomer and second monomer also each include amino acid substitution C220S. In certain embodiments, the first and second variant Fc domains each further include half-life extension variants M428L/N434S. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

D. scIL-15/Rα×Fab

This embodiment is shown in FIGS. 17D, and comprises three monomers. The first monomer comprises, from N- to C-terminus, the IL-15Rα (sushi) domain-(domain linker)-variant IL-15-domain linker-CH2-CH3 and the second monomer comprises a heavy chain, VH-CH1-hinge-CH2-CH3. The third monomer is a light chain, VL-CL. This is generally referred to as “scIL-15/Rα×Fab”, with the “sc” standing for “single chain”. The scIL-15/Rα×Fab format (see FIG. 17D) comprises IL-15Rα (sushi) fused to a variant IL-15 by a variable length linker (termed “scIL-15/Rα”) which is then fused to the N-terminus of a heterodimeric Fc-region (inclusive of the hinge). The second monomer is a heavy chain, VH-CH1-hinge-CH2-CH3, while a corresponding light chain (the third monomer) is transfected separately so as to form a Fab with the VH.

In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; b) a second monomer that includes, from N- to C-terminus, a VH-CH1-hinge-CH2-CH3, where VH is a variable heavy domain and CH2-CH3 is a second Fc domain, and c) a light chain that includes from, N- to C-terminus, VL-VC, where VL is a variable light domain, where VH and VL form a LAG-3 binding domain. Any useful domain linker can be used to attach the various components of the heterodimeric protein including, but not limited to those in FIGS. 8 and 9A-C. In an exemplary embodiment, the domain linkers that attach the IL-15 variant to the first Fc domain is an antibody hinge domain (e.g., an antibody hinge domain).

In the scIL-15/Rα×Fab format, one preferred embodiment utilizes an anti-LAG-3 ABD having any of the variable heavy and light domain pairs as shown in FIGS. 12 and 13A-C.

In the scIL-15/Rα×Fab format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric fusion protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; b) a second monomer that includes, from N- to C-terminus, a VH-CH1-hinge-CH2-CH3, where VH is a variable heavy domain and CH2-CH3 is a second Fc domain, and c) a light chain that includes from, N- to C-terminus, VL-VC, where VL is a variable light domain, and where VH and VL are the variable heavy domain and variable light domain of 7G8_H3.30_L1.34, respectively. In another embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; b) a second monomer that includes, from N- to C-terminus, a VH-CH1-hinge-CH2-CH3, where VH is a variable heavy domain and CH2-CH3 is a second Fc domain, and c) a light chain that includes from, N- to C-terminus, VL-VC, where VL is a variable light domain, and where VH and VL are the variable heavy domain and variable light domain of 2A11_H1.144_L2.142, respectively.

In the scIL-15/Rα×Fab format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; b) a second monomer that includes, from N- to C-terminus, a VH-CH1-hinge-CH2-CH3, where VH is a variable heavy domain and CH2-CH3 is a second Fc domain, and c) a light chain that includes from, N- to C-terminus, VL-VC, where VL is a variable light domain, wherein the VH and VL form a LAG-3 binding domain, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the scIL-15/Rα×Fab format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first Fc domain; b) a second monomer that includes, from N- to C-terminus, a VH-CH1-hinge-CH2-CH3, where VH is a variable heavy domain and CH2-CH3 is a second Fc domain, and c) a light chain that includes from, N- to C-terminus, VL-VC, where VL is a variable light domain, where VH and VL are the variable heavy domain and variable light domain of 7G8_H3.30_L1.34 or 2A11_H1.144 L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the scIL-15/Rα×Fab format, one preferred embodiment utilizes the skew variant pair S364K/E357Q: L368D/K370S. In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, where VH is a variable heavy domain and CH2-CH3 is a second variant Fc domain, and c) a light chain that includes from, N- to C-terminus, VL-VC, where VL is a variable light domain, where VH and VL form a LAG-3 binding domain, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q.

In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, where VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the scIL-15/Rα×Fab format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, 2A11_H1.144_L2.142 the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, where VH is a variable heavy domain and CH2-CH3 is a second variant Fc domain, and c) a light chain that includes from, N- to C-terminus, VL-VC, where VL is a variable light domain, where VH and VL are the variable heavy domain and variable light domain of 7G8_H3.30_L1.34, respectively, and where the first and second variant Fc domains include the skew variant pair S364K/E357Q: L368D/K370S. In another embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, where VH is a variable heavy domain and CH2-CH3 is a second variant Fc domain, and c) a light chain that includes from, N- to C-terminus, VL-VC, where VL is a variable light domain, where VH and VL are the variable heavy domain and variable light domain of 2A11_H1.144_L2.142, respectively, and where the first and second variant Fc domains include the skew variant pair S364K/E357Q L368D/K370S.

In the scIL-15/Rα×Fab format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the scIL-15/Rα×Fab format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first monomer also includes amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the scIL-15/Rα×Fab format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17D format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL are the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In some embodiments, the hinge of the first monomer also includes amino acid substitution C220S. In certain embodiments, the first and second variant Fc domains each further include half-life extension variants M428L/N434S. In a particular embodiment, the IL-15 variant includes amino acid substitutions N4D/N65D and the scFv includes the variable heavy and light domain pair of 7G8_H3.30_L1.34. In another embodiment, the IL-15 variant includes amino acid substitutions N4D/N65D and the scFv includes the variable heavy and light domain pair of 2A11_H1.144_L2.142. In one embodiment, the IL-15 variant includes amino acid substitutions D30N/N65D and the scFv includes the variable heavy and light domain pair of 7G8_H3.30_L1.34. In another embodiment of the scIL-15/Rα×scFv format heterodimeric protein, the IL-15 variant includes amino acid substitutions D30N/N65D and the scFv includes the variable heavy and light domain pair of 2A11_H1.144_L2.142. In yet another of embodiment, the IL-15 variant includes amino acid substitutions D30N/E64Q/N65D and the scFv includes the variable heavy and light domain pair of 7G8_H3.30_L1.34. In another embodiment, the IL-15 variant includes amino acid substitutions D30N/E64Q/N65D and the scFv includes the variable heavy and light domain pair of 2A11_H1.144_L2.142.

E. Fab×ncIL-15/Rα

This embodiment is shown in FIGS. 17E, and comprises four monomers. The first monomer comprises, from N- to C-terminus, the IL-15Rα (sushi)domain-(domain linker)-CH2-CH3, and the second monomer comprises a heavy chain, VH-CH1-hinge-CH2-CH3. The third monomer is the light chain that includes, from N- to C-terminus, a variable light domain (VL) and a light constant domain (CL). The fourth monomer is a variant IL-15 domain. This is generally referred to as “Fab×ncIL-15/Rα”, with the “nc” standing for “non-covalent” referring to the self-assembling non-covalent attachment of the IL-15 variant and IL-15Rα (sushi)domain.

In one embodiment, the targeted IL-15/IL-15Rα heterodimeric protein is an “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain; c) a third monomer that includes from, N- to C-terminus, VL-VC, where VL is a variable light domain, and d) a fourth monomer comprising an IL-15 variant, where the VH and the VL form a LAG-3 binding domain, and where the IL-15 and IL-15Rα (sushi) domain form an IL-15 complex. Any useful domain linker can be used to attach the various components of the heterodimeric protein including, but not limited to those in FIGS. 8 and 9A-C. In an exemplary embodiment, the domain linkers that attach the IL-15Rα (sushi) domain to the second Fc domain is an antibody hinge domain (e.g., an antibody hinge domain).

In the Fab×ncIL-15/Rα format, one preferred embodiment utilizes an anti-LAG-3 ABD having any of the variable heavy and light domain pairs as shown in FIG. 12.

In the Fab×ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain; c) a third monomer that includes from, N- to C-terminus, VL-VC, where VL is a variable light domain, and d) a fourth monomer comprising an IL-15 variant, where the VH and the VL are the variable heavy domain and variable light domain of 7G8_H3.30_L1.34, respectively, and where the IL-15 and IL-15Rα (sushi) domain form an IL-15 complex. In another embodiment, the targeted IL-15/IL-15Rα heterodimeric protein is an “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain; c) a third monomer that includes from, N- to C-terminus, VL-VC, where VL is a variable light domain, and d) a fourth monomer comprising an IL-15 variant, where the VH and the VL are the variable heavy domain and variable light domain of 2A11_H1.144_L2.142, respectively, and where the IL-15 and IL-15Rα (sushi) domain form an IL-15 complex.

In the Fab×ncIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the Fab×ncIL-15/Rα format, one preferred embodiment utilizes the skew variant pair S364K/E357Q: L368D/K370S. In one embodiment, the targeted IL-15/IL-15Rα heterodimeric protein is an “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q.

In one embodiment, the targeted IL-15/IL-15Rα heterodimeric protein is an “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL are the variable heavy domain and variable light domain of 7G8_H3.30_L1.34, respectively, where the IL-15 and IL-15Rα (sushi) domain form an IL-15 complex and where the first and second variant Fc domains include the skew variant pair S364K/E357Q: L368D/K370S. In another embodiment, the targeted IL-15/IL-15Rα heterodimeric protein is an “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL are the variable heavy domain and variable light domain of 2A11_H1.144_L2.142, respectively, where the IL-15 and IL-15Rα (sushi) domain form an IL-15 complex, and where the first and second variant Fc domains include the skew variant pair S364K/E357Q: L368D/K370S.

In the Fab×ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant.

In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144 L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the Fab×ncIL-15/Rα format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the hinge-first variant Fc domain of the first monomer includes pI variants N208D/Q295E/N384D/Q418E/N421D, and where numbering is according to EU numbering. In some embodiments, the hinge of the second monomer also includes amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17E format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is a “Fab×ncIL-15/Rα” format heterodimeric protein that includes: a) a first monomer that includes, from N- to C-terminus, a VH-CH1-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain; c) a third monomer that includes from, N- to C-terminus, VL-VC, where VL is a variable light domain, and d) a fourth monomer comprising an IL-15 variant, where the VH and the VL form a LAG-3 binding domain, where the IL-15 and IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the hinge-first variant Fc domain of the first monomer includes pI variants N208D/Q295E/N384D/Q418E/N421D, and where numbering is according to EU numbering. In some embodiments, the hinge of the second monomer also includes amino acid substitution C220S. In certain embodiments, the first and second variant Fc domains each further include half-life extension variants M428L/N434S. In a particular embodiment, the IL-15 variant includes amino acid substitutions N4D/N65D and the scFv includes the variable heavy and light domain pair of 7G8_H3.30_L1.34. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

F. Fab×dsIL-15/Rα

This embodiment is shown in FIGS. 17F, and comprises four monomers. The first monomer comprises, from N- to C-terminus, the IL-15Rα (sushi)domain-domain linker-CH2-CH3, wherein the IL-15Rα (sushi)domain has been engineered to contain a cysteine residue, and the second monomer comprises a heavy chain, VH-CH1-hinge-CH2-CH3. The third monomer is a light chain that includes, from N- to C-terminus, a variable light domain (VL) and a constant light domain (CL). The fourth monomer is the variant IL-15 domain, also engineered to have a cysteine residue, such that a disulfide bridge is formed under native cellular conditions. This is generally referred to as “Fab×dsIL-15/Rα”, with the “ds” standing for “disulfide”.

In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, and where the cysteine residue on the IL-15 variant and the cysteine residue on the IL-15Rα (sushi) domain form a disulfide bond. Any useful domain linker can be used to attach the various components of the heterodimeric protein including, but not limited to those in FIGS. 8 and 9A-C. In an exemplary embodiment, the domain linkers that attach the IL-15Rα (sushi) domain to the second Fc domain is an antibody hinge domain (e.g., an antibody hinge domain).

In the Fab×dsIL-15/Rα format, one preferred embodiment utilizes an anti-LAG-3 ABD having any of the variable heavy and light domain pairs as shown in FIG. 12.

In the Fab×dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL are the variable heavy domain and variable light domain of 7G8_H3.30_L1.34, respectively, and where the cysteine residue on the IL-15 variant and the cysteine residue on the IL-15Rα (sushi) domain form a disulfide bond. In another embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL are the variable heavy domain and variable light domain of 2A11_H1.144_L2.142, respectively, and where the cysteine residue on the IL-15 variant and the cysteine residue on the IL-15Rα (sushi) domain form a disulfide bond.

In the Fab×dsIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, with the appropriate cysteine amino acid substitutions. In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant, with the appropriate cysteine amino acid substitutions. In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first Fc domain; b) a second monomer that includes, from N- to C-terminus, an IL-15Rα (sushi) domain-(domain linker)-CH2-CH3, where CH2-CH3 is a second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30 L1.34. In another embodiment, the IL-15 variant includes amino acid substitutions D30N/E64Q/N65D and VH and VL are the variable heavy domain and variable light domain of 2A11_H1.144_L2.142.

In the Fab×dsIL-15/Rα format, one preferred embodiment utilizes the skew variant pair S364K/E357Q: L368D/K370S. In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q.

In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a variant second Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL are the variable heavy domain and variable light domain of 7G8_H3.30_L1.34, respectively, where the cysteine residue on the IL-15 variant and the cysteine residue on the IL-15Rα (sushi) domain form a disulfide bond, and where the first and second variant Fc domains include the skew variant pair S364K/E357Q: L368D/K370S. In another embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL are the variable heavy domain and variable light domain of 2A11_H1.144_L2.142, respectively, where the cysteine residue on the IL-15 variant and the cysteine residue on the IL-15Rα (sushi) domain form a disulfide bond, and where the first and second variant Fc domains include the skew variant pair S364K/E357Q: L368D/K370S.

In the Fab×dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant with appropriate cysteine substitutions. In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In a particular embodiment, the IL-15 variant includes amino acid substitutions N4D/N65D and VH and VL are the variable heavy domain and variable light domain of 7G8_H3.30 L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30 L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the Fab×dsIL-15/Rα format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the hinge-first variant Fc domain of the first monomer includes pI variants N208D/Q295E/N384D/Q418E/N421D, and where numbering is according to EU numbering. In some embodiments, the hinge of the second monomer also includes amino acid substitution C220S. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the Fab×dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17F format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “Fab×dsIL-15/Rα” format heterodimeric protein that includes: a) a first monomer that includes, from N- to C-terminus, a VH-hinge-CH2-CH3, where VH is a variable heavy domain and CH2-CH3 is a first variant Fc domain; b) a second monomer that includes, from N- to C-terminus, an IL-15Rα (sushi) domain-(domain linker)-CH2-CH3, where CH2-CH3 is a second variant Fc domain and the IL-15Rα (sushi) domain includes an amino acid substitution for a cysteine residue; c) a third monomer that includes, from N- to C-terminus, a VL-CL, where VL is a variable light domain; and d) an IL-15 variant that includes an amino acid substitution for a cysteine residue, where the VH and VL form a LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the hinge-first variant Fc domain of the first monomer includes pI variants N208D/Q295E/N384D/Q418E/N421D, and where numbering is according to EU numbering. In some embodiments, the hinge of the second monomer also includes amino acid substitution C220S. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

G. Mab-Scil-15/Rα

This embodiment is shown in FIGS. 17G, and comprises three monomers (although the fusion protein is a tetramer). The first monomer comprises a heavy chain, VH-CH1-hinge-CH2-CH3. The second monomer comprises a heavy chain with a scIL-15 complex, VH-CH1-hinge-CH2-CH3-domain linker-IL-15Rα (sushi)domain-domain linker-IL-15 variant. The third (and fourth) monomer are light chains, VL-CL. This is generally referred to as “mAb-scIL-15/Rα”, with the “sc” standing for “single chain”. This binds the LAG-3 molecule bivalently.

In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, and where the IL-15 variant and the IL-15Rα (sushi) domain form an IL-15 complex. Any useful domain linker can be used to attach the various components of the heterodimeric protein including, but not limited to those in FIGS. 8 and 9A-C.

In the mAb-scIL-15/Rα format, one preferred embodiment utilizes an anti-LAG-3 ABD having any of the variable heavy and light domain pairs as shown in FIG. 12.

In the mAb-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, and where the IL-15 variant and the IL-15Rα (sushi) domain form an IL-15 complex. In another embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, and where the IL-15 variant and the IL-15Rα (sushi) domain form an IL-15 complex.

In the mAb-scIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the mAb-scIL-15/Rα format, one preferred embodiment utilizes the skew variant pair S364K/E357Q: L368D/K370S. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, where the IL-15 variant and the IL-15Rα (sushi) domain form an IL-15 complex, where the IL-15 variant and the IL-15Rα (sushi) domain form an IL-15 complex, and where the first and second variant Fc domains include the skew variant pair S364K/E357Q: L368D/K370S. In another embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, where the IL-15 variant and the IL-15Rα (sushi) domain form an IL-15 complex, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In the mAb-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In the mAb-scIL-15/Rα format, one preferred embodiment utilizes the skew variant set S364K/E357Q L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17G format, the skew variant set S364K/E357Q: L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144 L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

H. mAb-ncIL-15/Rα

This embodiment is shown in FIGS. 17H, and comprises four monomers (although the heterodimeric fusion protein is a pentamer). The first monomer comprises a heavy chain, VH-CH1-hinge-CH2-CH3. The second monomer comprises a heavy chain with an IL-15Rα (sushi) domain: e.g., VH-CH1-hinge-CH2-CH3-domain linker-IL-15Rα (sushi) domain. The third monomer is a variant IL-15 domain. The fourth (and fifth) monomer are light chains, VL-CL. This is generally referred to as “mAb-ncIL-15/Rα”, with the “nc” standing for “non-covalent”. This also binds the LAG-3 bivalently.

In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where CH2-CH3 is a second Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, and where the IL-15 variant and the IL-15Rα (sushi) domain form an IL-15 complex. Any useful domain linker can be used to attach the various components of the heterodimeric protein including, but not limited to those in FIGS. 8 and 9A-C.

In the mAb-ncIL-15/Rα format, one preferred embodiment utilizes an anti-LAG-3 ABD having any of the variable heavy and light domain pairs as shown in FIG. 12.

In the mAb-ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where CH2-CH3 is a second Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer are the variable heavy domain and variable light domain of 7G8_H3.30_L1.34, respectively, where the VH of the second monomer and the VL of the fifth monomer are the variable heavy domain and variable light domain of 7G8_H3.30_L1.34, respectively, and where the IL-15 variant and the IL-15Rα (sushi) domain form an IL-15 complex. In another embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where CH2-CH3 is a second Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer are the variable heavy domain and variable light domain of 2A11_H1.144_L2.142, respectively, where the VH of the second monomer and the VL of the fifth monomer are the variable heavy domain and variable light domain of 2A11_H1.144_L2.142, respectively, and where the IL-15 variant and the IL-15Rα (sushi) domain form an IL-15 complex.

In the mAb-ncIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where CH2-CH3 is a second Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where CH2-CH3 is a second Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the mAb-ncIL-15/Rα format, one preferred embodiment utilizes the skew variant pair S364K/E357Q: L368D/K370S. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In another exemplary embodiment, the first variant Fc domain includes skew variants S364K/E357Q, and the second variant Fc domain includes skew variants L368D/K370S.

In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In another exemplary embodiment, the first variant Fc domain includes skew variants S364K/E357Q, and the second variant Fc domain includes skew variants L368D/K370S. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer are the variable heavy domain and variable light domain of 7G8_H3.30_L1.34, respectively, where the VH of the second monomer and the VL of the fifth monomer are the variable heavy domain and variable light domain of 7G8_H3.30_L1.34, respectively, where the IL-15 variant and the IL-15Rα (sushi) domain form an IL-15 complex, and where the first and second variant Fc domains include the skew variant pair S364K/E357Q: L368D/K370S. In another embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer are the variable heavy domain and variable light domain of 2A11_H1.144_L2.142, respectively, where the VH of the second monomer and the VL of the fifth monomer are the variable heavy domain and variable light domain of 2A11_H1.144_L2.142, respectively, where the IL-15 variant and the IL-15Rα (sushi) domain form an IL-15 complex, and where the first and second variant Fc domains include the skew variant pair S364K/E357Q: L368D/K370S.

In the mAb-ncIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the first variant Fc domain includes skew variants S364K/E357Q, and the second variant Fc domain includes skew variants L368D/K370S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144 L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the mAb-ncIL-15/Rα format, one preferred embodiment utilizes the skew variant set S364K/E357Q L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-ncIL-15/Rα format, one preferred embodiment utilizes an anti-LAG-3 ABD having the variable heavy and light domain pair of 7G8_H3.30_L1.34 or 2A11_H1.144_L2.142 as shown in FIG. 12 with the FIG. 17H format, the skew variant set S364K/E357Q: L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q.

In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30 L1.34 or 2A11_H1.144 L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-ncIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where CH2-CH3 is a second variant Fc domain; c) a third monomer that includes an IL-15 variant; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the IL-15 variant and the IL-15Rα (sushi) domain form an IL-15 complex, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

I. mAb-dsIL-15/Rα

This embodiment is shown in FIG. 17I, and comprises four monomers (although the heterodimeric fusion protein is a pentamer). The first monomer comprises a heavy chain, VH-CH1-hinge-CH2-CH3. The second monomer comprises a heavy chain with an IL-15Rα (sushi) domain: e.g., VH-CH1-hinge-CH2-CH3-domain linker-IL-15Rα (sushi) domain, where the IL-15Rα (sushi) domain has been engineered to contain a cysteine residue. The third monomer is a variant IL-15 domain, which has been engineered to contain a cysteine residue, such that the IL-15 complex is formed under physiological conditions. The fourth (and fifth) monomer are light chains, VL-CL. This is generally referred to as “mAb-dsIL-15/Rα”, with the “ds” standing for “disulfide”, and it binds LAG-3 bivalently.

In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, and where the cysteine residue on the IL-15 variant and the cysteine residue on the IL-15Rα (sushi) domain form a disulfide bond. Any useful domain linker can be used to attach the various components of the heterodimeric protein including, but not limited to those in FIGS. 8 and 9A-C.

In the mAb-dsIL-15/Rα format, one preferred embodiment utilizes an anti-LAG-3 ABD having any of the variable heavy and light domain pairs as shown in FIG. 12.

In the mAb-dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer are the variable heavy domain and variable light domain of 7G8_H3.30_L1.34, respectively, where the VH of the second monomer and the VL of the fifth monomer are the variable heavy domain and variable light domain of 7G8_H3.30_L1.34, respectively, and where the cysteine residue on the IL-15 variant and the cysteine residue on the IL-15Rα (sushi) domain form a disulfide bond. In another embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer are the variable heavy domain and variable light domain of 2A11_H1.144_L2.142, respectively, where the VH of the second monomer and the VL of the fifth monomer are the variable heavy domain and variable light domain of 2A11_H1.144_L2.142, respectively, and where the cysteine residue on the IL-15 variant and the cysteine residue on the IL-15Rα (sushi) domain form a disulfide bond.

In the mAb-dsIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, with the appropriate cysteine amino acid substitutions. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-dsIL-15/Rα format, one preferred embodiment 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant, with the appropriate cysteine amino acid substitutions. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30 L1.34. In another embodiment, the IL-15 variant includes amino acid substitutions D30N/E64Q/N65D and VH and VL are the variable heavy domain and variable light domain of 2A11_H1.144_L2.142.

In the mAb-dsIL-15/Rα format, one preferred embodiment utilizes the skew variant pair S364K/E357Q: L368D/K370S. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second variant Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In another exemplary embodiment, the first variant Fc domain includes skew variants S364K/E357Q, and the second variant Fc domain includes skew variants L368D/K370S.

In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second variant Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In another exemplary embodiment, the first variant Fc domain includes skew variants S364K/E357Q, and the second variant Fc domain includes skew variants L368D/K370S. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second variant Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer are the variable heavy domain and variable light domain of 7G8_H3.30_L1.34, respectively, where the VH of the second monomer and the VL of the fifth monomer are the variable heavy domain and variable light domain of 7G8_H3.30_L1.34, respectively, where the cysteine residue on the IL-15 variant and the cysteine residue on the IL-15Rα (sushi) domain form a disulfide bond, and where the first and second variant Fc domains include the skew variant pair S364K/E357Q: L368D/K370S. In another embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second variant Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer are the variable heavy domain and variable light domain of 2A11_H1.144_L2.142, respectively, where the VH of the second monomer and the VL of the fifth monomer are the variable heavy domain and variable light domain of 2A11_H1.144_L2.142, respectively, where the cysteine residue on the IL-15 variant and the cysteine residue on the IL-15Rα (sushi) domain form a disulfide bond, and where the first and second variant Fc domains include the skew variant pair S364K/E357Q: L368D/K370S.

In the mAb-dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant with appropriate cysteine substitutions. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain where the cysteine residue on the IL-15 variant and the cysteine residue on the IL-15Rα (sushi) domain form a disulfide bond, where VH and VL are the variable heavy domain and variable light domain of 7G8_H3.30 L1.34 or 2A11_H1.144 L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D/K370S, and the second variant Fc domain includes skew variants S364K/E357Q. In an exemplary embodiment, the first variant Fc domain includes skew variants S364K/E357Q, and the second variant Fc domain includes skew variants L368D/K370S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the mAb-dsIL-15/Rα format, one preferred embodiment utilizes the skew variant set S364K/E357Q L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the mAb-dsIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIGS. 17I format, the skew variant set S364K/E357Q. L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q.

In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144 L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144 L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30 L1.34 or 2A11_H1.144 L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “mAb-dsIL-15/Rα” format heterodimeric protein that includes: a) a first monomer that includes, from N- to C-terminus, a VH-hinge-CH2-CH3, where CH2-CH3 is a first Fc domain; b) a second monomer that includes, from N- to C-terminus, a VH-hinge-CH2-CH3-(domain linker)-IL-15Rα (sushi) domain-(domain linker), where the IL-15Rα (sushi) domain includes an amino acid substitution for a cysteine residue and CH2-CH3 is a second Fc domain; c) a third monomer that includes an IL-15 variant that includes an amino acid substitution for a cysteine residue; and d) a fourth and fifth monomer that each include, from N- to C-terminus, a VL-CL, where VL is a variable light domain, where the VH of the first monomer and the VL of the fourth monomer form a first LAG-3 binding domain, where the VH of the second monomer and the VL of the fifth monomer form a second LAG-3 binding domain, where the cysteine residue on the IL-15 variant and the cysteine residue on the IL-15Rα (sushi) domain form a disulfide bond, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

J. Central-IL-15/Rα

This embodiment is shown in FIGS. 17J, and comprises four monomers forming a tetramer. The first monomer comprises a VH-CH1-[optional domain linker]-IL-15 variant-[optional domain linker]-CH2-CH3, with the second optional domain linker sometimes being the hinge domain. The second monomer comprises a VH-CH1-[optional domain linker]-IL-15Rα (sushi) domain-[optional domain linker]-CH2-CH3, with the second optional domain linker sometimes being the hinge domain. The third (and fourth) monomers are light chains, VL-CL. This is generally referred to as “central-IL-15/Rα”.

In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, and where the IL-15 variant and the IL-15Rα (sushi) domain form an IL-15 complex. Any useful domain linker can be used to attach the various components of the heterodimeric protein including, but not limited to those in FIGS. 8 and 9A-C. In an exemplary embodiment, the domain linkers that attach the IL-15 variant to the first Fc domain and the IL-15Rα (sushi) domain to the second Fc domain are each antibody hinge domains.

In the central-IL-15/Rα format, one preferred embodiment utilizes an anti-LAG-3 ABD having any of the variable heavy and light domain pairs as shown in FIG. 12.

In the central-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, and where the IL-15 variant and the IL-15Rα (sushi) domain form an IL-15 complex. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, and where the IL-15 variant and the IL-15Rα (sushi) domain form an IL-15 complex.

In the “central-IL-15/Rα” format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-IL-15/Rα format, one preferred embodiment utilizes an anti-LAG-3 ABD having the variable heavy and light domain pair 7G8_H3.30_L1.34 or the variable heavy and light domain pair 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the central-IL-15/Rα format, one preferred embodiment utilizes the skew variant pair S364K/E357Q: L368D/K370S. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants S364K and E357Q, and the second variant Fc domain includes skew variants L368D and K370S. In another exemplary embodiment, the first variant Fc domain includes skew variants L368D and K370S, and the second variant Fc domain includes skew variants S364K and E357Q.

In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first Fc domain; b) a second monomer that includes, from N- to C-terminus, a VH-(domain linker)-IL-15Rα (sushi) domain-(domain linker)-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants S364K and E357Q, and the second variant Fc domain includes skew variants L368D and K370S. In another exemplary embodiment, the first variant Fc domain includes skew variants L368D and K370S, and the second variant Fc domain includes skew variants S364K and E357Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first variant Fc domain; b) a second monomer that includes, from N- to C-terminus, a VH-(domain linker)-IL-15Rα (sushi) domain-(domain linker)-CH2-CH3, where CH2-CH3 is a second variant Fc domain; and d) a third and fourth monomer that each include from N- to C-terminus, a VL-CL, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, where the IL-15 variant and the IL-15Rα (sushi) domain form an IL-15 complex, and where the first and second variant Fc domains include the skew variant pair S364K/E357Q: L368D/K370S. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first variant Fc domain; b) a second monomer that includes, from N- to C-terminus, a VH-(domain linker)-IL-15Rα (sushi) domain-(domain linker)-CH2-CH3, where CH2-CH3 is a second variant Fc domain; and d) a third and fourth monomer that each include from N- to C-terminus, a VL-CL, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, where the IL-15 variant and the IL-15Rα (sushi) domain form an IL-15 complex, and where the first and second variant Fc domains include the skew variant pair S364K/E357Q: L368D/K370S.

In the central-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/E64Q/N65D variant with appropriate cysteine substitutions. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(domain linker)-CH2-CH3, where CH2-CH3 is a first variant Fc domain; b) a second monomer that includes, from N- to C-terminus, a VH-(domain linker)-IL-15Rα (sushi) domain-(domain linker)-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants S364K and E357Q, and the second variant Fc domain includes skew variants L368D and K370S. In another exemplary embodiment, the first variant Fc domain includes skew variants L368D and K370S, and the second variant Fc domain includes skew variants S364K and E357Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the central-IL-15/Rα format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(hinge)-CH2-CH3, where CH2-CH3 is a first variant Fc domain; b) a second monomer that includes, from N- to C-terminus, a VH-(domain linker)-IL-15Rα (sushi) domain-(hinge)-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the first variant Fc domain includes pI substitutions Q295E/N384D/Q418D/N421D, and where numbering is according to EU numbering. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(hinge)-CH2-CH3, where CH2-CH3 is a first variant Fc domain; b) a second monomer that includes, from N- to C-terminus, a VH-(domain linker)-IL-15Rα (sushi) domain-(hinge)-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the second variant Fc domain of the second monomer includes pI substitutions Q295E/N384D/Q418D/N421D, and where numbering is according to EU numbering. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-IL-15/Rα format, one preferred embodiment utilizes an anti-LAG-3 ABD having the variable heavy and light domain pair of 7G8_H3.30_L1.34 or 2A11_H1.144_L2.142 as shown in FIG. 12 with the FIGS. 17K, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(hinge)-CH2-CH3, where CH2-CH3 is a first variant Fc domain; b) a second monomer that includes, from N- to C-terminus, a VH-(domain linker)-IL-15Rα (sushi) domain-(hinge)-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the first variant Fc domain includes pI substitutions Q295E/N384D/Q418D/N421D, and where numbering is according to EU numbering. In one embodiment, the targeted IL-15/IL-15Rα heterodimeric protein is an “central-IL-15/Rα” format heterodimeric protein that includes: a) a first monomer that includes, from N- to C-terminus, a VH-(domain linker)-IL-15 variant-(hinge)-CH2-CH3, where CH2-CH3 is a first variant Fc domain; b) a second monomer that includes, from N- to C-terminus, a VH-(domain linker)-IL-15Rα (sushi) domain-(hinge)-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the second variant Fc domain of the second monomer includes pI substitutions Q295E/N384D/Q418D/N421D, and where numbering is according to EU numbering. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

K. Central scIL-15/Rα

This embodiment is shown in FIGS. 17K, and comprises four monomers forming a tetramer. The first monomer comprises a VH-CH1-[optional domain linker]-IL-15Rα (sushi) domain-domain linker-IL-15 variant-[optional domain linker]-CH2-CH3, with the second optional domain linker sometimes being the hinge domain. The second monomer comprises a VH-CH1-hinge-CH2-CH3. The third (and fourth) monomers are light chains, VL-CL. This is generally referred to as “central-scIL-15/Rα”, with the “sc” standing for “single chain”.

In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first Fc domain; b) a second monomer that includes, from N- to C-terminus, a VH-hinge-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, and where the IL-15 variant and the IL-15Rα (sushi) domain form an IL-15 complex. Any useful domain linker can be used to attach the various components of the heterodimeric protein including, but not limited to those in FIGS. 8 and 9A-C. In an exemplary embodiment, the domain linker that attaches the IL-15 variant to the first Fc domain is an antibody hinge domain.

In the central-scIL-15/Rα format, one preferred embodiment utilizes an anti-LAG-3 ABD having any of the variable heavy and light domain pairs as shown in FIG. 12.

In the central-scIL-15/Rα format, one preferred embodiment utilizes an anti-LAG-3 ABD having the variable heavy and light domain pair 7G8_H3.30_L1.34 34 or the variable heavy and light domain pair 2A11_H1.144_L2.142 as shown in as shown in FIG. 12.

In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first Fc domain; b) a second monomer that includes, from N- to C-terminus, a VH-hinge-CH2-CH3, where CH2-CH3 is a second Fc domain; and d) a third and fourth monomer that each include from N- to C-terminus, a VL-CL, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, and where the IL-15 variant and the IL-15Rα (sushi) domain form an IL-15 complex. In another embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-(domain linker)-IL-15 variant-CH2-CH3, where CH2-CH3 is a first Fc domain; b) a second monomer that includes, from N- to C-terminus, a VH-hinge-CH2-CH3, where CH2-CH3 is a second Fc domain; and d) a third and fourth monomer that each include from N- to C-terminus, a VL-CL, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, and where the IL-15 variant and the IL-15Rα (sushi) domain form an IL-15 complex.

In the central-scIL-15/Rα format, one preferred embodiment utilizes an IL-15 variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first Fc domain; b) a second monomer that includes, from N- to C-terminus, a VH-hinge-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first Fc domain; b) a second monomer that includes, from N- to C-terminus, a VH-hinge-CH2-CH3, where CH2-CH3 is a second Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the central-scIL-15/Rα format, one preferred embodiment utilizes the skew variant pair S364K/E357Q: L368D/K370S. In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q.

In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, where the IL-15 variant and the IL-15Rα (sushi) domain form an IL-15 complex, and where the first and second variant Fc domains include the skew variant pair S364K/E357Q: L368D/K370S. In another embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q.

In the central-scIL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair L368D/K370S: S364K/E357Q. In an exemplary embodiment, the first variant Fc domain includes skew variants L368D/K370S, and the second variant Fc domain includes skew variants S364K/E357Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30 L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the central-scIL-15/Rα format, one preferred embodiment utilizes the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(hinge)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the central-scIL-15/Rα format, one preferred embodiment utilizes an anti-LAG-3 ABD having the variable heavy and light domain pair of 7G8_H3.30_L1.34 or 2A11_H1.144_L2.142 as shown in FIG. 12 with the FIG. 17K format, the skew variant set S364K/E357Q: L368D/K370S, the pI variants Q295E/N384D/Q418E/N421D, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “central-scIL-15/Rα” format heterodimeric protein that includes: a) a first monomer that includes, from N- to C-terminus, a VH-(domain linker)-IL-15Rα (sushi) domain-(domain linker)-IL-15 variant-(hinge)-CH2-CH3, where CH2-CH3 is a first variant Fc domain; b) a second monomer that includes, from N- to C-terminus, a VH-hinge-CH2-CH3, where CH2-CH3 is a second variant Fc domain; and d) a third and fourth monomer that each include from N- to C-terminus, a VL-CL, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the hinge of the first monomer further includes variant C220S. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

L. Hetero-mAb-IL-15/Rα

This embodiment is shown in FIGS. 17L, and comprises three monomers (although the fusion protein is a tetramer). The first monomer comprises a heavy chain, VH-CH1-hinge-CH2-CH3-domain linker-IL-15Rα (sushi)domain. The second monomer comprises a heavy chain with a scIL-15 complex, VH-CH1-hinge-CH2-CH3-domain linker-IL-15 variant. The third (and fourth) monomer are light chains, VL-CL. This is generally referred to as “hetero-mAb-IL-15/Rα”. This binds the LAG-3 molecule bivalently.

In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, and where the IL-15 variant and the IL-15Rα (sushi) domain form an IL-15 complex. Any useful domain linker can be used to attach the various components of the heterodimeric protein including, but not limited to those in FIGS. 8 and 9A-C.

In the hetero-mAb-IL-15/Rα format, one preferred embodiment utilizes an anti-LAG-3 ABD having any of the variable heavy and light domain pairs as shown in FIG. 12.

In the hetero-mAb-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12.

In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, and where the IL-15 variant and the IL-15Rα (sushi) domain form an IL-15 complex. In another embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, and where the IL-15 variant and the IL-15Rα (sushi) domain form an IL-15 complex.

In the hetero-mAb-IL-15/Rα format, one preferred embodiment utilizes an IL-variant that includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the hetero-mAb-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12, with either the IL-15 N4D/N65D variant or the IL-15 D30N/N65D variant or the IL-15 D30N/E64Q/N65D variant. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144 L2.142, and where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

In the hetero-mAb-IL-15/Rα format, one preferred embodiment utilizes the skew variant pair S364K/E357Q: L368D/K370S. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the hetero-mAb-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer are the variable heavy domain and variable light domain of 7G8_H3.30_L1.34, respectively, where the VH of the second monomer and the VL of the fourth monomer are the variable heavy domain and variable light domain of 7G8_H3.30_L1.34, respectively, where the IL-15 variant and the IL-15Rα (sushi) domain form an IL-15 complex, where the IL-15 variant and the IL-15Rα (sushi) domain form an IL-15 complex, and where the first and second variant Fc domains include the skew variant pair S364K/E357Q: L368D/K370S. In another embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer are the variable heavy domain and variable light domain of 2A11_H1.144_L2.142, respectively, where the VH of the second monomer and the VL of the fourth monomer are the variable heavy domain and variable light domain of 2A11_H1.144_L2.142, respectively, where the IL-15 variant and the IL-15Rα (sushi) domain form an IL-15 complex, where the IL-15 variant and the IL-15Rα (sushi) domain form an IL-15 complex, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In the hetero-mAb-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 and the skew variant pair S364K/E357Q: L368D/K370S with either the IL-15 N4D/N65D variant or the IL-15 D30N/N65D variant or the IL-15 D30N/E64Q/N65D variant. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q, and where the first and second variant Fc domains include the skew variant pair S364K/E357Q: L368D/K370S. In an exemplary embodiment, the first variant Fc domain includes skew variants L368D and K370S, and the second variant Fc domain includes skew variants S364K and E357Q. In a particular embodiment, the IL-15 variant includes amino acid substitutions N4D/N65D and VH and VL are the variable heavy domain and variable light domain of 7G8_H3.30 L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30 L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142. In an exemplary embodiment, the first variant Fc domain includes skew variants L368D and K370S, and the second variant Fc domain includes skew variants S364K and E357Q. In an exemplary embodiment, the first variant Fc domain includes skew variants S364K and E357Q, and the second variant Fc domain includes skew variants L368D and K370S.

In the hetero-mAb-IL-15/Rα format, one preferred embodiment utilizes the skew variant set S364K/E357Q L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

In the hetero-mAb-IL-15/Rα format, one preferred embodiment utilizes an anti-LAG-3 ABD having the variable heavy and light domain pair of 7G8_H3.30_L1.34 or the variable heavy and light domain pair of 2A11_H1.144_L2.142 as shown in FIG. 12 with the FIG. 17L format, the skew variant set S364K/E357Q: L368D/K370S, the pI variants N208D/Q295E/N384D/Q418D/N421D and/or Q196K/I199T/P271R/P228R/N276K, the ablation variants E233P/L234V/L235A/G236_/S267K on both first and second monomers, and optionally the 428L/434S variants on both first and second monomers.

In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D and the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-first variant Fc domain of the first monomer includes pI substitutions N208D/Q295E/N384D/Q418D/N421D, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants L368D/K370S and the second variant Fc domain include the skew variant pair S364K/E357Q, where the first and second variant Fc domains each include FcKO variants E233P/L234V/L235A/G236del/S267K, where the hinge-second variant Fc domain of the second monomer includes pI variants Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K and the hinge-second variant Fc domain of the second monomer includes pI variants N208D/Q295E/N384D/Q418D/N421D, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the first monomer includes pI substitutions Q196K/I199T/P271R/P228R/N276K, and where numbering is according to EU numbering. In one embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first variant Fc domain; b) a second monomer that includes, from N- to C-terminus, a VH-hinge-CH2-CH3-(domain linker)-IL-15 variant, where CH2-CH3 is a second variant Fc domain; and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant and the IL-15Rα (sushi) domain form an IL-15 complex, where VH and VL are the variable heavy domain and variable light domain of 7G8_H3.30_L1.34 or 2A11_H1.144_L2.142, where the first variant Fc domain includes skew variants S364K/E357Q and the second variant Fc domain include the skew variant pair L368D/K370S, where the first and second variant Fc domains each include FcKO variants E233P/L234V/L235A/G236del/S267K, where the hinge-first variant Fc domain of the hinge-second variant Fc domain of the second monomer includes pI variants N208D/Q295E/N384D/Q418D/N421D, and where numbering is according to EU numbering. In certain embodiments, the first and second variant Fc domains each further include half-life extension variants M428L/N434S. In a particular embodiment, the IL-15 variant includes amino acid substitutions N4D/N65D and VH and VL are the variable heavy domain and variable light domain of 7G8_H3.30_L1.34. In another embodiment, the IL-15 variant includes amino acid substitutions N4D/N65D and VH and VL are the variable heavy domain and variable light domain of 2A11_H1.144_L2.142. In one embodiment, the IL-15 variant includes amino acid substitutions D30N/N65D and VH and VL are the variable heavy domain and variable light domain of 7G8_H3.30_L1.34. In another embodiment, the IL-15 variant includes amino acid substitutions D30N/N65D and VH and VL are the variable heavy domain and variable light domain of 2A11_H1.144_L2.142. In yet another of embodiment, the IL-15 variant includes amino acid substitutions D30N/E64Q/N65D and VH and VL are the variable heavy domain and variable light domain of 7G8_H3.30_L1.34. In another embodiment, the IL-15 variant includes amino acid substitutions D30N/E64Q/N65D and VH and VL are the variable heavy domain and variable light domain of 2A11_H1.144_L2.142.

### V. Particularly Useful Embodiments of the Invention

The present invention provides a targeted IL-15/IL-15Rα heterodimeric protein comprising at least two monomers, one of which contains an anti-LAG-3 ABD and the other that contains an IL-15/RA complex, joined using heterodimeric Fc domains.

In some embodiments, the first and the second Fc domains have a set of amino acid substitutions selected from the group consisting of S267K/L368D/K370S: S267K/S364K/E357Q; S364K/E357Q L368D/K370S; L368D/K370S: S364K; L368E/K370S: S364K; T411E/K360E/Q362E: D401K; L368D/K370S: S364K/E357L and K370S: S364K/E357Q, according to EU numbering.

In some instances, the first and/or the second Fc domains have an additional set of amino acid substitutions comprising Q295E/N384D/Q418E/N421D, according to EU numbering. In some cases, the first and/or the second Fc domains have an additional set of amino acid substitutions consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to EU numbering.

In some embodiments, the IL-15 protein has a polypeptide sequence selected from the group consisting of SEQ ID NO:1 (full-length human IL-15) and SEQ ID NO:2 (truncated human IL-15), and the IL-15Rα protein has a polypeptide sequence selected from the group consisting of SEQ ID NO:3 (full-length human IL-15Rα) and SEQ ID NO:4 (sushi domain of human IL-15Rα).

In embodiments the IL-15 protein and the IL-15Rα protein can have a set of amino acid substitutions selected from the group consisting of E87C: D96/P97/C98; E87C: D96/C97/A98; V49C: S40C; L52C: S40C; E89C: K34C; Q48C: G38C; E53C: L42C; C42S: A37C; and L45C: A37C, respectively.

In some embodiments, the IL-15 protein is a variant protein that has a sequence selected from FIG. 16 to reduce potency. In some embodiments, the IL-15 protein is a variant protein having one or more amino acid substitutions at the IL-15:CD132 interface.

In some embodiments, the LAG-3 antigen binding domain comprises an anti-LAG-3 scFv or an anti-LAG-3 Fab. In an exemplary embodiment, the LAG-3 ABD includes the VH and VL of any of the LAG-3 ABDs depicted in FIGS. 12 and 13A-C.

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 D30N/E64Q/N65D/N71Q/N79Q/N112Q variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 N1D/N4D/D30N/E64Q/N65D/N71Q/N79Q/N112Q variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 N1D/N4D/D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 N4D/D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 D30N/D61N/E64Q/N65D/N71Q/N79Q/N112Q variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 D30N/D61N/N65D/N71Q/N79Q/N112Q variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “scIL-15/Rα×Fab” format heterodimeric protein that includes: a) a first monomer that includes, from N- to C-terminus, an IL-15Rα (sushi) domain-(domain linker)-IL-15 variant-(domain linker)-CH2-CH3, where CH2-CH3 is a first variant Fc domain; b) a second monomer that includes, from N- to C-terminus, a VH-CH1-hinge-CH2-CH3, variant here VH is a variable heavy domain and CH2-CH3 is a second variant Fc domain, and c) a light chain that includes from, N- to C-terminus, VL-VC, where VL is a variable light domain, where VH and VL form a LAG-3 binding domain, where the IL-15 variant is an IL-15 D8N/D30N/E64Q/N65D/N71Q/N79Q/N112Q variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain, and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant is an IL-15 D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “mAb-scIL-15/Rα” format heterodimeric protein that includes: a) a first monomer that includes, from N- to C-terminus, a VH-hinge-CH2-CH3, where CH2-CH3 is a first variant Fc domain; b) a second monomer that includes, from N- to C-terminus, a VH-hinge-CH2-CH3-(domain linker)-IL-15Rα (sushi) domain-(domain linker)-IL-15 variant, where CH2-CH3 is a second Fc domain, and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant is an IL-15 N4D/D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

In an exemplary embodiment, the targeted IL-15/IL-15Rα heterodimeric protein is an “hetero-mAb-IL-15/Rα” format heterodimeric protein that includes: a) a first monomer that includes, from N- to C-terminus, a VH-hinge-CH2-CH3-(domain linker)-IL-15Rα (sushi) domain, where CH2-CH3 is a first Fc domain; b) a second monomer that includes, from N- to C-terminus, a VH-hinge-CH2-CH3-(domain linker)-IL-15 variant, where CH2-CH3 is a second Fc domain, and c) a third and fourth monomer that each include, from N- to C-terminus, a VL-CL, where VL is a variable light domain, where the VH of the first monomer and the VL of the third monomer form a first LAG-3 binding domain, where the VH of the second monomer and the VL of the fourth monomer form a second LAG-3 binding domain, where the IL-15 variant is an IL-15 N4D/D30N/E64Q/N65D variant, where the first variant Fc domain includes skew variants L368D/K370S and the second variant Fc domain includes skew variants S364K/E357Q, where the first and second variant Fc domains each include FcKO variants E233P/L234V/L235A/G236del/S267K, where the first variant Fc domain includes pI variants Q295E/N384D/Q418E/N421D, and where numbering is according to EU numbering. In certain embodiments, the first and second variant Fc domains each further include half-life extension variants M428L/N434S. In certain embodiments, the hinge of the first monomer includes also includes amino acid substitution C220S and the first and second variant Fc domains each further include half-life extension variants M428L/N434S. In some embodiments, the VH and VL are the variable heavy domain and variable light domain of any of the LAG-3 ABDs in FIG. 12 or 13A-C. In some embodiments, the VH and VL are the variable heavy domain and variable light domain of 7G8_H3.30_L1.34 or 2A11_H1.144_L2.142 (FIG. 12).

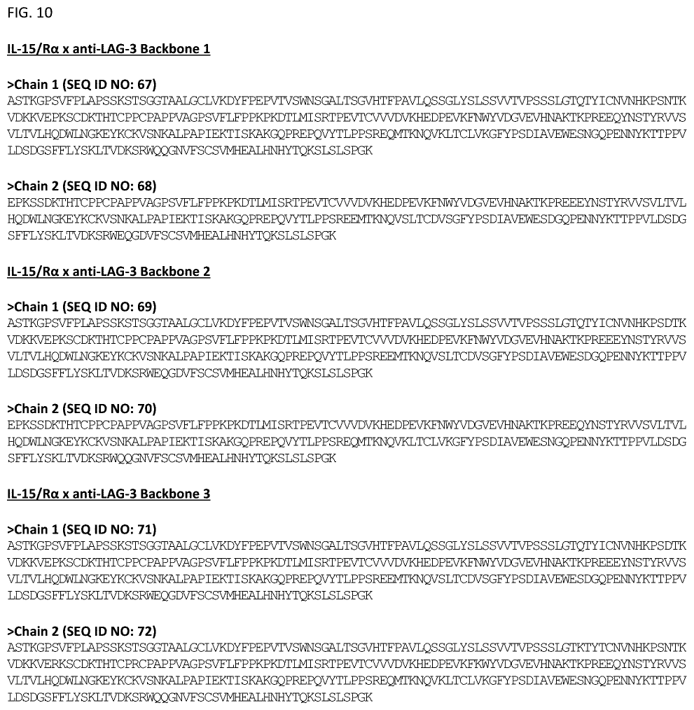

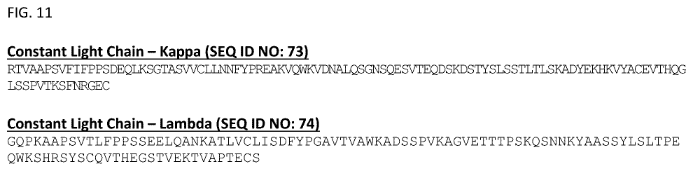

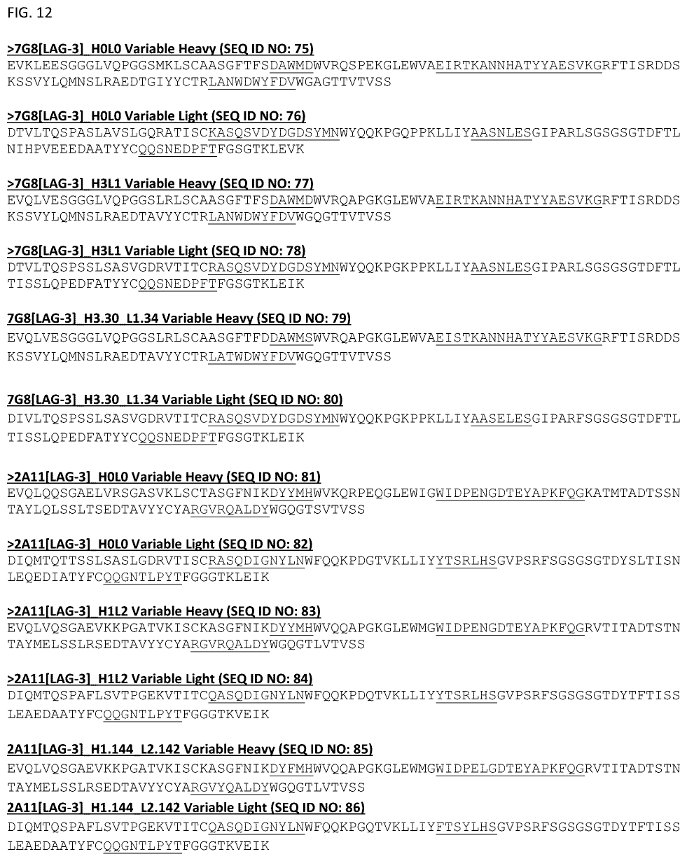

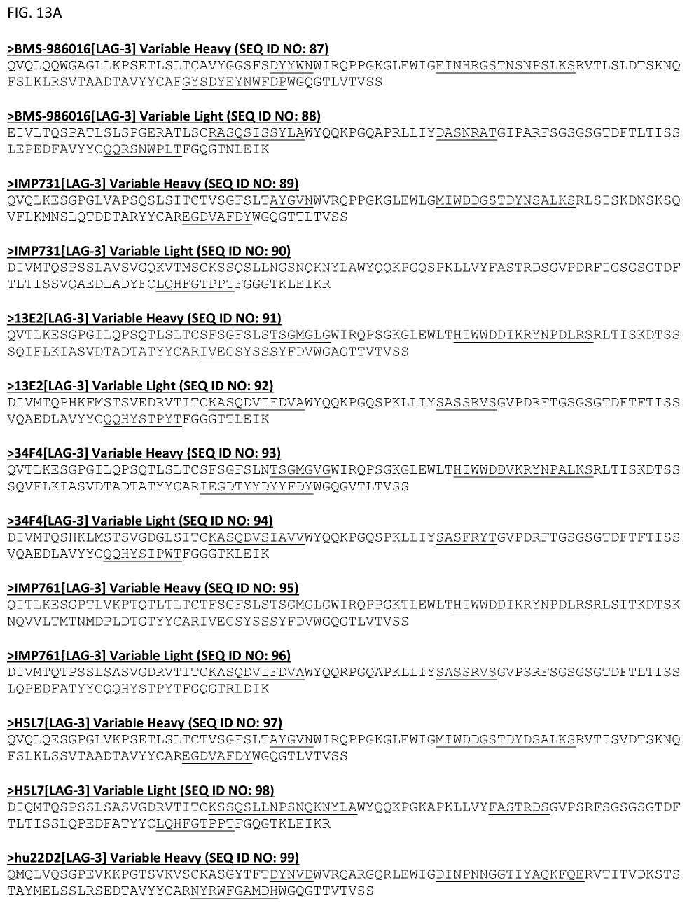

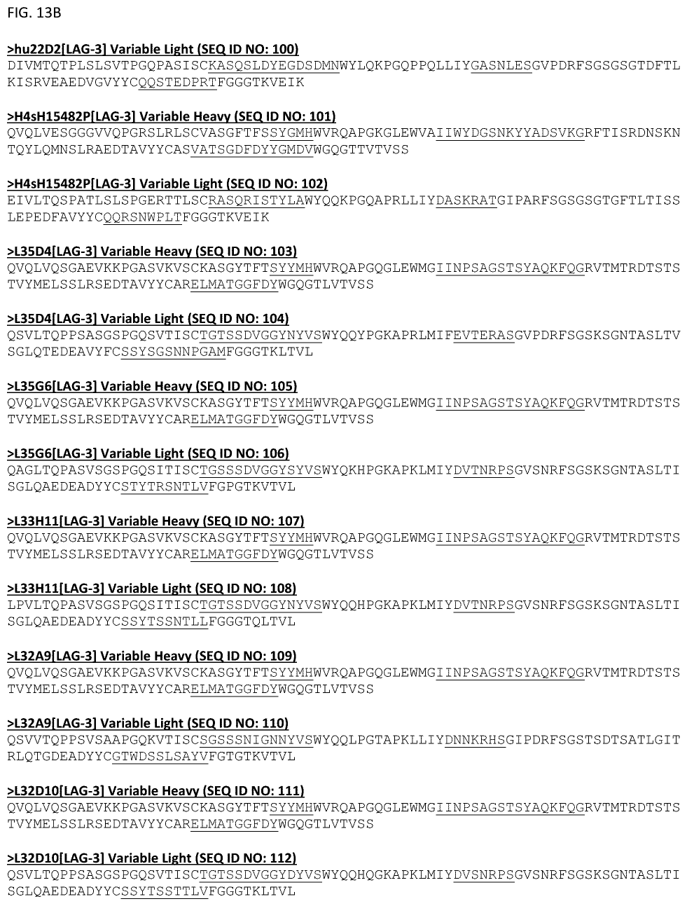

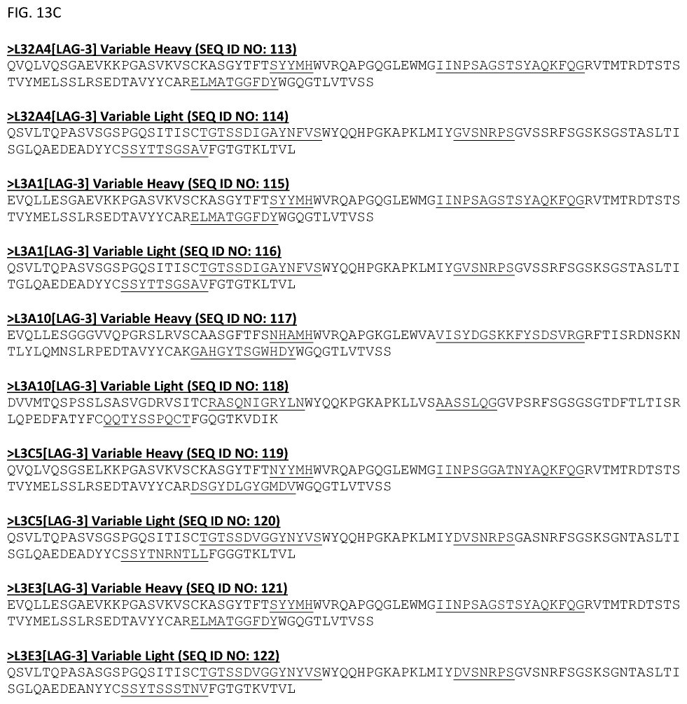

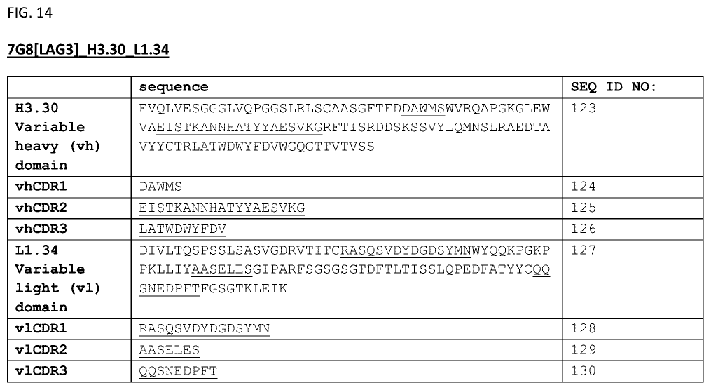

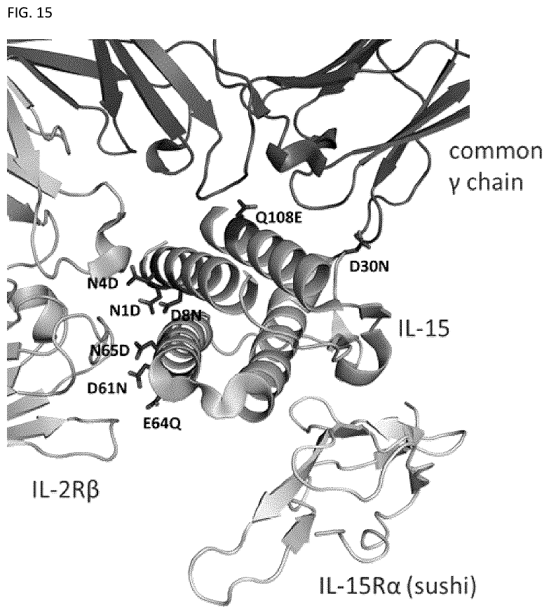

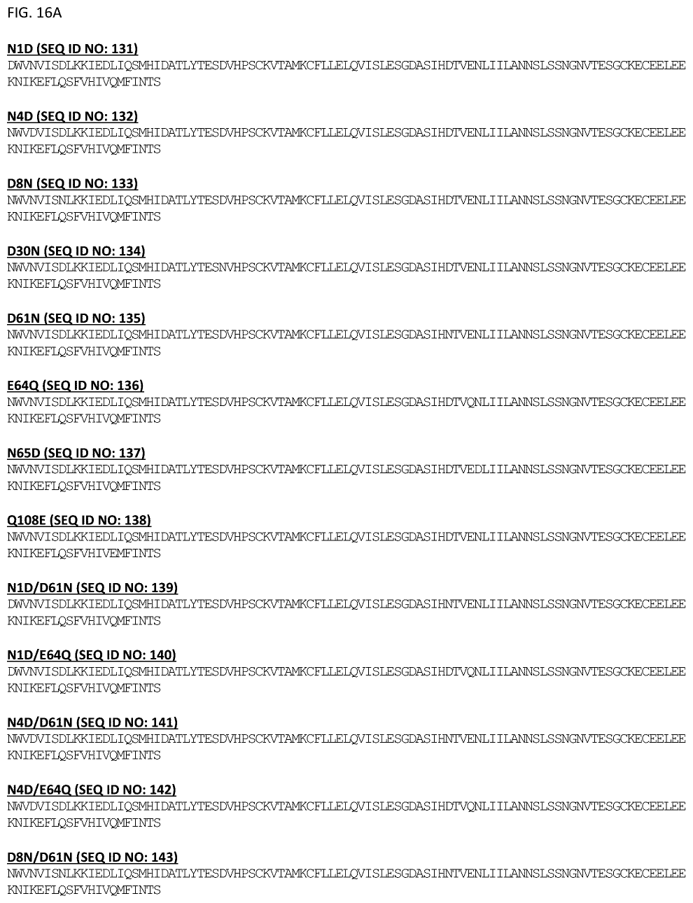

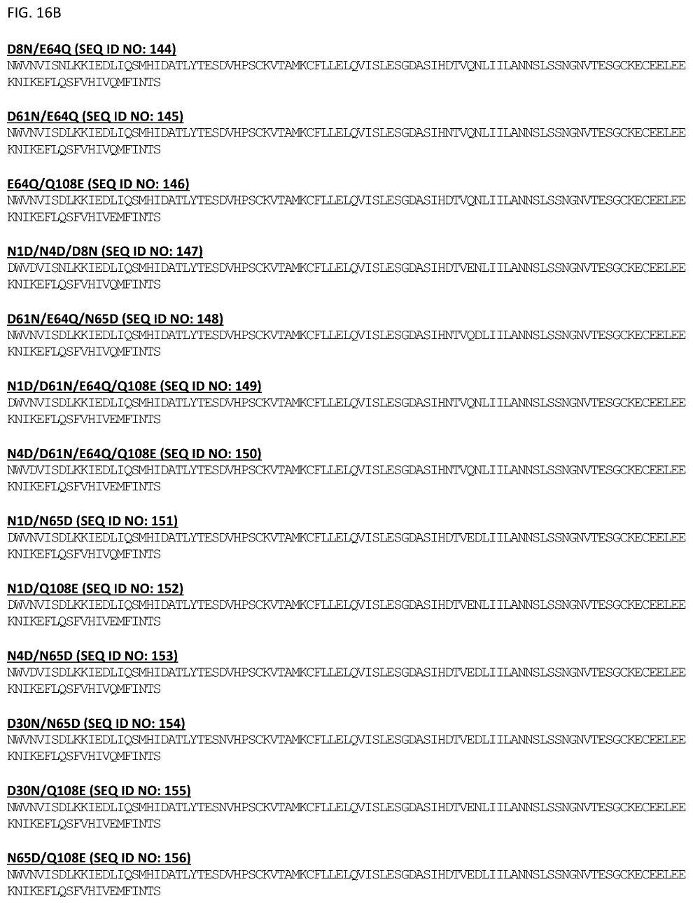

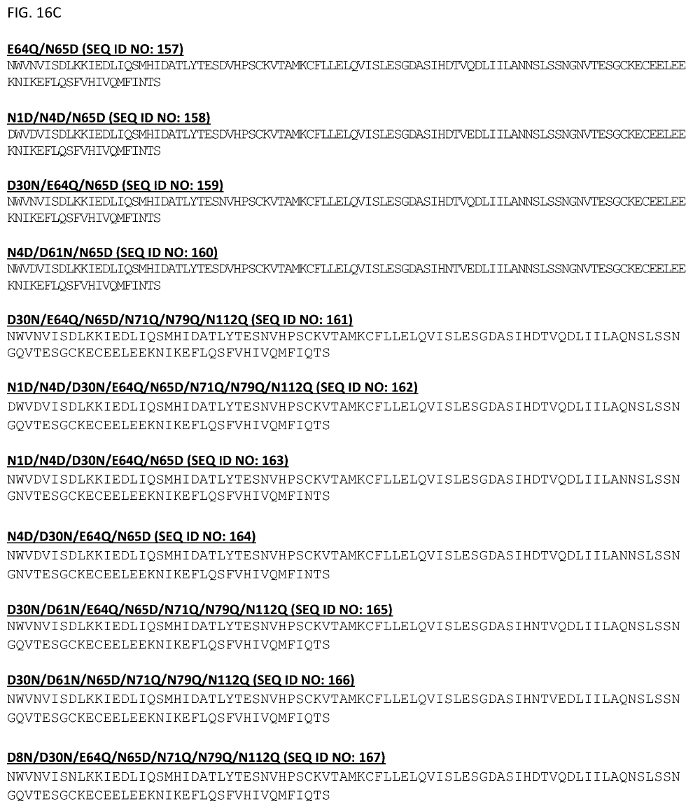

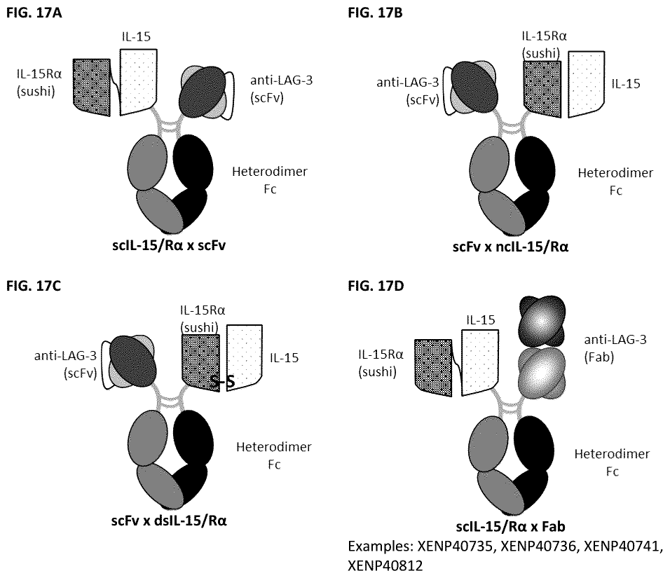

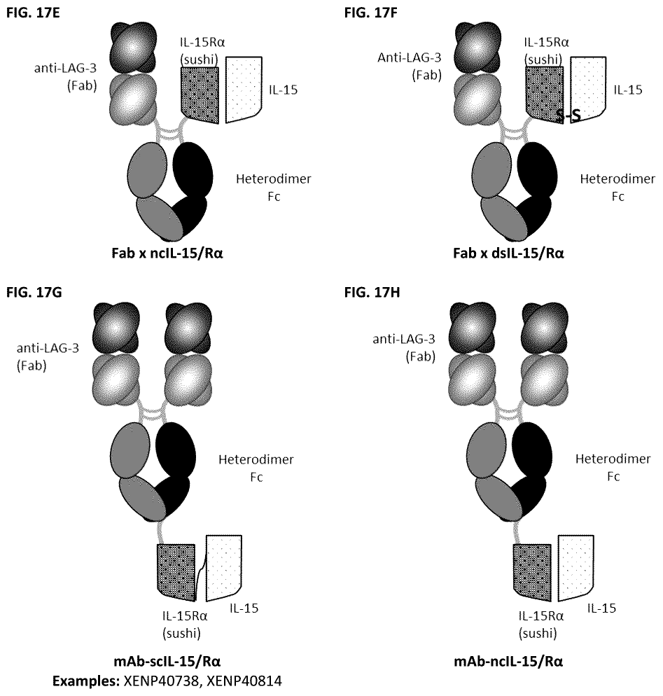

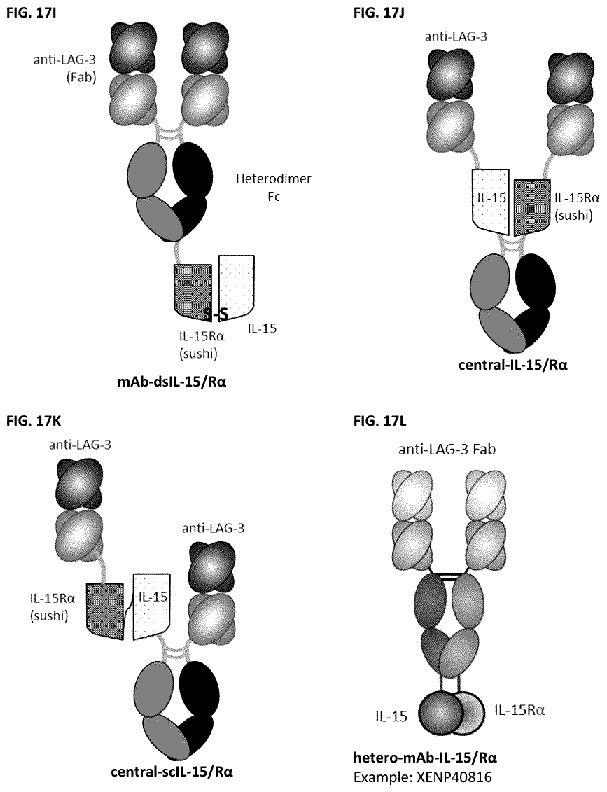

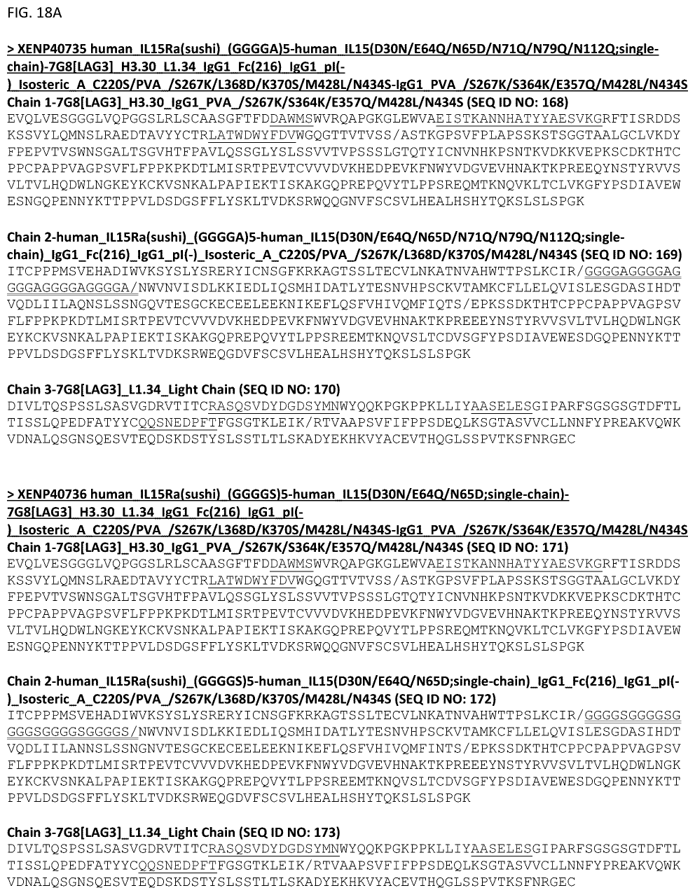

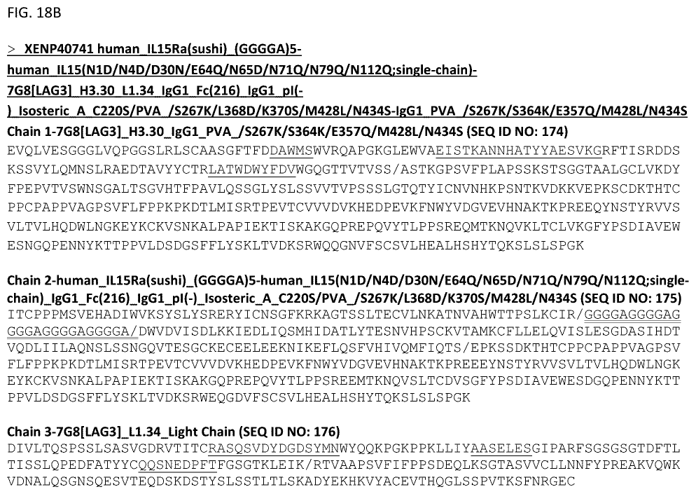

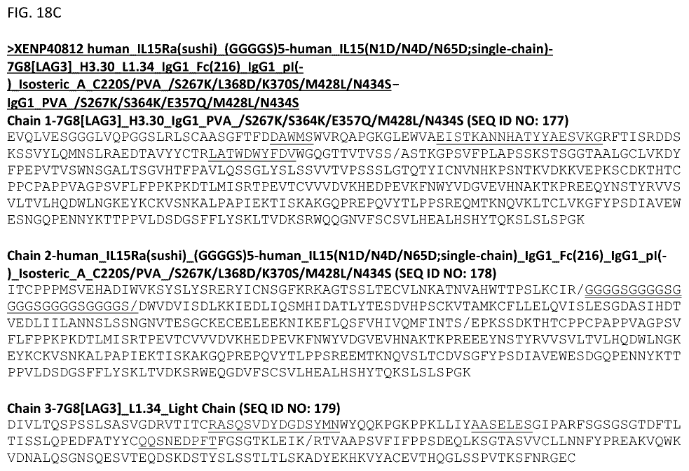

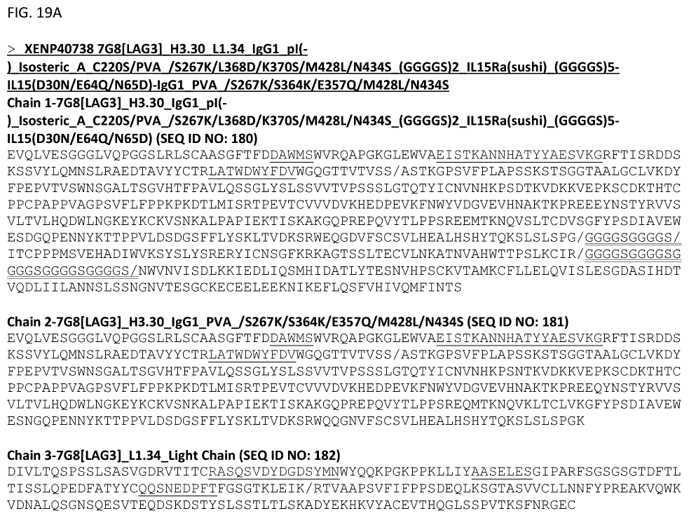

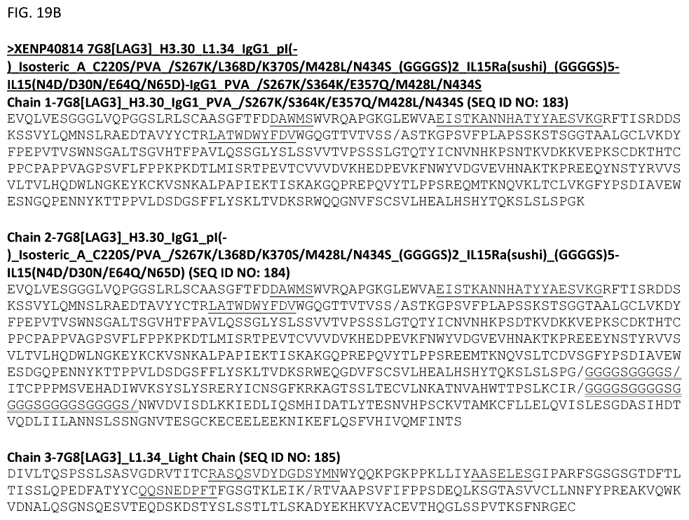

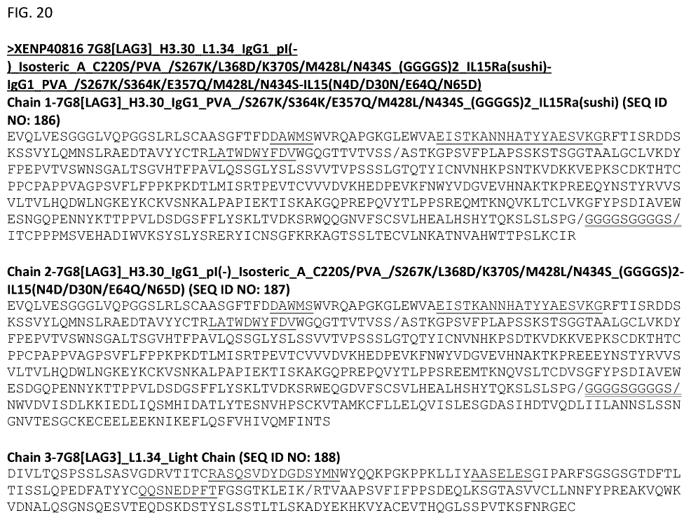

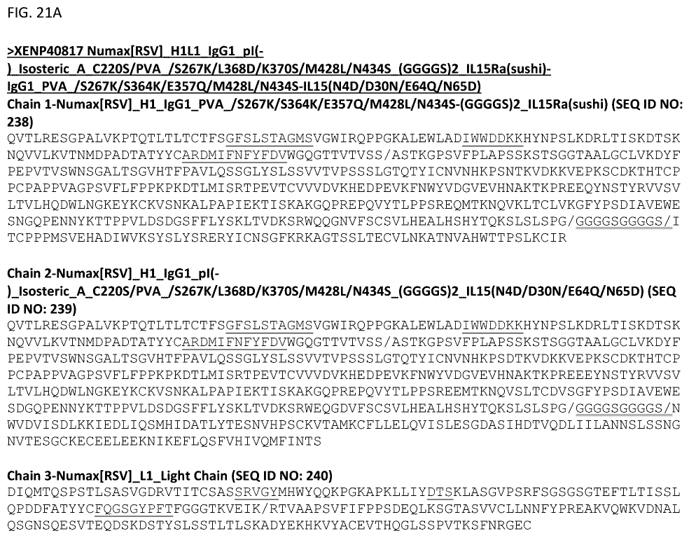

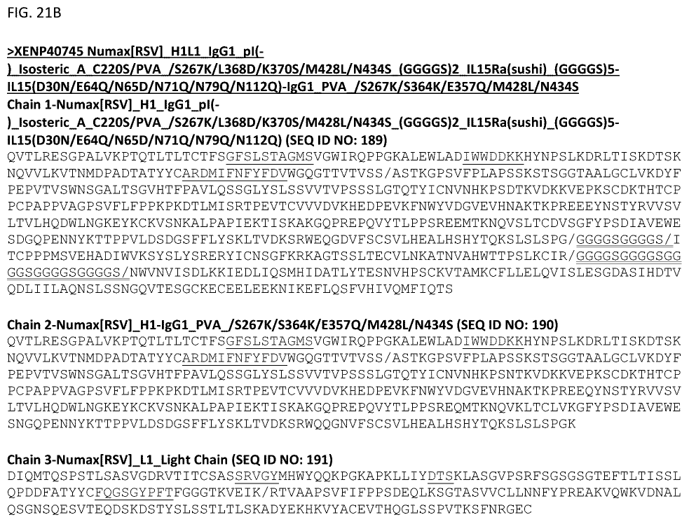

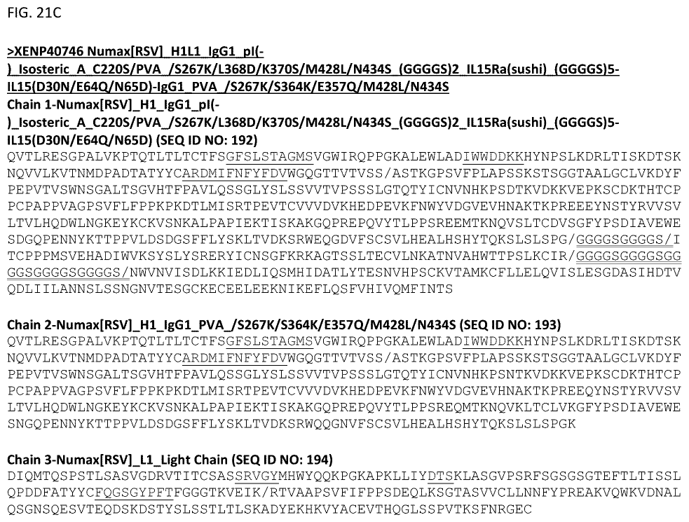

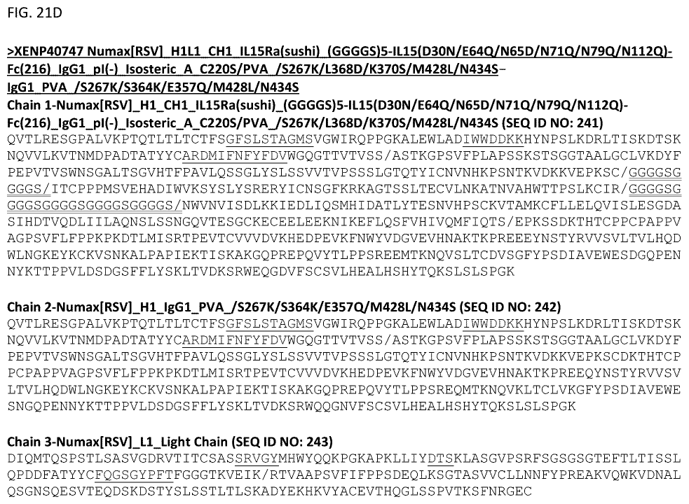

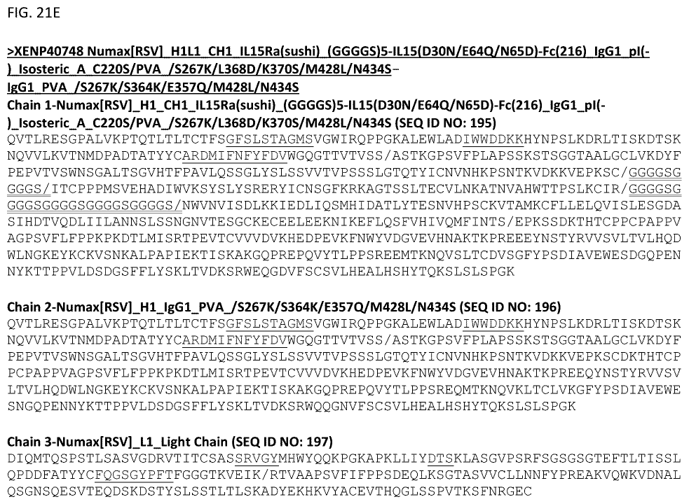

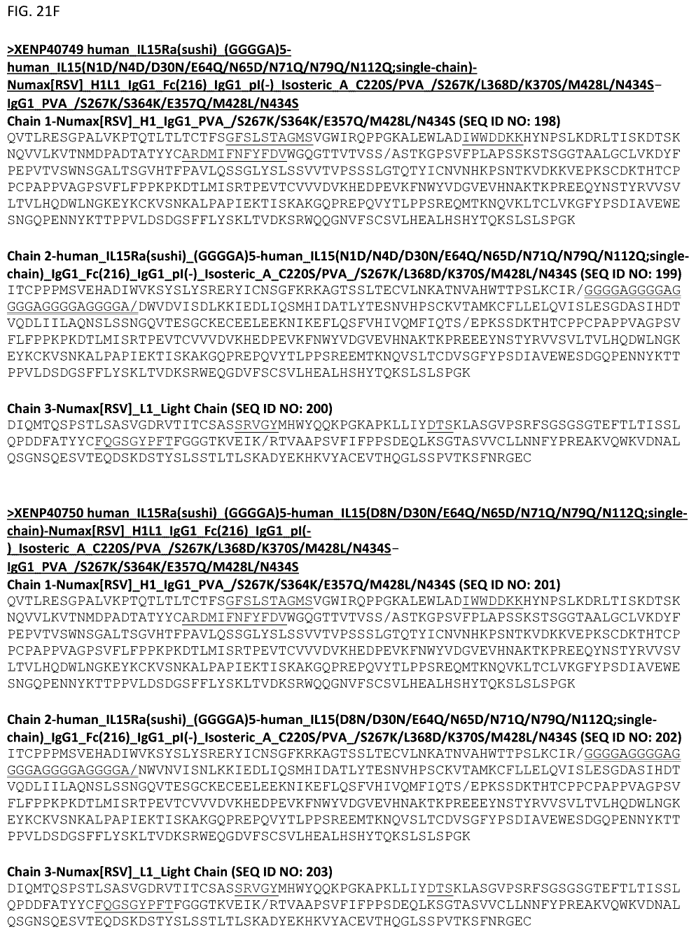

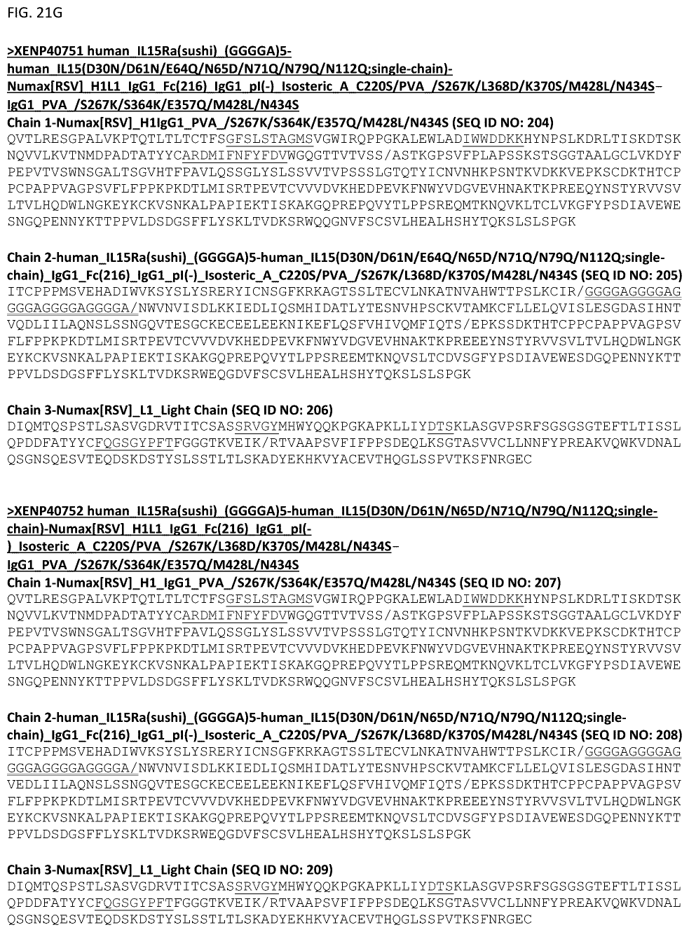

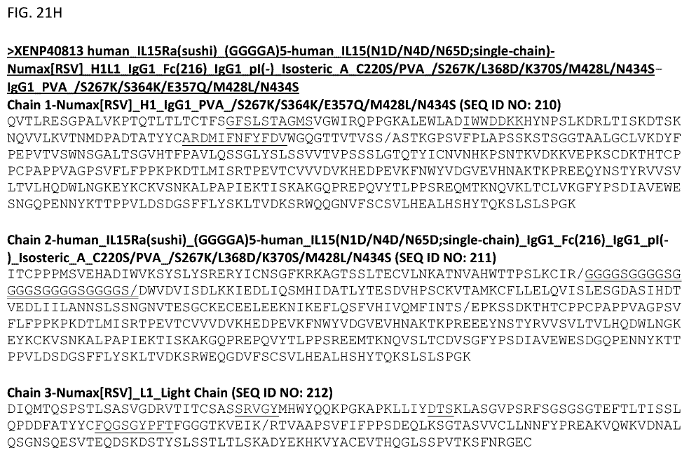

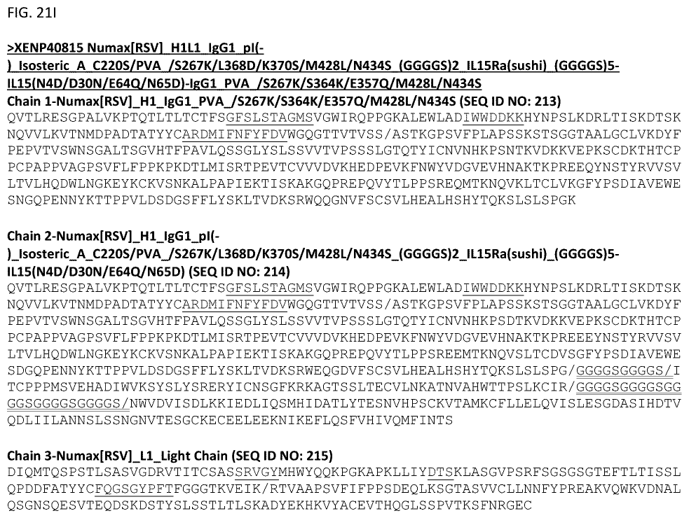

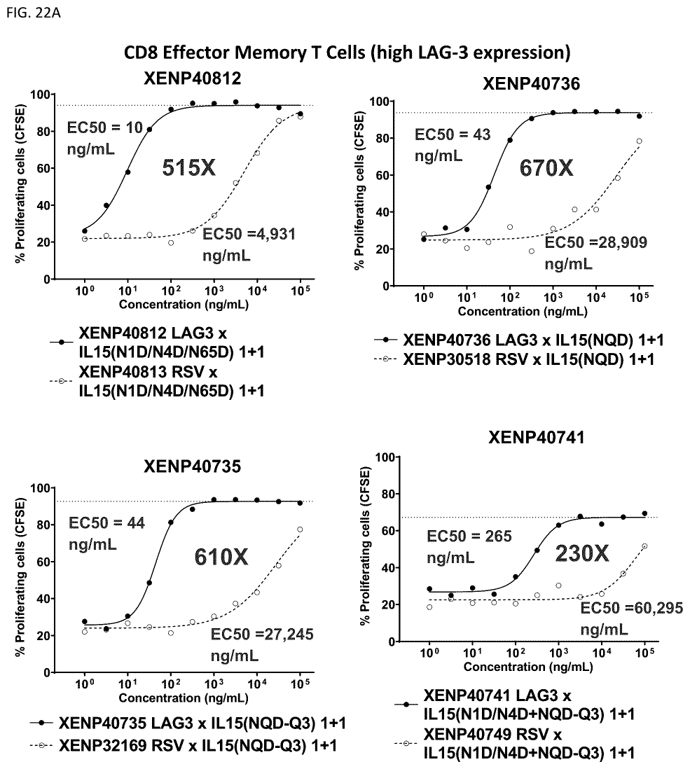

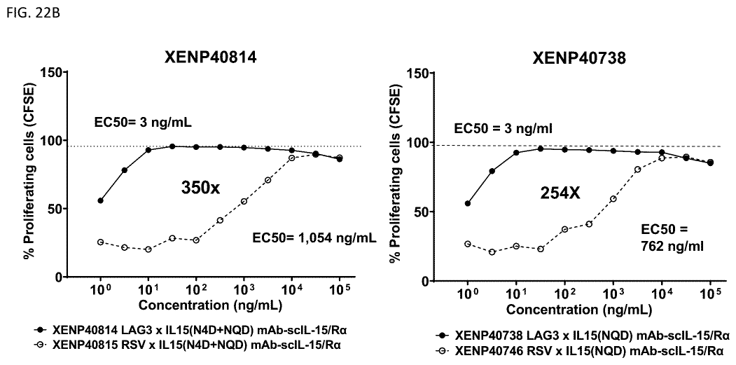

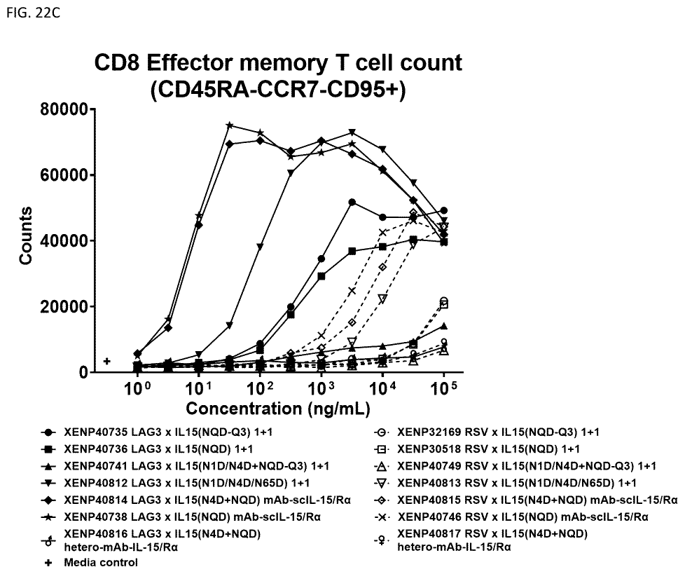

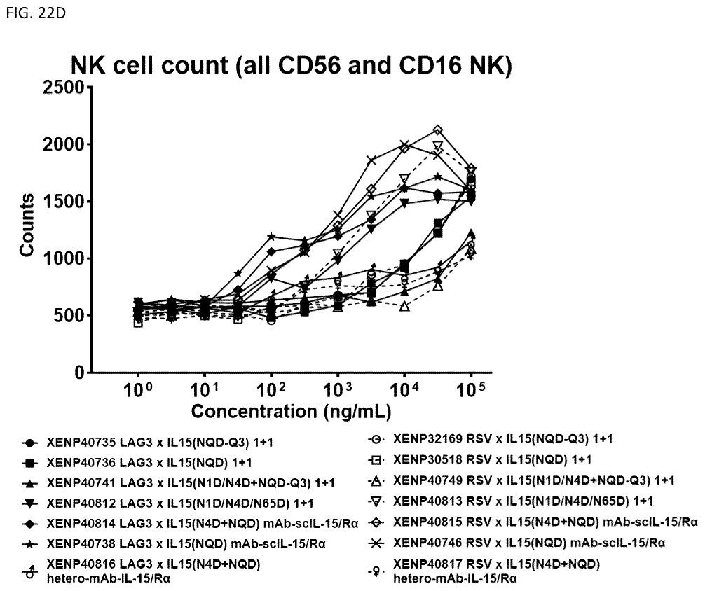

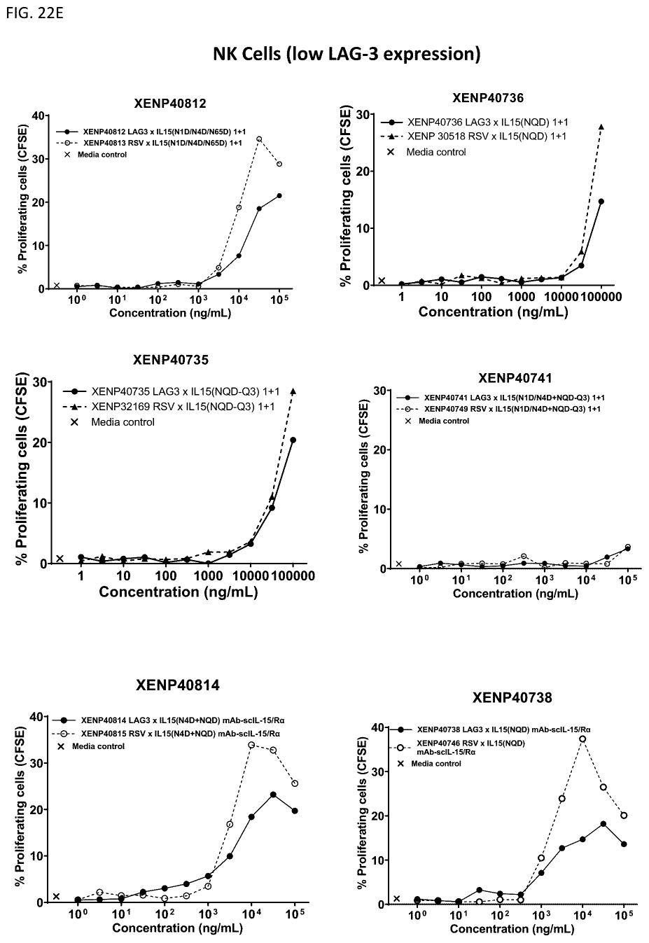

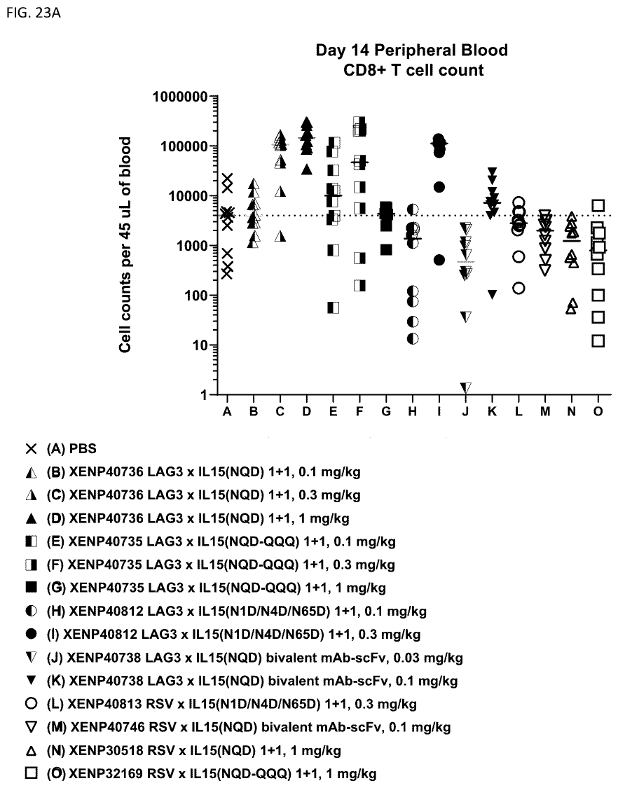

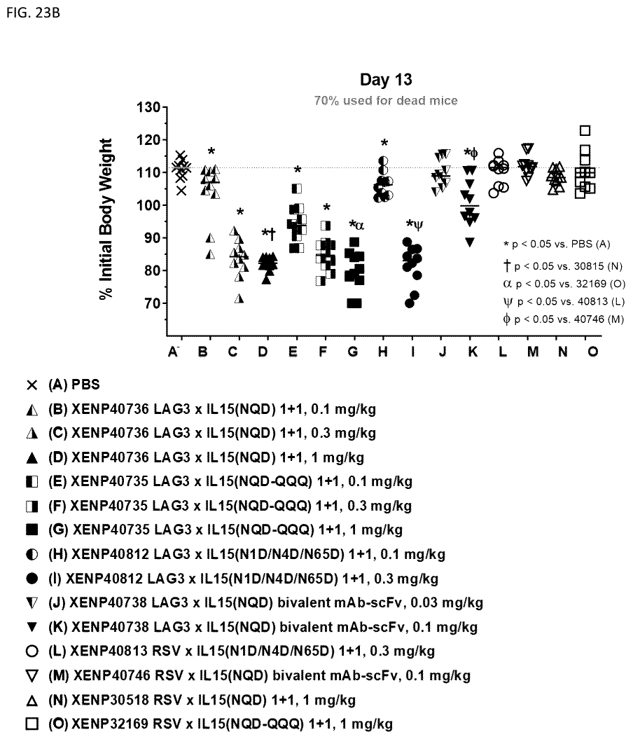

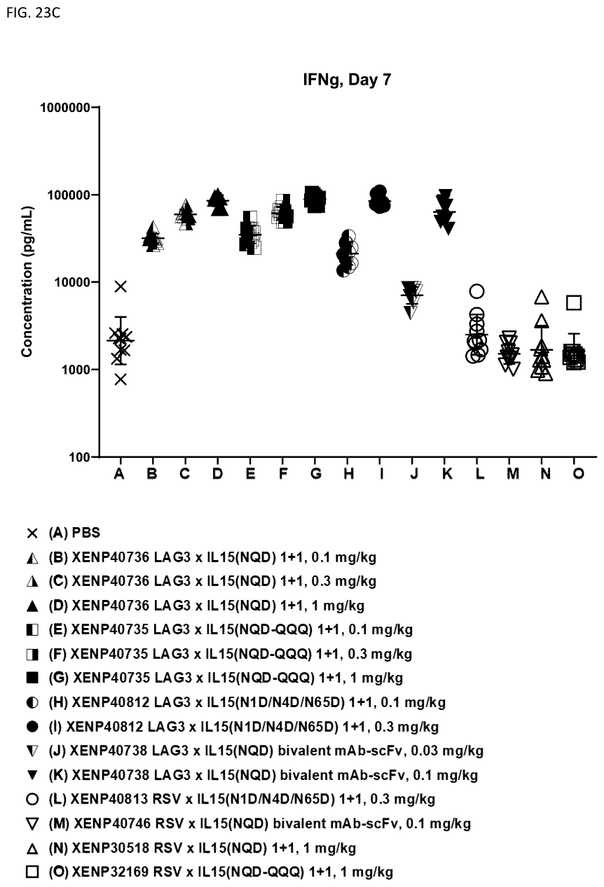

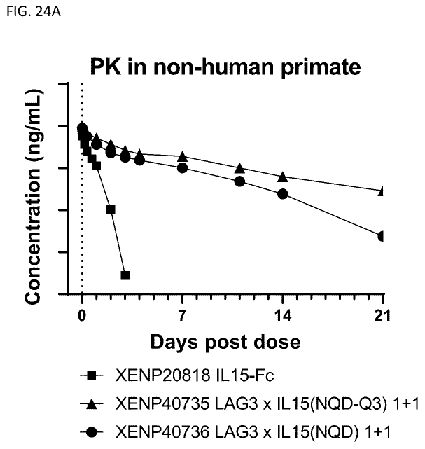

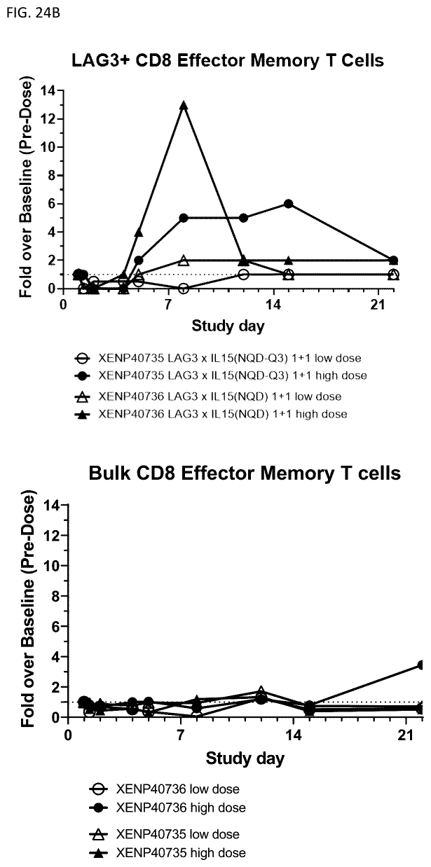

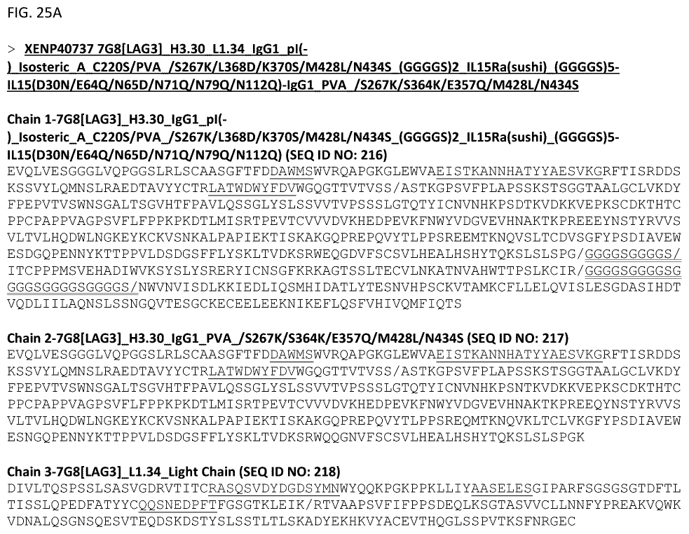

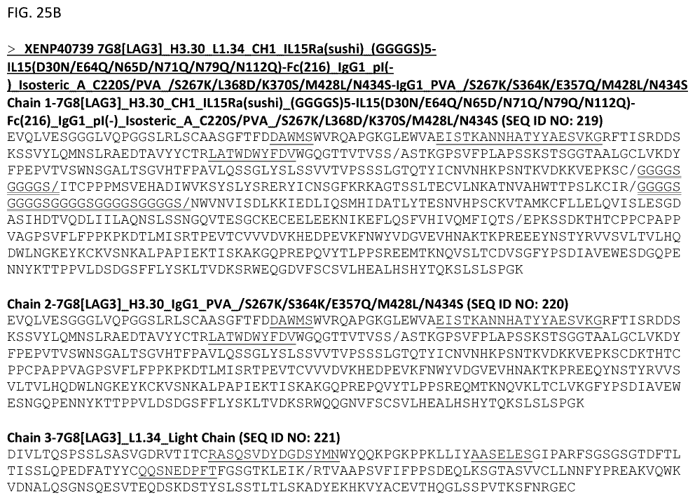

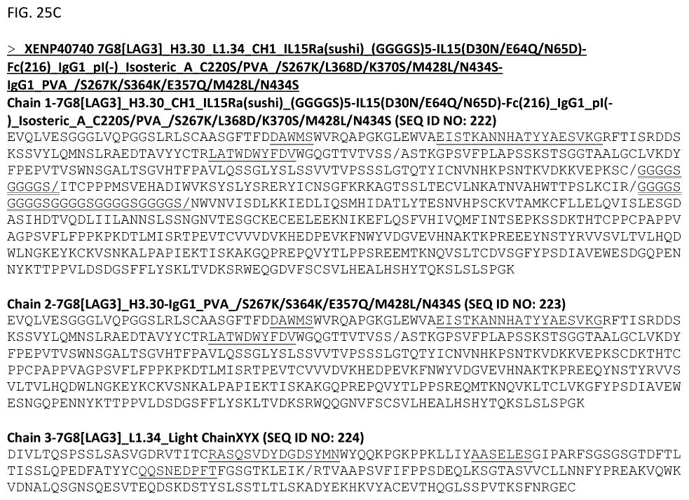

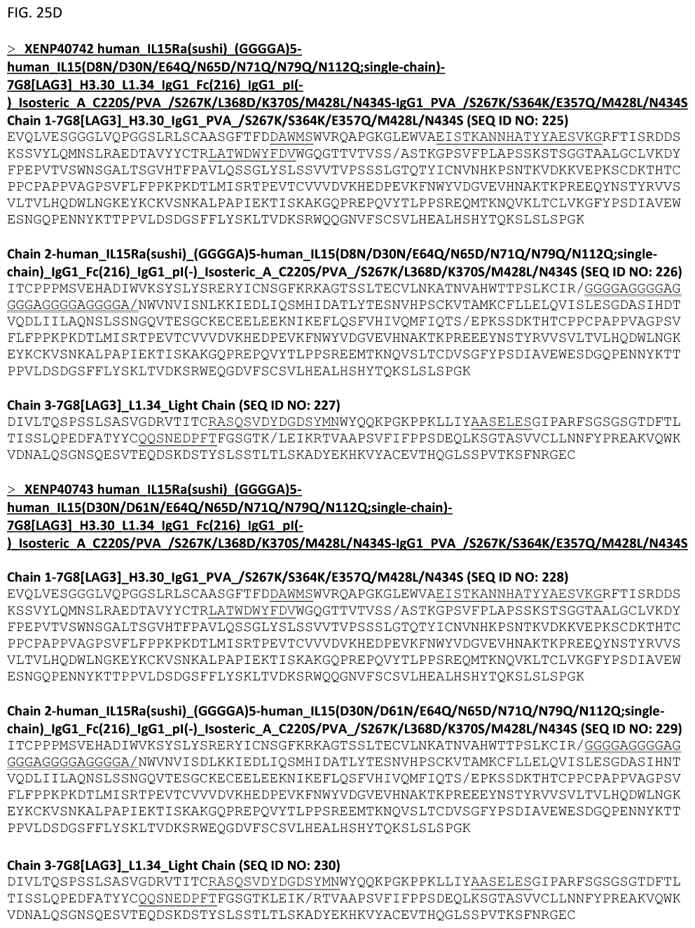

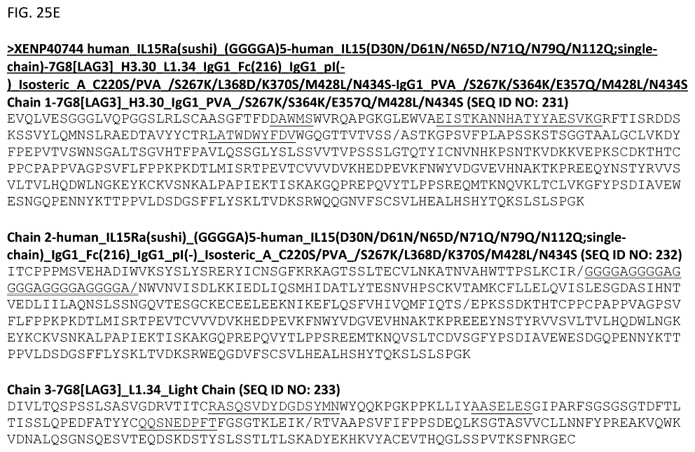

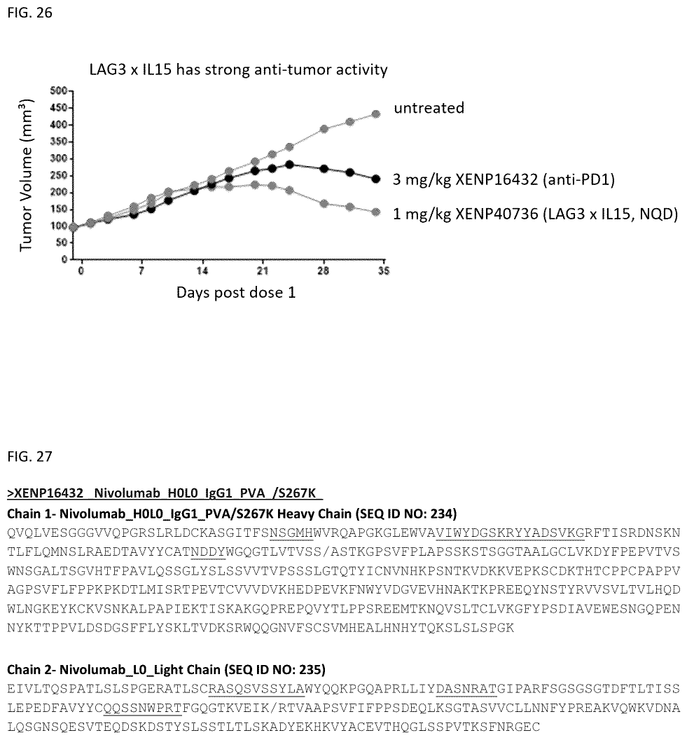

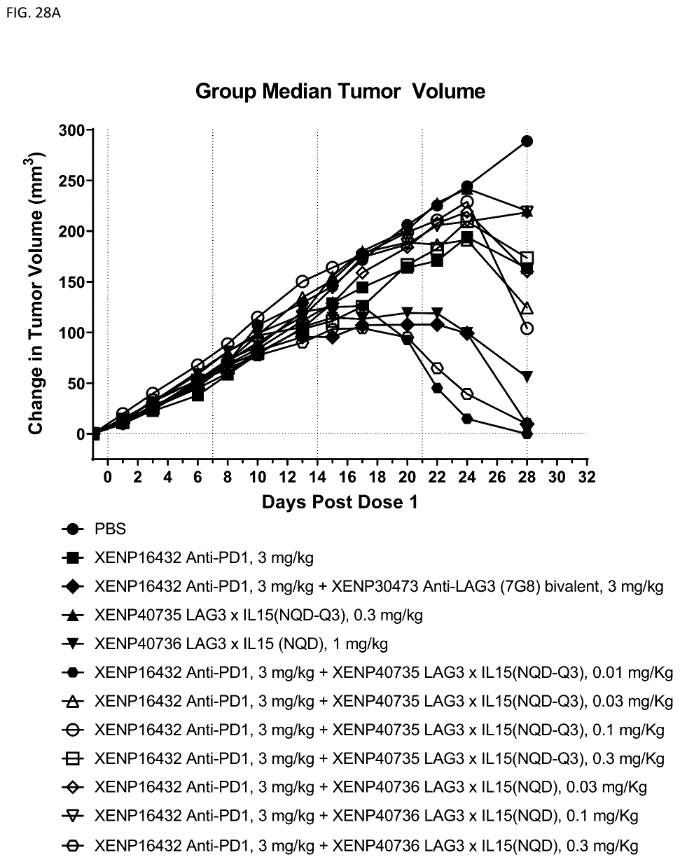

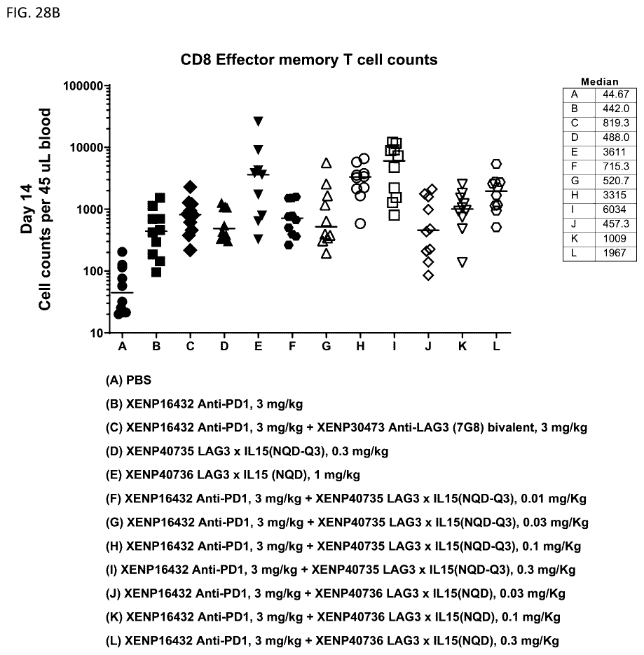

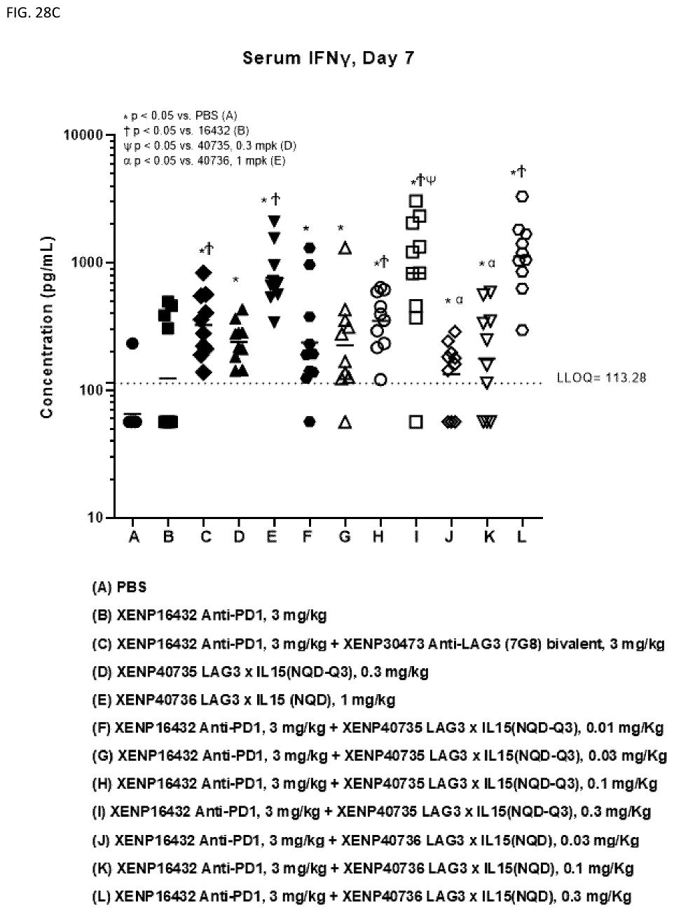

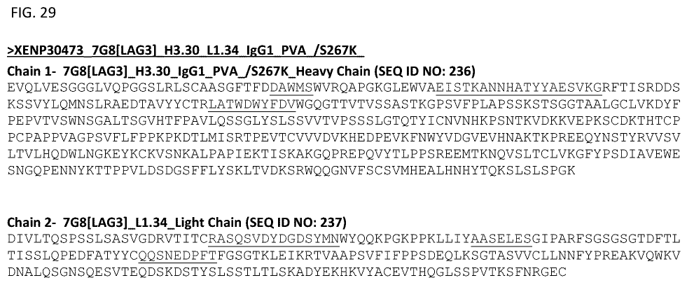

Useful “backbone” sequences that can be included in the “scIL-15/Rα×Fab” format heterodimeric protein are depicted in FIG. 10. In some embodiments, the “scIL-15/Rα×Fab” format heterodimeric protein that includes: a) a first monomer that includes, from N- to C-terminus, an IL-15Rα (sushi) domain-(domain linker)-IL-15 variant-(hinge)-CH2-CH3, where hinge-CH2-CH3 has the amino acid sequence of Chain 2 of “Backbone 1” in FIG. 10 (SEQ ID NO: 68); b) a second monomer that includes, from N- to C-terminus, a VH-CH1-hinge-CH2-CH3, where VH is a variable heavy domain and CH1-hinge-CH2-CH3 has the amino acid sequence of Chain 1 of “Backbone 1” in FIG. 10 (SEQ ID NO: 67), and c) a light chain that includes from, N- to C-terminus, VL-VC, where VL is a variable light domain and VC has the sequence of “Constant Light Chain-Kappa” in FIG. 11 (SEQ ID NO: 73). In certain embodiments, the “scIL-15/Rα×Fab” format heterodimeric protein that includes: a) a first monomer that includes, from N- to C-terminus, an IL-15Rα (sushi) domain-(domain linker)-IL-15 variant-(hinge)-CH2-CH3, where hinge-CH2-CH3 has the amino acid sequence of Chain 2 of “Backbone 2” in FIG. 10 (SEQ ID NO: 70); b) a second monomer that includes, from N- to C-terminus, a VH-CH1-hinge-CH2-CH3, where VH is a variable heavy domain and CH1-hinge-CH2-CH3 has the amino acid sequence of Chain 1 of “Backbone 2” in FIG. 10 (SEQ ID NO: 69), and c) a light chain that includes from, N- to C-terminus, VL-VC, where VL is a variable light domain and VC has the sequence of “Constant Light Chain-Kappa” in FIG. 11 (SEQ ID NO: 73). In some embodiments, the “scIL-15/Rα×Fab” format heterodimeric protein that includes: a) a first monomer that includes, from N- to C-terminus, an IL-15Rα (sushi) domain-(domain linker)-IL-15 variant-(hinge)-CH2-CH3, where hinge-CH2-CH3 has the amino acid sequence of Chain 2 of “Backbone 3” in FIG. 10 (SEQ ID NO: 72); b) a second monomer that includes, from N- to C-terminus, a VH-CH1-hinge-CH2-CH3, where VH is a variable heavy domain and CH1-hinge-CH2-CH3 has the amino acid sequence of Chain 1 of “Backbone 3” in FIG. 10 (SEQ ID NO: 71), and c) a light chain that includes from, N- to C-terminus, VL-VC, where VL is a variable light domain and VC has the sequence of “Constant Light Chain-Kappa” in FIG. 11 (SEQ ID NO: 73). In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the IL-15 variant includes amino acid substitutions N4D/N65D, D30N/N65D, D30N/E64Q/N65D, D30N/E64Q/N65D/N71Q/N79Q/N112Q, N1D/N4D/D30N/E64Q/N65D/N71Q/N79Q/N112Q, N1D/N4D/D30N/E64Q/N65D, N4D/D30N/E64Q/N65D, D30N/D61N/E64Q/N65D/N71Q/N79Q/N112Q, D30N/D61N/N65D/N71Q/N79Q/N112Q, or D8N/D30N/E64Q/N65D/N71Q/N79Q/N112Q. In an exemplary embodiment, the VH and VL are the VH and VL of any of the LAG-3 ABDs in FIGS. 12 and 13A-C and the IL-15 variant includes amino acid substitutions N4D/N65D. In another exemplary embodiment, the VH and VL are the VH and VL of any of the LAG-3 ABDs in FIGS. 12 and 13A-C and the IL-15 variant includes amino acid substitutions D30N/N65D. In yet another exemplary embodiment, the VH and VL are the VH and VL of any of the LAG-3 ABDs in FIGS. 12 and 13A-C and the IL-15 variant includes amino acid substitutions D30N/E64Q/N65D.

Particular preferred LAG-3 targeted IL-15/IL-15Rα-Fc heterodimeric fusion proteins include XENP40735, XENP40736, XENP40741, and XENP40812 “scIL-15/Rα×Fab” format heterodimeric protein, XENP40738 and XENP40814 “mAb-scIL-15/Rα” format heterodimeric protein, and XENP40816 “hetero-mAb-IL-15/Rα” format heterodimeric protein. Exemplary embodiments of the LAG-3 targeted IL-15/IL-15Rα-Fc heterodimeric fusion proteins are shown in FIGS. 18A, B and C, FIGS. 19A and B, and FIG. 20.

### VI. Nucleic Acids of the Invention

In another aspect, provided herein are nucleic acid compositions encoding the targeted heterodimeric fusion proteins (or, in the case of a monomer Fc domain protein, nucleic acids encoding those as well).

As will be appreciated by those in the art, the nucleic acid compositions will depend on the format of the targeted heterodimeric fusion protein. Thus, for example, when the format requires three amino acid sequences, three nucleic acid sequences can be incorporated into one or more expression vectors for expression. Similarly, some formats only two nucleic acids are needed; again, they can be put into one or two expression vectors, or four or 5. As noted herein, some constructs have two copies of a light chain, for example.

As is known in the art, the nucleic acids encoding the components of the invention can be incorporated into expression vectors as is known in the art, and depending on the host cells used to produce the targeted heterodimeric fusion proteins of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors provided herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer, as applicable depending on the format, are each contained within a single expression vector, generally under different or the same promoter controls. In embodiments of particular use in the present invention, each of these two or three nucleic acids are contained on a different expression vector.

The targeted heterodimeric fusion protein of the invention are made by culturing host cells comprising the expression vector(s) as is well known in the art. Once produced, traditional fusion protein or antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the heterodimer (e.g., anionic exchange columns, cationic exchange columns). These substitutions also aid in the determination and monitoring of any contaminating homodimers post-purification (e.g., IEF gels, cIEF, and analytical IEX columns).

### VII. Biological and Biochemical Functionality of Targeted LAG-3 Antibody×IL-15/IL-15Rα Heterodimeric Immunomodulatory Fusion Proteins

Generally the targeted heterodimeric fusion proteins of the invention are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status along with “old fashioned” measurements such as tumor burden, size, invasiveness, LN involvement, metastasis, etc. can be done. Thus, any or all of the following can be evaluated: the inhibitory effects of the heterodimeric proteins on CD4⁺ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of the heterodimeric protein on Treg cell differentiation and proliferation and Treg- or myeloid derived suppressor cell (MDSC)-mediated immunosuppression or immune tolerance, and/or the effects of heterodimeric protein on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and ³H-thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or T6 T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

A. Assays to Measure Efficacy

In some embodiments, T cell activation is assessed using a Mixed Lymphocyte Reaction (MLR) assay as is known in the art. An increase in activity indicates immunostimulatory activity. Appropriate increases in activity are outlined below.

In one embodiment, the signaling pathway assay measures increases or decreases in immune response as measured for an example by phosphorylation or de-phosphorylation of different factors, or by measuring other post translational modifications. An increase in activity indicates immunostimulatory activity. Appropriate increases in activity are outlined below.

In one embodiment, the signaling pathway assay measures increases or decreases in activation of αβ and/or γδ T cells as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in cytotoxic T cell activity as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in NK and/or NKT cell activity as measured for an example by direct killing of target cells like for an example cancer cells or by cytokine secretion or by changes in expression of activation markers like for an example CD107a, etc. An increase in activity indicates immunostimulatory activity. Appropriate increases in activity are outlined below.

In one embodiment, the signaling pathway assay measures increases or decreases in αβ and/or γδ T-cell suppression, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in pro-inflammatory cytokine secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in Th1 response as measured for an example by cytokine secretion or by changes in expression of activation markers. An increase in activity indicates immunostimulatory activity. Appropriate increases in activity are outlined below.

In one embodiment, the signaling pathway assay measures increases or decreases in Th2 response as measured for an example by cytokine secretion or by changes in expression of activation markers. An increase in activity indicates immunostimulatory activity. Appropriate increases in activity are outlined below.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophage pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined below.

In one embodiment, the signaling pathway assay measures increases or decreases in N2 neutrophils pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in inhibition of T cell activation, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in inhibition of CTL activation as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in αβ and/or γδ T cell exhaustion as measured for an example by changes in expression of activation markers. A decrease in response indicates immunostimulatory activity. Appropriate decreases are the same as for increases, outlined below.

In one embodiment, the signaling pathway assay measures increases or decreases αβ and/or γδ T cell response as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in stimulation of antigen-specific memory responses as measured for an example by cytokine secretion or by proliferation or by changes in expression of activation markers like for an example CD45RA, CCR7 etc. An increase in activity indicates immunostimulatory activity. Appropriate increases in activity are outlined below.

In one embodiment, the signaling pathway assay measures increases or decreases in apoptosis or lysis of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases in stimulation of cytotoxic or cytostatic effect on cancer cells. As measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases direct killing of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases Th17 activity as measured for an example by cytokine secretion or by proliferation or by changes in expression of activation markers. An increase in activity indicates immunostimulatory activity. Appropriate increases in activity are outlined below.

In one embodiment, the signaling pathway assay measures increases or decreases in induction of complement dependent cytotoxicity and/or antibody dependent cell-mediated cytotoxicity, as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, T cell activation is measured for an example by direct killing of target cells like for an example cancer cells or by cytokine secretion or by proliferation or by changes in expression of activation markers like for an example CD137, CD107a, PD1, etc. For T-cells, increases in proliferation, cell surface markers of activation (e.g., CD25, CD69, CD137, PD1), cytotoxicity (ability to kill target cells), and cytokine production (e.g., IL-2, IL-4, IL-6, IFNγ, TNF-α, IL-10, IL-17A) would be indicative of immune modulation that would be consistent with enhanced killing of cancer cells.

In one embodiment, NK cell activation is measured for example by direct killing of target cells like for an example cancer cells or by cytokine secretion or by changes in expression of activation markers like for an example CD107a, etc. For NK cells, increases in proliferation, cytotoxicity (ability to kill target cells and increases CD107a, granzyme, and perforin expression), cytokine production (e.g., IFNγ and TNF), and cell surface receptor expression (e.g., CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of 10%, 20%, 30%, 40%, 50%, 60%, 70%, 80%, 90%, 95% or 98 to 99% percent over the signal in either a reference sample or in control samples, for example test samples that do not contain a heterodimeric protein of the invention. Similarly, increases of at least one-, two-, three-, four- or five-fold as compared to reference or control samples show efficacy.

### VIII. Treatments

Once made, the compositions of the invention find use in a number of oncology applications, by treating cancer, generally by promoting T cell activation (e.g., T cells are no longer suppressed) with the binding of the heterodimeric fusion proteins of the invention.

Accordingly, the targeted heterodimeric compositions of the invention find use in the treatment of these cancers.

A. Targeted Heterodimeric Protein Compositions for In Vivo Administration

Formulations of the antibodies used in accordance with the present invention are prepared for storage by mixing an antibody having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions. Acceptable carriers, buffers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

B. Combination Therapies

In some embodiments, the heterodimeric proteins of the invention can be used in combination therapies with antibodies that bind to different checkpoint proteins, e.g., not LAG-3 antibodies. In this way, the antigen binding domains of the additional antibody do not compete for binding with the targeted heterodimeric protein. In this way, a sort of “triple combination” therapy is achieved, as three receptors are engaged (two from the targeted heterodimeric protein and one from the additional antibody). As discussed herein, the heterodimeric protein can have different valencies and specifities as outlined herein.

Surprisingly, as shown herein, these combinations can result in synergistic effects when co-administered. In this context, “co-administration” means that the two moieties can be administered simultaneously or sequentially. That is, in some cases, the drugs may be administered simultaneously, although generally this is through the use of two separate IV infusions; that is, the drugs are generally not combined into a single dosage unit. Alternatively, co-administration includes the sequential administration of the two separate drugs, either in a single day or separate days (including separate days over time).

1. Anti-PD-1 Antibodies for Use in Co-Administration Therapies

As is known in the art, there are two currently approved anti-PD-1 antibodies and many more in clinical testing. Thus, suitable anti-PD-1 antibodies for use in combination therapies as outlined herein include, but are not limited to, the two currently FDA approved antibodies, pembrolizumab and nivolizumab, as well as those in clinical testing currently, including, but not limited to, tislelizumab, Sym021, REGN2810 (developed by Rengeneron), JNJ-63723283 (developed by J and J), SHR-1210, pidilizumab, AMP-224, MEDIo680, PDR001 and CT-001, as well as others outlined in Liu et al., J. Hemat. & Oncol. (2017)10:136, the antibodies therein expressly incorporated by reference. As above, anti-PD-1 antibodies are used in combination when the targeted heterodimeric proteins of the invention do not have an antigen binding domain that binds PD-1.

2. Anti-PD-L1 Antibodies for Use in Co-Administration Therapies

In some embodiments, anti-PD-L1 antibodies are used in combination. As is known in the art, there are three currently approved anti-PD-L1 antibodies and many more in clinical testing. Thus, suitable anti-PD-L1 antibodies for use in combination therapies as outlined herein include, but are not limited to, the three currently FDA approved antibodies, atezolizumab, avelumab, durvalumab, as well as those in clinical testing currently, including, but not limited to, LY33000054 and CS1001, as well as others outlined in Liu et al., J. Hemat. & Oncol. (2017)10:136, the antibodies therein expressly incorporated by reference. As above, anti-PD-L1 antibodies are used in combination when the targeted heterodimeric proteins of the invention do not have an antigen binding domain that binds PD-L1.

3. Anti-TIM-3 Antibodies for Use in Co-Administration Therapies

In some embodiments, anti-TIM-3 antibodies can be used in combination with the targeted heterodimeric proteins of the invention. There are several TIM-3 antibodies in clinical development, including MBG453 and TSR-022. As above, anti-TIM-3 antibodies are used in combination when the targeted heterodimeric proteins of the invention do not have an antigen binding domain that binds TIM-3.

4. Anti-TIGIT Antibodies for Use in Co-Administration Therapies

In some embodiments, anti-TIGIT antibodies can be used in combination with the targeted heterodimeric proteins of the invention. There are several TIGIT antibodies in clinical development, BMS-986207, OMP-313M32 and MTIG7192A. As above, anti-TIGIT antibodies are used in combination when the targeted heterodimeric proteins of the invention do not have an antigen binding domain that binds TIGIT.

5. Anti-CTLA-4 Antibodies for Use in Co-Administration Therapies

In some embodiments, anti-CTLA-4 antibodies can be used in combination with the targeted heterodimeric proteins of the invention. Ipilimumab has been approved, and there are several more in development, including CP-675,206 and AGEN-1884. As above, anti-CTLA-4 antibodies are used in combination when the targeted heterodimeric proteins of the invention do not have an antigen binding domain that binds CTLA-4.

C. Administrative Modalities

The targeted heterodimeric proteins and chemotherapeutic agents of the invention are administered to a subject, in accord with known methods, such as intravenous administration as a bolus or by continuous infusion over a period of time.

D. Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the targeted heterodimeric protein used in the present invention depend on the disease or condition to be treated and may be determined by the persons skilled in the art.

An exemplary, non-limiting range for a therapeutically effective amount of a targeted heterodimeric protein used in the present invention is about 0.1-100 mg/kg.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### IX. Examples

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Patent Publications 2015/0307629, 2014/0288275 and WO2014/145806, all of which are expressly incorporated by reference in their entirety and particularly for the techniques outlined therein. Examples 1 and 2 from U.S. Ser. No. 62,416,087, filed on Nov. 1, 2016 are expressly incorporated by reference in their entirety, including the corresponding figures. Additionally, U.S. Ser. Nos. 62/408,655, 62/443,465, 62/477,926, 15/785,401, 62/416,087 and 15/785,393 are expressly incorporated by reference in their entirety, and specifically for all the sequences, Figures and Legends therein.

### A. Example 1: Anti-LAG3 Binding Domains

Examples of anti-LAG3 Fv domain were previously described in WO2017/218707, the contents of which are hereby incorporated by reference in their entirety. The variable heavy and variable light domain sequences for an exemplary anti-LAG3 binding domain of the invention can be seen in FIG. 14. Human and predicted cynomolgus LAG-3 sequences are depicted in FIG. 3.

### B. Example 2: LAG3 Targeted IL-15/IL-15Rα-Fc Fusions

Reference is made to U.S. Patent Pub. No. US 2019/0352362 A1, hereby expressly incorporated by reference in its entirety, which previously described numerous formats for LAG3×IL15/IL-15Rα-Fc fusion proteins and their generation.

Plasmids coding for IL-15, IL-15Rα sushi domain, or the anti-LAG-3 variable regions were constructed by standard gene synthesis, followed by subcloning into a pTT5 expression vector containing Fc fusion partners (e.g., constant regions as depicted in FIG. 10). Cartoon schematics of illustrative LAG-3-targeted IL-15/Rα-Fc fusion formats are depicted in FIG. 17. Several formats have found particular use in the present invention.

Firstly, the “scIL-15/Rα×Fab” format (FIG. 17D) comprises an IL-15Rα (sushi) fused to IL-15 by a variable length linker (termed “scIL-15/Rα”) which is then fused to the N-terminus of a heterodimeric Fc-region, with a variable heavy chain (VH) fused to the other side of the heterodimeric Fc, while a corresponding light chain is transfected separately so as to form a Fab with the VH. XENP40735, XENP40736, XENP40741, and XENP40812 are examples of this scIL-15/Rα×Fab format, and their sequences are illustrated in FIG. 18.

Secondly, the “mAb-scIL-15/Rα” format (FIG. 17G) comprises VH fused to the N-terminus of a first and a second heterodimeric Fc, with IL-15 is fused to IL-15Rα (sushi) which is then further fused to the C-terminus of one of the heterodimeric Fc-region, while corresponding light chains are transfected separately so as to form a Fabs with the VHs. XENP40738 and XENP40814, sequences for which are depicted in FIG. 19, are examples of the mAb-scIL-15/Rα format.

Thirdly, the “hetero-mAb-IL-15/Rα” format (FIG. 17L) comprises VH fused to the N-terminus of a first and a second heterodimeric Fc, where IL-15 is fused to the C-terminus of one heterodimeric Fc-region, and IL-15Rα (sushi) is fused to the C-terminus of the other heterodimeric Fc-region, and corresponding light chains are transfected separately so as to form a Fabs with the VHs. XENP40816 is an example of the bivalent mAb-Fv-IL-15Rα format, and its sequence is illustrated in FIG. 20.

### C. Example 3: IL-15 Variants

While the LAG-3-targeted IL-15/Rα-Fc fusions were designed with the aim to be targeted to the tumor environment via the LAG-3-targeting arm, the cytokine moiety is still capable of signaling before reaching the tumor site, which may contribute to systemic toxicity. Therefore, potency-reduced IL15/IL15Rα were created by engineering amino acid substitutions in IL15—at the IL2Rβ/γc interface, which were able to significantly reduce in vitro potency. The sequences for IL-15 variants used in the invention are illustrated in FIG. 16. As one example, XENP40816 (FIG. 20) comprises IL-15 potency variants N4D/D30N/E64Q/N65D. Additionally, as glycosylation may lead to faster clearance and poor PK in vivo, some IL-15 potency reduction variants are combined with aglycosylation variants. For example, XENP40735 comprises potency reduction variants D30N/E64Q/N65D (which may also be referred to as “NQD” throughout) combined with aglycosylation variants N71Q/N79Q/N112Q (which may also be referred to as “QQQ” throughout). As another example, XENP40741 (FIG. 18) comprises IL-15 potency variants N1D/N4D/D30N/E64Q/N65D combined with aglycosylation variants N71Q/N79Q/N112Q.

### D. Example 4: LAG-3×IL-15 Demonstrates Potent Activity and has High Selectivity for LAG3+ Cells

In order to test the activity of LAG-3×IL-15 constructs in vitro, an experiment was performed in which human PBMCs were activated with 500 ng/ml plate-bound anti-CD3 (OKT3) for 48 hours. Cells were labeled with carboxyfluorescein succinimidyl ester (CFSE). Next, either LAG-3×IL-15 constructs or RSV×IL-15 constructs were added to the cells at a range of concentrations. RSV×IL-15 constructs each correspond to a particular LAG-3×IL-15 construct, having the same IL-15 variant and the same format, but having an anti-RSV binding domain instead of a LAG-3 binding domain, in order to act as a control for untargeted activity. Sequences for RSV×IL-15 test articles are depicted in FIG. 21. After 4 days at 37° C., the cells were analyzed by flow cytometry in order to measure their proliferation by the concentration of CFSE. As depicted in FIG. 22, on CD8 EM T cells, which have high LAG-3 expression, most LAG3×IL-15 constructs showed significantly higher (>500 fold) induction of proliferation than did their corresponding RSV×IL-15 counterparts, as measured by their EC50 values. Additionally, LAG-3×IL-15 constructs were ˜3-fold more potent on CD8T cells compared to CD4 T cells (data not shown). On NK cells, which have minimal LAG-3 expression, the LAG-3×TL-15 constructs induced very little proliferation, and no more than the corresponding RSV×IL-15 control. These results demonstrated that LAG-3×IL-15 constructs have high selectivity for LAG3+ cell phenotypes such as CD8-EM T cells, and very low activity on cells with low LAG-3 expression, such as NK cells.

### E. Example 5: LAG3×IL15 Promotes T Cell Proliferation and IFNγ Production in GVHD Mouse Model

In order to further investigate the LAG-3×IL-15 constructs in vivo, an experiment was conducted utilizing a Graft-versus-Host-Disease (GvHD) mouse model, in which NSG (NOD-SCID-gamma) immunodeficient mice are engrafted with human PBMCs, which then develop an autoimmune response against mouse cells. In this study, 10 million human PBMCs were engrafted into NSG mice via IV-OSP on Day 0, followed by intraperitoneal injections of the test articles at the indicated concentrations on Days 0, 7, and 14. Weekly bleeds for cytokine analysis occurred on Days 7 and 14. Peripheral blood samples were stained with an antibody cocktail against surface antigens and analyzed by flow cytometry to enumerate cell subsets. Cell count values of 0 were set to 1 for median calculations, analyses, and plotting. Statistical significance of cell counts for group comparisons were determined using an unpaired Mann-Whitney nonparametric test using Prism, and IFN□ levels were measured. Clinical observations of GvHD and body weight measurements were taken twice a week, the data for which is shown in FIG. 23B. FIG. 23 depicts the results of the study, demonstrating that LAG3×IL15 constructs can successfully promote CD8+ T cell proliferation (FIG. 23A) and IFNγ production (FIG. 23C) in vivo. In particular, XENP40736 showed a strong, dose-dependent increase in CD8+ T cell counts at Day 14. Both XENP40735 (D30N/E64Q/N65D IL-15) and XENPN40736 (D30N/E64Q/N65D/N71Q/N79Q/N112Q IL-15) showed a strong dose-dependent increase in IFNγ. While the LAG3×IL15 constructs resulted in this immune response, the RSV×IL-15 control test articles did not produce an increase in CD8+ T cells compared to the mice injected with PBS buffer, nor did they produce an increase in IFNγ production nor a change in body weight relative to mice in the PBS group. Taken together these results suggest that the LAG3 targeted potency-reduced IL-15-Fc constructs of the invention display a promising profile of selective IL-15 delivery to tumor infiltrating lymphocytes while avoiding unwanted activity in the periphery.

### F. Example 6: LAG3×I15 Demonstrates Favorable In Vivo Properties in Non Human Primates

In a study investigating the pharmacokinetics of LAG3×IL-15-Fc variants, cynomolgus monkeys were administered a first single intravenous (IV) dose of XENP40735, XENP40736, or wild-type IL-15-Fc. The results depicted in FIG. 24A demonstrate that both XENP40735 and XENP40736 have a significantly superior PK as compared to wild-type IL-15-Fc and were generally well-tolerated. Additionally, as demonstrated in FIG. 24B, LAG3×IL15 shows selectivity for LAG3+ cells as compared to CD8 effector memory T cells in general.

### G. Example 7: LAG3×I15 Demonstrates Potent Anti-Tumor Activity In Vivo

Female CD34+ huNSG mice were each inoculated with 3×10⁶ pp65-MCF7-gfp cells on Day −15. On Day 0, mice were treated with a range of doses of either XENP40735 or XENP40736, each either as a single agent or in combination with anti-PD1 XENP16432, as indicated in the legend of FIG. 28. Treatments were delivered in 0.1 ml volume intraperitonially, and were repeated once a week for 5 weeks. Tumor volume was measured by caliper 1-3× per week, and body weight measurements and blood draws were performed weekly. Peripheral blood samples were stained with an antibody cocktail against surface and intracellular antigens and analyzed by flow cytometry to enumerate cell subsets. Cell count values of 0 were set to 1 for analyses and plotting. As depicted in FIG. 26, XENP40736 displayed strong anti-tumor activity as a single agent. Furthermore, as depicted in FIG. 28A, both XENP40736 and XENP 40735 demonstrated anti-tumor activity, as well as proliferation of CD8+ T cells (FIG. 28B), and IFNγ production (FIG. 28C) in this CD34+ humanized mouse model.

1-23. (canceled)

24. A LAG-3-targeted IL-15/Rα-Fc fusion protein comprising:
a) a first monomer comprising the amino acid sequence set forth in SEQ ID NO: 168;
b) a second monomer comprising the amino acid sequence set forth in SEQ ID NO:169; and
c) a third monomer comprising the amino acid sequence set forth in SEQ ID NO: 170.

25. A nucleic acid composition comprising:
a) a first nucleic acid encoding said first monomer of claim 24;
b) a second nucleic acid encoding said second monomer of claim 24; and
c) a third nucleic acid encoding said third monomer of claim 24.

26. An expression vector composition comprising:
a) a first expression vector comprising said first nucleic acid of claim 25;
b) a second expression vector comprising said second nucleic acid of claim 25; and
c) a third expression vector comprising said third nucleic acid of claim 25.

27. A host cell comprising said expression vector composition of claim 26.

28. A method of making a LAG-3-targeted IL-15/Rα-Fc fusion protein according to claim 24 comprising culturing said host cell of claim 27 under conditions wherein said LAG-3-targeted IL-15/Rα-Fc fusion protein of claim 24 is expressed, and recovering said composition.


SEQUENCE LISTING (245 sequences)

SEQ ID NO: 1 - protein, 162 aa, Homo sapiens
  source 1..162: note = sp|P40933
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
  source 1..114: note = sp|P40933|49-162
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
  source 1..267: note = sp|Q13261
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 65 aa, Homo sapiens
  source 1..65: note = sp|Q13261|31-95
ITCPPPMSVEHADIWVKSYSLYSRERYICNSGFKRKAGTSSLTECVLNKATNVAHWTTPSLKCIR

SEQ ID NO: 5 - protein, 175 aa, Homo sapiens
  source 1..175: note = sp|Q13261|31-205
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 6 - protein, 551 aa, Homo sapiens
  source 1..551: note = sp|P14784
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 7 - protein, 214 aa, Homo sapiens
  source 1..214: note = sp|P14784|27-240
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 8 - protein, 369 aa, Homo sapiens
  source 1..369: note = sp|P31785
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 240 aa, Homo sapiens
  source 1..240: note = sp|P31785|23-262
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 10 - protein, 525 aa, Homo sapiens
  source 1..525: note = sp|P18627
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 11 - protein, 422 aa, Homo sapiens
  source 1..422: note = sp|P18627|29-450
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 12 - protein, 533 aa, Macaca fascicularis
  source 1..533: note = gi|544467815|ref|XP_005570011.1
MWEAQFLGLLFLQPLWVAPVKPPQPGAEISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 13 - protein, 422 aa, Macaca fascicularis
  source 1..422: note = gi|544467815|ref|XP_005570011.1|29-450
ISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHL

SEQ ID NO: 14 - protein, 5 aa, synthetic construct
GGGGS

SEQ ID NO: 15 - protein, 10 aa, synthetic construct
GGGGSGGGGS

SEQ ID NO: 16 - protein, 15 aa, synthetic construct
GGGGSGGGGSGGGGS

SEQ ID NO: 17 - protein, 20 aa, synthetic construct
GGGGSGGGGSGGGGSGGGGS

SEQ ID NO: 18 - protein, 25 aa, synthetic construct
GGGGSGGGGSGGGGSGGGGSGGGGS

SEQ ID NO: 19 - protein, 30 aa, synthetic construct
GGGGSGGGGSGGGGSGGGGSGGGGSGGGGS

SEQ ID NO: 20 - protein, 35 aa, synthetic construct
GGGGSGGGGSGGGGSGGGGSGGGGSGGGGSGGGGS

SEQ ID NO: 21 - protein, 5 aa, synthetic construct
GGGGA

SEQ ID NO: 22 - protein, 10 aa, synthetic construct
GGGGAGGGGA

SEQ ID NO: 23 - protein, 15 aa, synthetic construct
GGGGAGGGGAGGGGA

SEQ ID NO: 24 - protein, 20 aa, synthetic construct
GGGGAGGGGAGGGGAGGGGA

SEQ ID NO: 25 - protein, 25 aa, synthetic construct
GGGGAGGGGAGGGGAGGGGAGGGGA

SEQ ID NO: 26 - protein, 30 aa, synthetic construct
GGGGAGGGGAGGGGAGGGGAGGGGAGGGGA

SEQ ID NO: 27 - protein, 35 aa, synthetic construct
GGGGAGGGGAGGGGAGGGGAGGGGAGGGGAGGGGA

SEQ ID NO: 28 - protein, 5 aa, synthetic construct
AGGGG

SEQ ID NO: 29 - protein, 5 aa, synthetic construct
EAAAK

SEQ ID NO: 30 - protein, 30 aa, synthetic construct
DPALVHQRPAPPGGGGSGGGGSGGGGSGGG

SEQ ID NO: 31 - protein, 5 aa, synthetic construct
GKPGS

SEQ ID NO: 32 - protein, 25 aa, synthetic construct
GKPGSGKPGSGKPGSGKPGSGKPGS

SEQ ID NO: 33 - protein, 30 aa, synthetic construct
GKPGSGKPGSGKPGSGKPGSGKPGSGKPGS

SEQ ID NO: 34 - protein, 5 aa, synthetic construct
GGGES

SEQ ID NO: 35 - protein, 15 aa, synthetic construct
EPKSCDKTHTCPPCP

SEQ ID NO: 36 - protein, 9 aa, synthetic construct
KTHTCPPCP

SEQ ID NO: 37 - protein, 15 aa, synthetic construct
EPKSSDKTHTCPPCP

SEQ ID NO: 38 - protein, 19 aa, synthetic construct
GGGGSGGGGSKTHTCPPCP

SEQ ID NO: 39 - protein, 19 aa, synthetic construct
GKPGSGKPGSKTHTCPPCP

SEQ ID NO: 40 - protein, 14 aa, synthetic construct
GKPGSKTHTCPPCP

SEQ ID NO: 41 - protein, 18 aa, synthetic construct
GSTSGSGKPGSGEGSTKG

SEQ ID NO: 42 - protein, 14 aa, synthetic construct
IRPRAIGGSKPRVA

SEQ ID NO: 43 - protein, 15 aa, synthetic construct
GKGGSGKGGSGKGGS

SEQ ID NO: 44 - protein, 15 aa, synthetic construct
GGKGSGGKGSGGKGS

SEQ ID NO: 45 - protein, 15 aa, synthetic construct
GGGKSGGGKSGGGKS

SEQ ID NO: 46 - protein, 15 aa, synthetic construct
GKGKSGKGKSGKGKS

SEQ ID NO: 47 - protein, 15 aa, synthetic construct
GGGKSGGKGSGKGGS

SEQ ID NO: 48 - protein, 15 aa, synthetic construct
GKPGSGKPGSGKPGS

SEQ ID NO: 49 - protein, 20 aa, synthetic construct
GKPGSGKPGSGKPGSGKPGS

SEQ ID NO: 50 - protein, 20 aa, synthetic construct
GKGKSGKGKSGKGKSGKGKS

SEQ ID NO: 51 - protein, 14 aa, synthetic construct
STAGDTHLGGEDFD

SEQ ID NO: 52 - protein, 15 aa, synthetic construct
GEGGSGEGGSGEGGS

SEQ ID NO: 53 - protein, 15 aa, synthetic construct
GGEGSGGEGSGGEGS

SEQ ID NO: 54 - protein, 15 aa, synthetic construct
GGGESGGGESGGGES

SEQ ID NO: 55 - protein, 15 aa, synthetic construct
GEGESGEGESGEGES

SEQ ID NO: 56 - protein, 15 aa, synthetic construct
GGGESGGEGSGEGGS

SEQ ID NO: 57 - protein, 20 aa, synthetic construct
GEGESGEGESGEGESGEGES

SEQ ID NO: 58 - protein, 19 aa, synthetic construct
PRGASKSGSASQTGSAPGS

SEQ ID NO: 59 - protein, 19 aa, synthetic construct
GTAAAGAGAAGGAAAGAAG

SEQ ID NO: 60 - protein, 19 aa, synthetic construct
GTSGSSGSGSGGSGSGGGG

SEQ ID NO: 61 - protein, 10 aa, synthetic construct
EAAAKEAAAK

SEQ ID NO: 62 - protein, 20 aa, synthetic construct
EAAAKEPKSSDKTHTCPPCP

SEQ ID NO: 63 - protein, 5 aa, synthetic construct
GSGGS

SEQ ID NO: 64 - protein, 4 aa, synthetic construct
GGGS

SEQ ID NO: 65 - protein, 5 aa, synthetic construct
GAGGA

SEQ ID NO: 66 - protein, 4 aa, synthetic construct
GGGA

SEQ ID NO: 67 - protein, 329 aa, synthetic construct
  source 1..329: note = IL-15/Ralpha x anti-LAG-3 Backbone 1 Chain 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 231 aa, synthetic construct
  source 1..231: note = IL-15/Ralpha x anti-LAG-3 Backbone 1 Chain 2
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 69 - protein, 329 aa, synthetic construct
  source 1..329: note = IL-15/Ralpha x anti-LAG-3 Backbone 2 Chain 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 70 - protein, 231 aa, synthetic construct
  source 1..231: note = IL-15/Ralpha x anti-LAG-3 Backbone 2 Chain 2
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 329 aa, synthetic construct; identical to SEQ ID NO: 69
  source 1..329: note = IL-15/Ralpha x anti-LAG-3 Backbone 3 Chain 1

SEQ ID NO: 72 - protein, 329 aa, synthetic construct
  source 1..329: note = IL-15/Ralpha x anti-LAG-3 Backbone 3 Chain 2
ASTKGPSVFPLAPSSKSTSGGTAALGCLVKDYFPEPVTVSWNSGALTSGVHTFPAVLQSSGLYSLSSVVTVPSSSLGTKTYTCNVNHKPSNTKVDKKVERKSCDKTHTCPRCPAPPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 107 aa, synthetic construct [sequence omitted]

SEQ ID NO: 74 - protein, 106 aa, synthetic construct
  source 1..106: note = Constant Light Chain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 121 aa, synthetic construct
  source 1..121: note = 7G8[LAG-3]_H0L0 Variable Heavy
EVKLEESGGGLVQPGGSMKLSCAASGFTFSDAWMDWVRQSPEKGLEWVAEIRTKANNHATYYAESVKGRFTISRDDSKSSVYLQMNSLRAEDTGIYYCTRLANWDWYFDVWGAGTTVTVSS

SEQ ID NO: 76 - protein, 111 aa, synthetic construct
  source 1..111: note = 7G8[LAG-3]_H0L0 Variable Light
DTVLTQSPASLAVSLGQRATISCKASQSVDYDGDSYMNWYQQKPGQPPKLLIYAASNLESGIPARLSGSGSGTDFTLNIHPVEEEDAATYYCQQSNEDPFTFGSGTKLEVK

SEQ ID NO: 77 - protein, 121 aa, synthetic construct
  source 1..121: note = 7G8[LAG-3]_H3L1 Variable Heavy
EVQLVESGGGLVQPGGSLRLSCAASGFTFSDAWMDWVRQAPGKGLEWVAEIRTKANNHATYYAESVKGRFTISRDDSKSSVYLQMNSLRAEDTAVYYCTRLANWDWYFDVWGQGTTVTVSS

SEQ ID NO: 78 - protein, 111 aa, synthetic construct
  source 1..111: note = 7G8[LAG-3]_H3L1 Variable Light
DTVLTQSPSSLSASVGDRVTITCRASQSVDYDGDSYMNWYQQKPGKPPKLLIYAASNLESGIPARLSGSGSGTDFTLTISSLQPEDFATYYCQQSNEDPFTFGSGTKLEIK

SEQ ID NO: 79 - protein, 121 aa, synthetic construct
  source 1..121: note = 7G8[LAG-3]_H3.30_L1.34 Variable Heavy
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSS

SEQ ID NO: 80 - protein, 111 aa, synthetic construct
  source 1..111: note = 7G8[LAG-3]_H3.30_L1.34 Variable Light
DIVLTQSPSSLSASVGDRVTITCRASQSVDYDGDSYMNWYQQKPGKPPKLLIYAASELESGIPARFSGSGSGTDFTLTISSLQPEDFATYYCQQSNEDPFTFGSGTKLEIK

SEQ ID NO: 81 - protein, 118 aa, synthetic construct
  source 1..118: note = 2A11[LAG-3]_H0L0 Variable Heavy
EVQLQQSGAELVRSGASVKLSCTASGFNIKDYYMHWVKQRPEQGLEWIGWIDPENGDTEYAPKFQGKATMTADTSSNTAYLQLSSLTSEDTAVYYCYARGVRQALDYWGQGTSVTVSS

SEQ ID NO: 82 - protein, 107 aa, synthetic construct
  source 1..107: note = 2A11[LAG-3]_H0L0 Variable Light
DIQMTQTTSSLSASLGDRVTISCRASQDIGNYLNWFQQKPDGTVKLLIYYTSRLHSGVPSRFSGSGSGTDYSLTISNLEQEDIATYFCQQGNTLPYTFGGGTKLEIK

SEQ ID NO: 83 - protein, 118 aa, synthetic construct
  source 1..118: note = 2A11[LAG-3]_H1L2 Variable Heavy
EVQLVQSGAEVKKPGATVKISCKASGFNIKDYYMHWVQQAPGKGLEWMGWIDPENGDTEYAPKFQGRVTITADTSTNTAYMELSSLRSEDTAVYYCYARGVRQALDYWGQGTLVTVSS

SEQ ID NO: 84 - protein, 107 aa, synthetic construct
  source 1..107: note = 2A11[LAG-3]_H1L2 Variable Light
DIQMTQSPAFLSVTPGEKVTITCQASQDIGNYLNWFQQKPDQTVKLLIYYTSRLHSGVPSRFSGSGSGTDYTFTISSLEAEDAATYFCQQGNTLPYTFGGGTKVEIK

SEQ ID NO: 85 - protein, 118 aa, synthetic construct
  source 1..118: note = 2A11[LAG-3]_H1.144_L2.142 Variable Heavy
EVQLVQSGAEVKKPGATVKISCKASGFNIKDYFMHWVQQAPGKGLEWMGWIDPELGDTEYAPKFQGRVTITADTSTNTAYMELSSLRSEDTAVYYCYARGVYQALDYWGQGTLVTVSS

SEQ ID NO: 86 - protein, 107 aa, synthetic construct
  source 1..107: note = 2A11[LAG-3]_H1.144_L2.142 Variable Light
DIQMTQSPAFLSVTPGEKVTITCQASQDIGNYLNWFQQKPGQTVKLLIYFTSYLHSGVPSRFSGSGSGTDYTFTISSLEAEDAATYFCQQGNTLPYTFGGGTKVEIK

SEQ ID NO: 87 - protein, 120 aa, synthetic construct
  source 1..120: note = BMS-986016[LAG-3] Variable Heavy
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 88 - protein, 107 aa, synthetic construct
  source 1..107: note = BMS-986016[LAG-3] Variable Light
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 89 - protein, 116 aa, synthetic construct
  source 1..116: note = IMP731[LAG-3] Variable Heavy
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSS

SEQ ID NO: 90 - protein, 114 aa, synthetic construct
  source 1..114: note = IMP731[LAG-3] Variable Light
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKR

SEQ ID NO: 91 - protein, 123 aa, synthetic construct
  source 1..123: note = 13E2[LAG-3] Variable Heavy
QVTLKESGPGILQPSQTLSLTCSFSGFSLSTSGMGLGWIRQPSGKGLEWLTHIWWDDIKRYNPDLRSRLTISKDTSSSQIFLKIASVDTADTATYYCARIVEGSYSSSYFDVWGAGTTVTVSS

SEQ ID NO: 92 - protein, 107 aa, synthetic construct
  source 1..107: note = 13E2[LAG-3] Variable Light
DIVMTQPHKFMSTSVEDRVTITCKASQDVIFDVAWYQQKPGQSPKLLIYSASSRVSGVPDRFTGSGSGTDFTFTISSVQAEDLAVYYCQQHYSTPYTFGGGTTLEIK

SEQ ID NO: 93 - protein, 123 aa, synthetic construct
  source 1..123: note = 34F4[LAG-3] Variable Heavy
QVTLKESGPGILQPSQTLSLTCSFSGFSLNTSGMGVGWIRQPSGKGLEWLTHIWWDDVKRYNPALKSRLTISKDTSSSQVFLKIASVDTADTATYYCARIEGDTYYDYYFDYWGQGVTLTVSS

SEQ ID NO: 94 - protein, 107 aa, synthetic construct
  source 1..107: note = 34F4[LAG-3] Variable Light
DIVMTQSHKLMSTSVGDGLSITCKASQDVSIAVVWYQQKPGQSPKLLIYSASFRYTGVPDRFTGSGSGTDFTFTISSVQAEDLAVYYCQQHYSIPWTFGGGTKLEIK

SEQ ID NO: 95 - protein, 123 aa, synthetic construct
  source 1..123: note = IMP761[LAG-3] Variable Heavy
QITLKESGPTLVKPTQTLTLTCTFSGFSLSTSGMGLGWIRQPPGKTLEWLTHIWWDDIKRYNPDLRSRLSITKDTSKNQVVLTMTNMDPLDTGTYYCARIVEGSYSSSYFDVWGQGTLVTVSS

SEQ ID NO: 96 - protein, 107 aa, synthetic construct
  source 1..107: note = IMP761[LAG-3] Variable Light
DIVMTQTPSSLSASVGDRVTITCKASQDVIFDVAWYQQRPGQAPKLLIYSASSRVSGVPSRFSGSGSGTDFTLTISSLQPEDFATYYCQQHYSTPYTFGQGTRLDIK

SEQ ID NO: 97 - protein, 116 aa, synthetic construct
  source 1..116: note = H5L7[LAG-3] Variable Heavy
QVQLQESGPGLVKPSETLSLTCTVSGFSLTAYGVNWIRQPPGKGLEWIGMIWDDGSTDYDSALKSRVTISVDTSKNQFSLKLSSVTAADTAVYYCAREGDVAFDYWGQGTLVTVSS

SEQ ID NO: 98 - protein, 114 aa, synthetic construct
  source 1..114: note = H5L7[LAG-3] Variable Light
DIQMTQSPSSLSASVGDRVTITCKSSQSLLNPSNQKNYLAWYQQKPGKAPKLLVYFASTRDSGVPSRFSGSGSGTDFTLTISSLQPEDFATYYCLQHFGTPPTFGQGTKLEIKR

SEQ ID NO: 99 - protein, 119 aa, synthetic construct
  source 1..119: note = hu22D2[LAG-3] Variable Heavy
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSS

SEQ ID NO: 100 - protein, 111 aa, synthetic construct
  source 1..111: note = hu22D2[LAG-3] Variable Light
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 101 - protein, 123 aa, synthetic construct
  source 1..123: note = H4sH15482P[LAG-3] Variable Heavy
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 102 - protein, 107 aa, synthetic construct
  source 1..107: note = H4sH15482P[LAG-3] Variable Light
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 103 - protein, 119 aa, synthetic construct
  source 1..119: note = L35D4[LAG-3] Variable Heavy
QVQLVQSGAEVKKPGASVKVSCKASGYTFTSYYMHWVRQAPGQGLEWMGIINPSAGSTSYAQKFQGRVTMTRDTSTSTVYMELSSLRSEDTAVYYCARELMATGGFDYWGQGTLVTVSS

SEQ ID NO: 104 - protein, 112 aa, synthetic construct
  source 1..112: note = L35D4[LAG-3] Variable Light
QSVLTQPPSASGSPGQSVTISCTGTSSDVGGYNYVSWYQQYPGKAPRLMIFEVTERASGVPDRFSGSKSGNTASLTVSGLQTEDEAVYFCSSYSGSNNPGAMFGGGTKLTVL

SEQ ID NO: 105 - protein, 119 aa, synthetic construct; identical to SEQ ID NO: 103
  source 1..119: note = L35G6[LAG-3] Variable Heavy

SEQ ID NO: 106 - protein, 110 aa, synthetic construct
  source 1..110: note = L35G6[LAG-3] Variable Light
QAGLTQPASVSGSPGQSITISCTGSSSDVGGYSYVSWYQKHPGKAPKLMIYDVTNRPSGVSNRFSGSKSGNTASLTISGLQAEDEADYYCSTYTRSNTLVFGPGTKVTVL

SEQ ID NO: 107 - protein, 119 aa, synthetic construct; identical to SEQ ID NO: 103
  source 1..119: note = L33H11[LAG-3] Variable Heavy

SEQ ID NO: 108 - protein, 110 aa, synthetic construct
  source 1..110: note = L33H11[LAG-3] Variable Light
LPVLTQPASVSGSPGQSITISCTGTSSDVGGYNYVSWYQQHPGKAPKLMIYDVTNRPSGVSNRFSGSKSGNTASLTISGLQAEDEADYYCSSYTSSNTLLFGGGTQLTVL

SEQ ID NO: 109 - protein, 119 aa, synthetic construct; identical to SEQ ID NO: 103
  source 1..119: note = L32A9[LAG-3] Variable Heavy

SEQ ID NO: 110 - protein, 110 aa, synthetic construct
  source 1..110: note = L32A9[LAG-3] Variable Light
QSVVTQPPSVSAAPGQKVTISCSGSSSNIGNNYVSWYQQLPGTAPKLLIYDNNKRHSGIPDRFSGSTSDTSATLGITRLQTGDEADYYCGTWDSSLSAYVFGTGTKVTVL

SEQ ID NO: 111 - protein, 119 aa, synthetic construct; identical to SEQ ID NO: 103
  source 1..119: note = L32D10[LAG-3] Variable Heavy

SEQ ID NO: 112 - protein, 110 aa, synthetic construct
  source 1..110: note = L32D10[LAG-3] Variable Light
QSVLTQPPSASGSPGQSVTISCTGTSSDVGGYDYVSWYQQHQGKAPKLMIYDVSNRPSGVSNRFSGSKSGNTASLTISGLQAEDEADYYCSSYTSSTTLVFGGGTKLTVL

SEQ ID NO: 113 - protein, 119 aa, synthetic construct; identical to SEQ ID NO: 103
  source 1..119: note = L32A4[LAG-3] Variable Heavy

SEQ ID NO: 114 - protein, 110 aa, synthetic construct
  source 1..110: note = L32A4[LAG-3] Variable Light
QSVLTQPASVSGSPGQSITISCTGTSSDIGAYNFVSWYQQHPGKAPKLMIYGVSNRPSGVSSRFSGSKSGSTASLTISGLQAEDEADYYCSSYTTSGSAVFGTGTKLTVL

SEQ ID NO: 115 - protein, 119 aa, synthetic construct
  source 1..119: note = L3A1[LAG-3] Variable Heavy
EVQLLESGAEVKKPGASVKVSCKASGYTFTSYYMHWVRQAPGQGLEWMGIINPSAGSTSYAQKFQGRVTMTRDTSTSTVYMELSSLRSEDTAVYYCARELMATGGFDYWGQGTLVTVSS

SEQ ID NO: 116 - protein, 110 aa, synthetic construct
  source 1..110: note = L3A1[LAG-3] Variable Light
QSVLTQPASVSGSPGQSITISCTGTSSDIGAYNFVSWYQQHPGKAPKLMIYGVSNRPSGVSSRFSGSKSGSTASLTITGLQAEDEADYYCSSYTTSGSAVFGTGTKLTVL

SEQ ID NO: 117 - protein, 121 aa, synthetic construct
  source 1..121: note = L3A10[LAG-3] Variable Heavy
EVQLLESGGGVVQPGRSLRVSCAASGFTFSNHAMHWVRQAPGKGLEWVAVISYDGSKKFYSDSVRGRFTISRDNSKNTLYLQMNSLRPEDTAVYYCAKGAHGYTSGWHDYWGQGTLVTVSS

SEQ ID NO: 118 - protein, 108 aa, synthetic construct
  source 1..108: note = L3A10[LAG-3] Variable Light
DVVMTQSPSSLSASVGDRVSITCRASQNIGRYLNWYQQKPGKAPKLLVSAASSLQGGVPSRFSGSGSGTDFTLTISRLQPEDFATYFCQQTYSSPQCTFGQGTKVDIK

SEQ ID NO: 119 - protein, 121 aa, synthetic construct
  source 1..121: note = L3C5[LAG-3] Variable Heavy
QVQLVQSGSELKKPGASVKVSCKASGYTFTNYYMHWVRQAPGQGLEWMGIINPSGGATNYAQKFQGRVTMTRDTSTSTVYMELSSLRSEDTAVYYCARDSGYDLGYGMDVWGQGTLVTVSS

SEQ ID NO: 120 - protein, 110 aa, synthetic construct
  source 1..110: note = L3C5[LAG-3] Variable Light
QSVLTQPASVSGSPGQSITISCTGTSSDVGGYNYVSWYQQHPGKAPKLMIYDVSNRPSGASNRFSGSKSGNTASLTISGLQAEDEADYYCSSYTNRNTLLFGGGTKLTVL

SEQ ID NO: 121 - protein, 119 aa, synthetic construct; identical to SEQ ID NO: 115
  source 1..119: note = L3E3[LAG-3] Variable Heavy

SEQ ID NO: 122 - protein, 110 aa, synthetic construct
  source 1..110: note = L3E3[LAG-3] Variable Light
QSVLTQPASASGSPGQSITISCTGTSSDVGGYNYVSWYQQHPGKAPKLMIYDVSNRPSGVSNRFSGSKSGNTASLTISGLQAEDEANYYCSSYTSSSTNVFGTGTKVTVL

SEQ ID NO: 123 - protein, 121 aa, synthetic construct; identical to SEQ ID NO: 79
  source 1..121: note = 7G8[LAG3]_H3.30_L1.34 H3.30 Variable heavy (vh) domain

SEQ ID NO: 124 - protein, 5 aa, synthetic construct
  source 1..5: note = 7G8[LAG3]_H3.30_L1.34 vhCDR1
DAWMS

SEQ ID NO: 125 - protein, 19 aa, synthetic construct
  source 1..19: note = 7G8[LAG3]_H3.30_L1.34 vhCDR2
EISTKANNHATYYAESVKG

SEQ ID NO: 126 - protein, 10 aa, synthetic construct
  source 1..10: note = 7G8[LAG3]_H3.30_L1.34 vhCDR3
LATWDWYFDV

SEQ ID NO: 127 - protein, 111 aa, synthetic construct; identical to SEQ ID NO: 80
  source 1..111: note = 7G8[LAG3]_H3.30_L1.34 L1.34 Variable light (vl) domain

SEQ ID NO: 128 - protein, 15 aa, synthetic construct
  source 1..15: note = 7G8[LAG3]_H3.30_L1.34 vlCDR1
RASQSVDYDGDSYMN

SEQ ID NO: 129 - protein, 7 aa, synthetic construct
  source 1..7: note = 7G8[LAG3]_H3.30_L1.34 vlCDR2
AASELES

SEQ ID NO: 130 - protein, 9 aa, synthetic construct
  source 1..9: note = 7G8[LAG3]_H3.30_L1.34 vlCDR3
QQSNEDPFT

SEQ ID NO: 131 - protein, 114 aa, synthetic construct
  source 1..114: note = N1D
DWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 132 - protein, 114 aa, synthetic construct
  source 1..114: note = N4D
NWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 133 - protein, 114 aa, synthetic construct
  source 1..114: note = D8N
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 134 - protein, 114 aa, synthetic construct
  source 1..114: note = D30N
NWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 135 - protein, 114 aa, synthetic construct
  source 1..114: note = D61N
NWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 136 - protein, 114 aa, synthetic construct
  source 1..114: note = E64Q
NWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 137 - protein, 114 aa, synthetic construct
  source 1..114: note = N65D
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 138 - protein, 114 aa, synthetic construct
  source 1..114: note = Q108E
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 139 - protein, 114 aa, synthetic construct
  source 1..114: note = N1D/D61N
DWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 140 - protein, 114 aa, synthetic construct
  source 1..114: note = N1D/E64Q
DWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 141 - protein, 114 aa, synthetic construct
  source 1..114: note = N4D/D61N
NWVDVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 142 - protein, 114 aa, synthetic construct
  source 1..114: note = N4D/E64Q
NWVDVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 143 - protein, 114 aa, synthetic construct
  source 1..114: note = D8N/D61N
NWVNVISNLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 144 - protein, 114 aa, synthetic construct
  source 1..114: note = D8N/E64Q
NWVNVISNLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 145 - protein, 114 aa, synthetic construct
  source 1..114: note = D61N/E64Q
NWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVQMFINTS

SEQ ID NO: 146 - protein, 114 aa, synthetic construct
  source 1..114: note = E64Q/Q108E
NWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVEMFINTS

SEQ ID NO: 147 - protein, 114 aa, synthetic construct
  source 1..114: note = N1D/N4D/D8N
DWVDVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 148 - protein, 114 aa, synthetic construct
  source 1..114: note = D61N/E64Q/N65D
NWVNVISDLKKIEDLIQSMHIDATLYTESDVHPSCKVTAMKCFLLELQVISLESGDASIHNTVQDLIILANNSLSSNGNVTESGCKECEELEEKNIKEFLQSFVHIVQMFINTS

SEQ ID NO: 149 - protein, 114 aa, synthetic construct
  source 1..114: note = N1D/D61N/E64Q/Q108E
DWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 150 - protein, 114 aa, synthetic construct
  source 1..114: note = N4D/D61N/E64Q/Q108E
NWVDVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 151 - protein, 114 aa, synthetic construct
  source 1..114: note = N1D/N65D
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 152 - protein, 114 aa, synthetic construct
  source 1..114: note = N1D/Q108E
DWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 153 - protein, 114 aa, synthetic construct
  source 1..114: note = N4D/N65D
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 154 - protein, 114 aa, synthetic construct
  source 1..114: note = D30N/N65D
NWVNVISDLKKIEDLIQSMHIDATLYTESNVHPSCKVTAMKCFLLELQVISLESGDASIHDTVEDLIILANNSLSSNGNVTESGCKECEELEEKNIKEFLQSFVHIVQMFINTS

SEQ ID NO: 155 - protein, 114 aa, synthetic construct
  source 1..114: note = D30N/Q108E
NWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVEMFINTS

SEQ ID NO: 156 - protein, 114 aa, synthetic construct
  source 1..114: note = N65D/Q108E
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVEMFINTS

SEQ ID NO: 157 - protein, 114 aa, synthetic construct
  source 1..114: note = E64Q/N65D
NWVNVISDLKKIEDLIQSMHIDATLYTESDVHPSCKVTAMKCFLLELQVISLESGDASIHDTVQDLIILANNSLSSNGNVTESGCKECEELEEKNIKEFLQSFVHIVQMFINTS

SEQ ID NO: 158 - protein, 114 aa, synthetic construct
  source 1..114: note = N1D/N4D/N65D
DWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 159 - protein, 114 aa, synthetic construct
  source 1..114: note = D30N/E64Q/N65D
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 160 - protein, 114 aa, synthetic construct
  source 1..114: note = N4D/D61N/N65D
NWVDVISDLKKIEDLIQSMHIDATLYTESDVHPSCKVTAMKCFLLELQVISLESGDASIHNTVEDLIILANNSLSSNGNVTESGCKECEELEEKNIKEFLQSFVHIVQMFINTS

SEQ ID NO: 161 - protein, 114 aa, synthetic construct
  source 1..114: note = D30N/E64Q/N65D/N71Q/N79Q/N112Q
NWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTS

SEQ ID NO: 162 - protein, 114 aa, synthetic construct
  source 1..114: note = N1D/N4D/D30N/E64Q/N65D/N71Q/N79Q/N112Q
DWVDVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTS

SEQ ID NO: 163 - protein, 114 aa, synthetic construct
  source 1..114: note = N1D/N4D/D30N/E64Q/N65D
NWVDVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 164 - protein, 114 aa, synthetic construct; identical to SEQ ID NO: 163
  source 1..114: note = N4D/D30N/E64Q/N65D

SEQ ID NO: 165 - protein, 114 aa, synthetic construct
  source 1..114: note = D30N/D61N/E64Q/N65D/N71Q/N79Q/N112Q
NWVNVISDLKKIEDLIQSMHIDATLYTESNVHPSCKVTAMKCFLLELQVISLESGDASIHNTVQDLIILAQNSLSSNGQVTESGCKECEELEEKNIKEFLQSFVHIVQMFIQTS

SEQ ID NO: 166 - protein, 114 aa, synthetic construct
  source 1..114: note = D30N/D61N/N65D/N71Q/N79Q/N112Q
NWVNVISDLKKIEDLIQSMHIDATLYTESNVHPSCKVTAMKCFLLELQVISLESGDASIHNTVEDLIILAQNSLSSNGQVTESGCKECEELEEKNIKEFLQSFVHIVQMFIQTS

SEQ ID NO: 167 - protein, 114 aa, synthetic construct
  source 1..114: note = D8N/D30N/E64Q/N65D/N71Q/N79Q/N112Q
NWVNVISNLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTS

SEQ ID NO: 168 - protein, 450 aa, synthetic construct
  source 1..450: note = XENP40735 Chain 1-7G8[LAG3]_H3.30_IgG1_PVA_/S267K/S364K/E357Q/M428L/N434S
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 169 - protein, 435 aa, synthetic construct
  source 1..435: note = XENP40735 Chain 2-human_IL15Ra(sushi)_(GGGGA)5-huma n_IL15(D30N/E64Q/N65D/N71Q/N79Q/N112Q
ITCPPPMSVEHADIWVKSYSLYSRERYICNSGFKRKAGTSSLTECVLNKATNVAHWTTPSLKCIRGGGGAGGGGAGGGGAGGGGAGGGGANWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 170 - protein, 218 aa, synthetic construct
  source 1..218: note = XENP40735 Chain 3-7G8[LAG3]_L1.34_Light Chain
DIVLTQSPSSLSASVGDRVTITCRASQSVDYDGDSYMNWYQQKPGKPPKLLIYAASELESGIPARFSGSGSGTDFTLTISSLQPEDFATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 450 aa, synthetic construct; identical to SEQ ID NO: 168
  source 1..450: note = XENP40736 Chain 1-7G8[LAG3]_H3.30_IgG1_PVA_/S267K/S364K/E357Q/M428L/N434S

SEQ ID NO: 172 - protein, 435 aa, synthetic construct
  source 1..435: note = XENP40736 Chain 2-human_IL15Ra(sushi)_(GGGGS)5-human_IL15(D30N/E64Q/N65D
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 173 - protein, 218 aa, synthetic construct; identical to SEQ ID NO: 170
  source 1..218: note = XENP40736 Chain 3-7G8[LAG3]_L1.34_Light Chain

SEQ ID NO: 174 - protein, 450 aa, synthetic construct; identical to SEQ ID NO: 168
  source 1..450: note = XENP40741 Chain 1-7G8[LAG3]_H3.30_IgG1_PVA_/S267K/S364K/E357Q/M428L/N434S

SEQ ID NO: 175 - protein, 435 aa, synthetic construct
  source 1..435: note = XENP40741 Chain 2-human_IL15Ra(sushi)_(GGGGA)5-huma n_IL15(N1D/N4D/D30N/E64Q/N65D/N71Q/N79Q/N112Q
ITCPPPMSVEHADIWVKSYSLYSRERYICNSGFKRKAGTSSLTECVLNKATNVAHWTTPSLKCIRGGGGAGGGGAGGGGAGGGGAGGGGADWVDVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 176 - protein, 218 aa, synthetic construct; identical to SEQ ID NO: 170
  source 1..218: note = XENP40741 Chain 3-7G8[LAG3]_L1.34_Light Chain

SEQ ID NO: 177 - protein, 450 aa, synthetic construct; identical to SEQ ID NO: 168
  source 1..450: note = XENP40812 Chain 1-7G8[LAG3]_H3.30_IgG1_PVA_/S267K/S364K/E357Q/M428L/N434S

SEQ ID NO: 178 - protein, 435 aa, synthetic construct
  source 1..435: note = XENP40812 Chain 2-human_IL15Ra(sushi)_(GGGGS)5-human_IL15(N1D/N4D/N65D
ITCPPPMSVEHADIWVKSYSLYSRERYICNSGFKRKAGTSSLTECVLNKATNVAHWTTPSLKCIRGGGGSGGGGSGGGGSGGGGSGGGGSDWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 179 - protein, 218 aa, synthetic construct; identical to SEQ ID NO: 170
  source 1..218: note = XENP40812 Chain 3-7G8[LAG3]_L1.34_Light Chain

SEQ ID NO: 180 - protein, 663 aa, synthetic construct
  source 1..663: note = XENP40738 Chain 1-7G8[LAG3]_H3.30_IgG1_pI(-)_Isoste ric_A_C220S/PVA_/S267K/L368D/K370S/M428L/N434S_(GGGGS)2_IL 15Ra(sushi)_(GGGGS)5-IL15(D30N/E64Q/N65D)
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 181 - protein, 450 aa, synthetic construct; identical to SEQ ID NO: 168
  source 1..450: note = XENP40738 Chain 2-7G8[LAG3]_H3.30_IgG1_PVA_/S267K/S364K/E357Q/M428L/N434S

SEQ ID NO: 182 - protein, 218 aa, synthetic construct; identical to SEQ ID NO: 170
  source 1..218: note = XENP40738 Chain 3-7G8[LAG3]_L1.34_Light Chain

SEQ ID NO: 183 - protein, 450 aa, synthetic construct; identical to SEQ ID NO: 168
  source 1..450: note = XENP40814 Chain 1-7G8[LAG3]_H3.30_IgG1_PVA_/S267K/S364K/E357Q/M428L/N434S

SEQ ID NO: 184 - protein, 663 aa, synthetic construct
  source 1..663: note = XENP40814 Chain 2-7G8[LAG3]_H3.30_IgG1_pI(-)_Isoste ric_A_C220S/PVA_/S267K/L368D/K370S/M428L/N434S_(GGGGS)2_IL 15Ra(sushi)_(GGGGS)5-IL15(N4D/D30N/E64Q/N65D)
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 185 - protein, 218 aa, synthetic construct; identical to SEQ ID NO: 170
  source 1..218: note = XENP40814 Chain 3-7G8[LAG3]_L1.34_Light Chain

SEQ ID NO: 186 - protein, 524 aa, synthetic construct
  source 1..524: note = XENP40816 Chain 1-7G8[LAG3]_H3.30_IgG1_PVA_/S267K/S 364K/E357Q/M428L/N434S_(GGGGS)2_IL15Ra(sushi)
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIR

SEQ ID NO: 187 - protein, 573 aa, synthetic construct
  source 1..573: note = XENP40816 Chain 2-7G8[LAG3]_H3.30_IgG1_pI(-)_Isoste ric_A_C220S/PVA_/S267K/L368D/K370S/M428L/N434S_(GGGGS)2-IL 15(N4D/D30N/E64Q/N65D)
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSNWVDVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 188 - protein, 218 aa, synthetic construct; identical to SEQ ID NO: 170
  source 1..218: note = XENP40816 Chain 3-7G8[LAG3]_L1.34_Light Chain

SEQ ID NO: 189 - protein, 662 aa, synthetic construct
  source 1..662: note = XENP40745 Chain 1-Numax[RSV]_H1_IgG1_pI(-)_Isosteri c_A_C220S/PVA_/S267K/L368D/K370S/M428L/N434S_(GGGGS)2_IL15 Ra(sushi)_(GGGGS)5-IL15(D30N/E64Q/N65D/N71Q/N79Q/N112Q)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTS

SEQ ID NO: 190 - protein, 449 aa, synthetic construct
  source 1..449: note = XENP40745 Chain 2-Numax[RSV]_H1-IgG1_PVA_/S267K/S364K/E357Q/M428L/N434S
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 191 - protein, 213 aa, synthetic construct
  source 1..213: note = XENP40745 Chain 3-Numax[RSV]_L1_Light Chain
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 662 aa, synthetic construct
  source 1..662: note = XENP40746 Chain 1-Numax[RSV]_H1_IgG1_pI(-)_Isosteri c_A_C220S/PVA_/S267K/L368D/K370S/M428L/N434S_(GGGGS)2_IL15 Ra(sushi)_(GGGGS)5-IL15(D30N/E64Q/N65D)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 193 - protein, 449 aa, synthetic construct; identical to SEQ ID NO: 190
  source 1..449: note = XENP40746 Chain 2-Numax[RSV]_H1_IgG1_PVA_/S267K/S364K/E357Q/M428L/N434S

SEQ ID NO: 194 - protein, 213 aa, synthetic construct; identical to SEQ ID NO: 191
  source 1..213: note = XENP40746 Chain 3-Numax[RSV]_L1_Light Chain

SEQ ID NO: 195 - protein, 668 aa, synthetic construct
  source 1..668: note = XENP40748 Chain 1-Numax[RSV]_H1_CH1_IL15Ra(sushi)_( GGGGS)5-IL15(D30N/E64Q/N65D)-Fc(216)_IgG1_pI(-)_Isosteric_ A_C220S/PVA_/S267K/L368D/K370S/M428L/N434S
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 196 - protein, 449 aa, synthetic construct; identical to SEQ ID NO: 190
  source 1..449: note = XENP40748 Chain 2-Numax[RSV]_H1_IgG1_PVA_/S267K/S364K/E357Q/M428L/N434S

SEQ ID NO: 197 - protein, 213 aa, synthetic construct; identical to SEQ ID NO: 191
  source 1..213: note = XENP40748 Chain 3-Numax[RSV]_L1_Light Chain

SEQ ID NO: 198 - protein, 449 aa, synthetic construct; identical to SEQ ID NO: 190
  source 1..449: note = XENP40749 Chain 1-Numax[RSV]_H1_IgG1_PVA_/S267K/S364K/E357Q/M428L/N434S

SEQ ID NO: 199 - protein, 435 aa, synthetic construct; identical to SEQ ID NO: 175
  source 1..435: note = XENP40749 Chain 2-human_IL15Ra(sushi)_(GGGGA)5-huma n_IL15(N1D/N4D/D30N/E64Q/N65D/N71Q/N79Q/N112Q

SEQ ID NO: 200 - protein, 213 aa, synthetic construct; identical to SEQ ID NO: 191
  source 1..213: note = XENP40749 Chain 3-Numax[RSV]_L1_Light Chain

SEQ ID NO: 201 - protein, 449 aa, synthetic construct; identical to SEQ ID NO: 190
  source 1..449: note = XENP40750 Chain 1-Numax[RSV]_H1_IgG1_PVA_/S267K/S364K/E357Q/M428L/N434S

SEQ ID NO: 202 - protein, 435 aa, synthetic construct
  source 1..435: note = XENP40750 Chain 2-human_IL15Ra(sushi)_(GGGGA)5-huma n_IL15(D8N/D30N/E64Q/N65D/N71Q/N79Q/N112Q
ITCPPPMSVEHADIWVKSYSLYSRERYICNSGFKRKAGTSSLTECVLNKATNVAHWTTPSLKCIRGGGGAGGGGAGGGGAGGGGAGGGGANWVNVISNLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 203 - protein, 213 aa, synthetic construct; identical to SEQ ID NO: 191
  source 1..213: note = XENP40750 Chain 3-Numax[RSV]_L1_Light Chain

SEQ ID NO: 204 - protein, 449 aa, synthetic construct; identical to SEQ ID NO: 190
  source 1..449: note = XENP40751 Chain 1-Numax[RSV]_H1IgG1_PVA_/S267K/S364K/E357Q/M428L/N434S

SEQ ID NO: 205 - protein, 435 aa, synthetic construct
  source 1..435: note = XENP40751 Chain 2-human_IL15Ra(sushi)_(GGGGA)5-huma n_IL15(D30N/D61N/E64Q/N65D/N71Q/N79Q/N112Q
ITCPPPMSVEHADIWVKSYSLYSRERYICNSGFKRKAGTSSLTECVLNKATNVAHWTTPSLKCIRGGGGAGGGGAGGGGAGGGGAGGGGANWVNVISDLKKIEDLIQSMHIDATLYTESNVHPSCKVTAMKCFLLELQVISLESGDASIHNTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 206 - protein, 213 aa, synthetic construct; identical to SEQ ID NO: 191
  source 1..213: note = XENP40751 Chain 3-Numax[RSV]_L1_Light Chain

SEQ ID NO: 207 - protein, 449 aa, synthetic construct; identical to SEQ ID NO: 190
  source 1..449: note = XENP40752 Chain 1-Numax[RSV]_H1_IgG1_PVA_/S267K/S364K/E357Q/M428L/N434S

SEQ ID NO: 208 - protein, 435 aa, synthetic construct
  source 1..435: note = XENP40752 Chain 2-human_IL15Ra(sushi)_(GGGGA)5-huma n_IL15(D30N/D61N/N65D/N71Q/N79Q/N112Q
ITCPPPMSVEHADIWVKSYSLYSRERYICNSGFKRKAGTSSLTECVLNKATNVAHWTTPSLKCIRGGGGAGGGGAGGGGAGGGGAGGGGANWVNVISDLKKIEDLIQSMHIDATLYTESNVHPSCKVTAMKCFLLELQVISLESGDASIHNTVEDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 209 - protein, 213 aa, synthetic construct; identical to SEQ ID NO: 191
  source 1..213: note = XENP40752 Chain 3-Numax[RSV]_L1_Light Chain

SEQ ID NO: 210 - protein, 449 aa, synthetic construct; identical to SEQ ID NO: 190
  source 1..449: note = XENP40813 Chain 1-Numax[RSV]_H1_IgG1_PVA_/S267K/S364K/E357Q/M428L/N434S

SEQ ID NO: 211 - protein, 435 aa, synthetic construct; identical to SEQ ID NO: 178
  source 1..435: note = XENP40813 Chain 2-human_IL15Ra(sushi)_(GGGGA)5-human_IL15(N1D/N4D/N65D

SEQ ID NO: 212 - protein, 213 aa, synthetic construct; identical to SEQ ID NO: 191
  source 1..213: note = XENP40813 Chain 3-Numax[RSV]_L1_Light Chain

SEQ ID NO: 213 - protein, 449 aa, synthetic construct; identical to SEQ ID NO: 190
  source 1..449: note = XENP40815 Chain 1-Numax[RSV]_H1_IgG1_PVA_/S267K/S364K/E357Q/M428L/N434S

SEQ ID NO: 214 - protein, 662 aa, synthetic construct
  source 1..662: note = XENP40815 Chain 2-Numax[RSV]_H1_IgG1_pI(-)_Isosteri c_A_C220S/PVA_/S267K/L368D/K370S/M428L/N434S_(GGGGS)2_IL15 Ra(sushi)_(GGGGS)5-IL15(N4D/D30N/E64Q/N65D)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 215 - protein, 213 aa, synthetic construct; identical to SEQ ID NO: 191
  source 1..213: note = XENP40815 Chain 3-Numax[RSV]_L1_Light Chain

SEQ ID NO: 216 - protein, 663 aa, synthetic construct
  source 1..663: note = XENP40737 Chain 1-7G8[LAG3]_H3.30_IgG1_pI(-)_Isoste ric_A_C220S/PVA_/S267K/L368D/K370S/M428L/N434S_(GGGGS)2_IL 15Ra(sushi)_(GGGGS)5-IL15(D30N/E64Q/N65D/N71Q/N79Q/N112Q)
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTS

SEQ ID NO: 217 - protein, 450 aa, synthetic construct; identical to SEQ ID NO: 168
  source 1..450: note = XENP40737 Chain 2-7G8[LAG3]_H3.30_IgG1_PVA_/S267K/S364K/E357Q/M428L/N434S

SEQ ID NO: 218 - protein, 218 aa, synthetic construct; identical to SEQ ID NO: 170
  source 1..218: note = XENP40737 Chain 3-7G8[LAG3]_L1.34_Light Chain

SEQ ID NO: 219 - protein, 669 aa, synthetic construct
  source 1..669: note = XENP40739 Chain 1-7G8[LAG3]_H3.30_CH1_IL15Ra(sushi) _(GGGGS)5-IL15(D30N/E64Q/N65D/N71Q/N79Q/N112Q)-Fc(216)_IgG 1_pI(-)_Isosteric_A_C220S/PVA_/S267K/L368D/K370S/M428L/N43 4S
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 220 - protein, 450 aa, synthetic construct; identical to SEQ ID NO: 168
  source 1..450: note = XENP40739 Chain 2-7G8[LAG3]_H3.30_IgG1_PVA_/S267K/S364K/E357Q/M428L/N434S

SEQ ID NO: 221 - protein, 218 aa, synthetic construct; identical to SEQ ID NO: 170
  source 1..218: note = XENP40739 Chain 3-7G8[LAG3]_L1.34_Light Chain

SEQ ID NO: 222 - protein, 669 aa, synthetic construct
  source 1..669: note = XENP40740 Chain 1-7G8[LAG3]_H3.30_CH1_IL15Ra(sushi) _(GGGGS)5-IL15(D30N/E64Q/N65D)-Fc(216)_IgG1_pI(-)_Isosteri c_A_C220S/PVA_/S267K/L368D/K370S/M428L/N434S
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 223 - protein, 450 aa, synthetic construct; identical to SEQ ID NO: 168
  source 1..450: note = XENP40740 Chain 2-7G8[LAG3]_H3.30-IgG1_PVA_/S267K/S364K/E357Q/M428L/N434S

SEQ ID NO: 224 - protein, 218 aa, synthetic construct; identical to SEQ ID NO: 170
  source 1..218: note = XENP40740 Chain 3-7G8[LAG3]_L1.34_Light ChainXYX

SEQ ID NO: 225 - protein, 450 aa, synthetic construct; identical to SEQ ID NO: 168
  source 1..450: note = XENP40742 Chain 1-7G8[LAG3]_H3.30_IgG1_PVA_/S267K/S364K/E357Q/M428L/N434S

SEQ ID NO: 226 - protein, 435 aa, synthetic construct; identical to SEQ ID NO: 202
  source 1..435: note = XENP40742 Chain 2-human_IL15Ra(sushi)_(GGGGA)5-huma n_IL15(D8N/D30N/E64Q/N65D/N71Q/N79Q/N112Q

SEQ ID NO: 227 - protein, 218 aa, synthetic construct; identical to SEQ ID NO: 170
  source 1..218: note = XENP40742 Chain 3-7G8[LAG3]_L1.34_Light Chain

SEQ ID NO: 228 - protein, 450 aa, synthetic construct; identical to SEQ ID NO: 168
  source 1..450: note = XENP40743 Chain 1-7G8[LAG3]_H3.30_IgG1_PVA_/S267K/S364K/E357Q/M428L/N434S

SEQ ID NO: 229 - protein, 435 aa, synthetic construct; identical to SEQ ID NO: 205
  source 1..435: note = XENP40743 Chain 2-human_IL15Ra(sushi)_(GGGGA)5-huma n_IL15(D30N/D61N/E64Q/N65D/N71Q/N79Q/N112Q

SEQ ID NO: 230 - protein, 218 aa, synthetic construct; identical to SEQ ID NO: 170
  source 1..218: note = XENP40743 Chain 3-7G8[LAG3]_L1.34_Light Chain

SEQ ID NO: 231 - protein, 450 aa, synthetic construct; identical to SEQ ID NO: 168
  source 1..450: note = XENP40744 Chain 1-7G8[LAG3]_H3.30_IgG1_PVA_/S267K/S364K/E357Q/M428L/N434S

SEQ ID NO: 232 - protein, 435 aa, synthetic construct; identical to SEQ ID NO: 208
  source 1..435: note = XENP40744 Chain 2-human_IL15Ra(sushi)_(GGGGA)5-huma n_IL15(D30N/D61N/N65D/N71Q/N79Q/N112Q

SEQ ID NO: 233 - protein, 218 aa, synthetic construct; identical to SEQ ID NO: 170
  source 1..218: note = XENP40744 Chain 3-7G8[LAG3]_L1.34_Light Chain

SEQ ID NO: 234 - protein, 442 aa, synthetic construct
  source 1..442: note = XENP16432 Chain 1- Nivolumab_H0L0_IgG1_PVA/S267K Heavy Chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 214 aa, synthetic construct
  source 1..214: note = XENP16432 Chain 2- Nivolumab_L0_Light Chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 450 aa, synthetic construct
  source 1..450: note = XENP30473 Chain 1- 7G8[LAG3]_H3.30_IgG1_PVA_/S267K_Heavy Chain
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 218 aa, synthetic construct; identical to SEQ ID NO: 170
  source 1..218: note = XENP30473 Chain 2- 7G8[LAG3]_L1.34_Light Chain

SEQ ID NO: 238 - protein, 523 aa, synthetic construct
  source 1..523: note = XENP40817 Chain 1-Numax[RSV]_H1_IgG1_PVA_/S267K/S36 4K/E357Q/M428L/N434S-(GGGGS)2_IL15Ra(sushi)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGGGGGSGGGGSITCPPPMSVEHADIWVKSYSLYSRERYICNSGFKRKAGTSSLTECVLNKATNVAHWTTPSLKCIR

SEQ ID NO: 239 - protein, 572 aa, synthetic construct
  source 1..572: note = XENP40817 Chain 2-Numax[RSV]_H1_IgG1_pI(-)_Isosteri c_A_C220S/PVA_/S267K/L368D/K370S/M428L/N434S_(GGGGS)2_IL15 (N4D/D30N/E64Q/N65D)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGGGGGSGGGGSNWVDVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 240 - protein, 213 aa, synthetic construct; identical to SEQ ID NO: 191
  source 1..213: note = XENP40817 Chain 3-Numax[RSV]_L1_Light Chain

SEQ ID NO: 241 - protein, 668 aa, synthetic construct
  source 1..668: note = XENP40747 Chain 1-Numax[RSV]_H1_CH1_IL15Ra(sushi)_( GGGGS)5-IL15(D30N/E64Q/N65D/N71Q/N79Q/N112Q)-Fc(216)_IgG1_ pI(-)_Isosteric_A_C220S/PVA_/S267K/L368D/K370S/M428L/N434S
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILAQNSLSSNGQVTESGCKECEELEEKNIKEFLQSFVHIVQMFIQTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 242 - protein, 449 aa, synthetic construct; identical to SEQ ID NO: 190
  source 1..449: note = XENP40747 Chain 2-Numax[RSV]_H1_IgG1_PVA_/S267K/S364K/E357Q/M428L/N434S

SEQ ID NO: 243 - protein, 213 aa, synthetic construct; identical to SEQ ID NO: 191
  source 1..213: note = XENP40747 Chain 3-Numax[RSV]_L1_Light Chain

SEQ ID NO: 244 - protein, 5 aa, synthetic construct
GGGGS

SEQ ID NO: 245 - protein, 5 aa, synthetic construct
GGGGA